(19) United States
(12) Patent Application Publication
(10) Pub. No.: US 2003/0232787 A1
(43) Pub. Date: Dec. 18, 2003

(54) Combinations of an endothelin receptor antagonist and an antiepileptic compound having pain alleviating properties or analgesic

(76) Inventor: David James Dooley, South Lyon, MI (US)

Correspondence Address: David R Kurlandsky, Warner-Lambert Company, 2800 Plymouth Road, Ann Arbor, MI, 48105, US

(21) Appl. No.: 10/296,792

(22) PCT Filed: May 8, 2001
(86) PCT No.: PCT/US01/14793

(51) Int. Cl. A61K 31/69; A61K 31/675; A61K 31/542; A61K 31/415; A61K 31/485; A61K 31/19; A61K 31/195

(52) U.S. Cl. 514/64; 514/561; 514/226.5; 514/381; 514/80; 514/419; 514/282; 514/161; 514/406; 514/570

(57) ABSTRACT

The present invention is a novel combination effective for alleviating pain comprising a pain alleviating effective amount of an endothelin receptor antagonist or a pharmaceutically acceptable salt thereof and from 1 to 3 compounds independently selected from the group consisting of antiepileptic compounds having pain alleviating properties and analgesics, and pharmaceutically acceptable salts thereof, and pharmaceutical compositions comprising same. The administration of endothelin receptor antagonists in these novel combinations results in an improved reduction in the frequency and severity of pain. The incidence of unwanted side effects can be reduced by these novel combinations in comparison to using higher doses of a single agent treatment to achieve a similar therapeutic effect. The present invention is also directed to methods of using effective amounts of the novel combinations and pharmaceutical compositions thereof to treat pain in mammals, including a human.

The present invention is directed to novel combinations effective for alleviating pain comprising a pain alleviating effective amount of an endothelin receptor antagonist or a pharmaceutically acceptable salt thereof, and one or two compounds selected from the group consisting of antiepileptic compounds having pain alleviating properties and analgesics, and to methods of treating or alleviating pain comprising administering effective amounts of said combinations to a mammal, including a human, in need of said treatment. The invention also is directed to pharmaceutical compositions comprising said combinations.

### BACKGROUND OF THE INVENTION

A number of treatments involving the administration of single drugs are currently recommended for pain relief. It is well-known that single administration of opioid and nonopioid analgesics have been shown to display pain alleviating properties. Some antiepileptic compounds, such as gabapentin and pregabalin, have also demonstrated pain alleviating properties.

Neurokinin 1 (NK₁) receptor antagonists are being developed for the treatment of a number of physiological disorders associated with an excess or imbalance of tachykinins, and in particular of substance P, an important neurotransmitter in the transmission of pain. The selective NK₁ receptor antagonist, [2-(1H-indol-3-yl)-1-methyl-1-(1-phenyl-ethylcarbamoyl)-ethyl]-carbamic acid benzofuran-2-ylmethyl ester, has been shown to block the maintenance of streptozotocin-induced static allodynia in the rat (Field et al., J. Pharmacol. Exp. Ther. 1998;285:1226-1232).

Recently, the endothelin receptor antagonist ABT-627 was found to attenuate pain in a rat model of diabetic neuropathy (Jarvis M.F. et al., Eur. J. Pharmacol. 2000;388:29-35). The antihyperalgesic effects are maintained after chronic administration. This effect probably results from a block of ET_{A} receptors activated by endogenous endothelin-1, leading to deficits in neurovascular blood flow and nerve conduction velocities. Recently, another endothelin receptor antagonist, BQ-123, was shown to alleviate pain in a mouse model of inflammation and hyperalgesia (Piovezan A. P. et al., Brit. J. Pharmacol. 2000;129:961-968).

More recently, studies have shown that both endothelin A receptors and endothelin B receptors are involved in signaling pain, namely acute or neuropathic pain and chronic inflammatory pain, respectively (Pomonis J. D. et al., J. Neurosci. 2001;21(3):999-1006).

Endothelin-1 (ET-1), a potent vasoconstrictor, is a 21 amino acid bicyclic peptide that was first isolated from cultured porcine aortic endothelial cells. Endothelin-1 is one of a family of structurally similar bicyclic peptides which include ET-2, ET-3, vasoactive intestinal contractor (VIC), and the sarafotoxins (SRTXs).

Specific endothelin receptor antagonists that can be used herein are disclosed in:

U.S. Pat. No. 5,599,811 is incorporated by reference.

U.S. Pat. No. 5,482,960 is incorporated by reference.

Further, the following U.S. patents recite certain endothelin antagonists and are incorporated by reference herein.

U.S. Pat. Nos.:

5,382,569;

5,773,414;

5,641,752;

5,260,276;

5,922,681;

5,691,373;

6,017,916;

5,610,177;

5,658,943;

5,922,759;

6,051,599;

6,043,265;

6,043,241;

6,040,309;

6,017,951;

5,998,468;

6,030,991;

6,030,970;

6,022,886;

6,020,348;

6,017,952;

6,017,945;

6,017,939;

6,013,655;

6,008,224;

6,004,965;

5,986,103;

5,985,886;

5,977,117;

5,977,075;

5,969,151;

5,968,971;

5,965,732;

5,962,682;

5,962,490;

5,958,968;

5,958,905;

5,948,754;

5,942,516;

5,939,446;

5,929,116;

5,929,106;

5,925,731;

5,916,907;

5,888,972;

5,883,092;

5,883,090;

5,883,075;

5,866,568;

5,861,401;

5,856,509;

5,856,484;

5,846,990;

5,846,985;

5,840,722;

5,834,483;

5,834,469;

5,827,869;

5,821,256;

5,817,693;

5,817,683;

5,817,653;

5,804,585;

5,780,498;

5,780,473;

5,767,144;

5,760,038;

5,739,333;

5,736,564;

5,731,434;

5,731,321;

5,728,706;

5,726,194;

5,719,183;

5,719,182;

5,716,985;

5,716,984;

5,714,479;

5,703,106;

5,700,807;

5,693,637;

5,691,315;

5,686,481;

5,686,478;

5,668,176;

5,668,137;

5,658,902;

5,656,604;

5,654,309;

5,641,793;

5,639,860;

5,631,222;

5,622,971;

5,616,684;

5,614,498;

5,614,497;

5,612,359;

5,594,021;

5,591,761;

5,589,478;

5,587,505;

5,576,439;

5,571,821;

5,565,485;

5,559,135;

5,559,105;

5,550,138;

5,550,110;

5,538,991;

5,538,950;

5,514,696;

5,514,691;

5,496,928;

5,494,897;

5,492,917;

5,492,892;

5,470,833;

5,464,853;

5,463,107;

5,444,152;

5,430,022;

5,420,133;

5,420,123;

5,401,745;

5,391,566;

5,389,620;

5,380,921;

5,378,715;

5,374,638;

5,352,659;

5,334,598;

5,248,807;

5,240,910;

5,187,195; and

5,114,918.

Further, the following PCT patent applications recite certain endothelin antagonists and are incorporated herein by reference.

WO Publication Number:

00/09489;

00/01389;

99/63936;

99/56761;

99/49866;

99/48530;

99/45002;

99/44988;

99/42453;

99/37639;

99/36408;

99/29685;

99/27934;

99/25701;

99/23078;

99/20623;

99/12916;

99/11629;

99/06397;

99/05132;

99/02519;

98/58916;

98/57938;

98/57933;

98/49162;

98/42709;

98/42702;

98/41521;

98/41515;

98/33780;

98/27091;

98/27077;

98/27070;

98/09953;

98/08836;

97/37987;

97/37986;

97/37985;

97/30046;

97/30045;

97/28158;

97/28154;

97/25321;

97/19077;

97/17340;

97/13758;

96/04905;

95/26360;

94/21255;

94/02474;

93/23404; and

93/08799.

Specific antiepileptic compounds having pain alleviating properties are incorporated by reference and are disclosed in:

U.S. Pat. No. 4,024,175;

U.S. Pat. No. 4,087,544;

U.S. Pat. No. 4,960,931;

U.S. Pat. No. 5,563,175; and

WO Publications No. WO 92/09560;

WO 93/23383;

WO 98/17627;

WO 99/31057;

WO 99/31074; and

WO 99/31075.

PCT/US 98/23991 is incorporated by reference.

The NK₁ receptor antagonists, such as capsaicin, heretofore used to treat pain or a disorder exhibiting a pain component can be used herein. Specific NK₁ receptor antagonists that can be used herein are disclosed in U.S. Pat. Nos. 3,862,114, 3,912,711, 4,472,305, 4,481,139, 4,680,283, 4,839,465, 5,102,667, 5,162,339, 5,164,372, 5,166,136, 5,232,929, 5,242,944, 5,300,648, 5,310,743, 5,338,845, 5,340,822, 5,378,803, 5,410,019, 5,411,971, 5,420,297, 5,422,354, 5,446,052, 5,451,586, 5,525,712, 5,527,811, 5,536,737, 5,541,195, 5,594,022, 5,561,113, 5,576,317, 5,604,247, 5,624,950, and 5,635,510; World Patent Application Nos. WO 90/05525, WO 91/09844, WO 91/12266, WO 92/06079, WO 92/12151, WO 92/15585, WO 92/20661, WO 92/20676, WO 92/21677, WO 92/22569, WO 93/00330, WO 93/00331, WO 93/01159, WO 93/01160, WO 93/01165, WO 93/01169, WO 93/01170, WO 93/06099, WO 93/10073, WO 93/14084, WO 93/19064, WO 93/21155, WO 94/04496, WO 94/08997, WO 94/29309, WO 95/11895, WO 95/14017, WO 97/19942, WO 97/24356, WO 97/38692, WO 98/02158, and WO 98/07694; European Patent Application Nos. 284942, 327009, 333174, 336230, 360390, 394989, 428434, 429366, 443132, 446706, 484719, 499313, 512901, 512902, 514273, 514275, 515240, 520555, 522808, 528495, 532456, and 591040.

U.S. Pat. No. 5,800,385 and WO 96/19233 are related and recite irrigation solutions comprised of anti-pain/anti-inflammation agents selected from: (1) serotonin receptor antagonists; (2) serotonin receptor agonists; (3) histamine receptor antagonists; (4) bradykinin receptor antagonists; (5) kallikrein inhibitors; (6) tachykinin receptor antagonists, including neurokinin₁ and neurokinin₂ receptor subtype antagonists; (7) calcitonin gene-related peptide (CGRP) receptor antagonists; (8) interleukin receptor antagonists; (9) inhibitors of enzymes active in the synthetic pathway for arachadonic acid metabolites, including (a) phospholipase inhibitors, including PLA₂ isoform inhibitors and PLC_{γ} isoform inhibitors, (b) cyclooxygenase inhibitors, and (c) lipoxygenase inhibitors; (10) prostanoid receptor antagonists including eicosanoid EP-1 and EP-4 receptor subtype antagonists and thromboxane receptor subtype antagonists; (11) leukotriene receptor antagonists including leukotriene B₄ receptor subtype antagonists and leukotriene D₄ receptor subtype antagonists; (12) opioid receptor agonists, including mu-opiate, delta opiate, and kappa opiate receptor subtype agonists; (13) purinoceptor agonists and antagonists including P_{2X} receptor antagonists and P_{2Y} receptor agonists; (14) adensosine triphosphate (ATP)-sensitive potassium channel openers; and (15) calcium channel antagonists. Further recited are irrigation solutions comprised of anti-pain/anti-inflammation agents as described above and an anti-spasm agent selected from anti-pain/anti-inflammation agents which also serve as an anti-spasm agent such as serotonin receptor antagonists, tachykinin receptor antagonists, ATP-sensitive potassium channel openers, and calcium channel antagonists and agents utilized specifically for their anti-spasm properties such as endothelin receptor antagonists and the nitric oxide donors (enzyme activators).

WO 96/35453 recites methods of preventing/treating preterm labor, imminent abortion, dysmenorrhea, menstrual disorders (i.e., dysfunctional uterine bleeding, menorrhagia, breakthrough bleeding), preeclampsia of pregnancy which utilize an endothelin antagonist and/or an endothelin synthase (ECE) inhibitor which may be used in combination with a progestational agent (progestin), a cyclooxygenase inhibitor and/or a nitric oxide (NO) donor and/or NO substrate. Further recited are methods of preventing/treating atherosclerotic vascular disease and hypertension with a combination of an endothelin antagonist and/or endothelin synthase inhibitor with steroid hormones, and/or with an NO donor and/or NO substrate, and optionally with a cyclooxygenase inhibitor.

Despite the benefits derived from current single drug pain relief regimens, these regimens have disadvantages. One area of concern relates to the incidence of unwanted side effects caused by many of the pain treatment regimens available today. Opioid analgesics, such as morphine, are sparingly prescribed for pain because of the well-known addictive effects and significant central nervous system (CNS) side effects and gastrointestinal side effects. Among nonopioid drugs often used alone for treatment of pain, nonsteroidal anti-inflammatory drugs (NSAIDs), such as ibuprofen and naproxen, are criticized for their irritation of the gastrointestinal tract.

Another concern of current pain treatment regimens relates to their effectiveness. Many single active ingredients employed in current pain relief regimens cannot achieve adequate pain alleviation even at their maximum therapeutic approved doses in some severe pain states. In addition to not achieving adequate pain alleviation, increasing the drug dose may produce an increase in unwanted side effects such as cognitive impairment, nausea, and constipation.

In view of these concerns, it is evident that there is a need for an improved pain regimen that provides an improved therapeutic benefit (i.e., reduced severity and/or frequency of pain) and/or reduces the incidence of unwanted side effects caused by many of the current regimens.

A combination of pain alleviating compounds including an endothelin receptor antagonist with one, two, or three known compounds selected from the group consisting of antiepileptic compounds having pain alleviating properties and analgesics would provide benefits to patients in need of pain alleviating treatment not provided by administration of a higher dose of any single compound to achieve similar therapeutic benefit or by a combination of two or more pain alleviating compounds that did not include an endothelin receptor antagonist.

The inventors have now surprisingly found that endothelin receptor antagonists, when co-administered with compounds selected from the group consisting of antiepileptics having pain alleviating properties or analgesics, result in unexpected improved pain relief. This finding is unexpected as the combination of an endothelin antagonist, which modulates diseases with a vascular component, and a pain alleviating agent to treat pain is not a combination that would have been obvious to one skilled in the medical arts at the time the invention was made.

All that is required to practice the present invention is to administer a therapeutically effective amount of the invention combination from 1 to 6 times daily to a patient in need of treatment for alleviation of pain. As discussed below, determination of dosage forms, amounts of the invention combinations, and routes of administration, selection of compounds for inclusion in the invention combination, and identification of patients in need of treatment is within the level of ordinary skill in the medical and pharmaceutical arts.

### SUMMARY OF THE INVENTION

The present invention is combinations for alleviating pain. The combinations comprise a pain alleviating effective amount of an endothelin receptor antagonist or a pharmaceutically acceptable salt thereof and from 1 to 3 compounds independently selected from the group consisting of antiepileptic compounds having pain alleviating properties and analgesics, and pharmaceutically acceptable salts thereof. Nonlimiting examples of antiepileptic compounds having pain alleviating properties are gabapentin and pregabalin. Analgesics are selected from opioid analgesics and nonopioid analgesics. Nonlimiting examples of nonopioid analgesics include NSAIDs, N-methyl-D-aspartate (NMDA) receptor antagonists, and neurokinin 1 (NK₁) receptor antagonists.

In a preferred embodiment of the present invention, antiepileptics having pain alleviating properties include those that have the following Formula I:

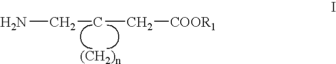

wherein R₁ is hydrogen or a lower alkyl; n is an integer of from 4 to 6; and the cyclic ring is optionally substituted by lower alkyl or lower cycloalkyl, and the pharmaceutically acceptable salts thereof. The term lower alkyl includes straight or branched chain alkyl groups of up to eight carbon atoms. An especially preferred embodiment utilizes a compound of Formula I where R₁ is hydrogen and n is 5, which compound is 1-(aminomethyl)-cyclohexane acetic acid, known generically as gabapentin.

Other preferred compounds of Formula I above include, but are not limited to, ethyl 1-aminomethyl-1-cyclohexane-acetate, 1-aminomethyl-1-cycloheptane-acetic acid, 1-aminomethyl-1-cyclopentane-acetic acid, methyl-1-aminomethyl-1-cyclohexane-acetate, n-butyl 1-aminomethyl-1-cyclohexane-acetate, methyl 1-aminomethyl-1-cycloheptane-acetate, n-butyl 1-aminomethyl-1-cycloheptane-acetate, toluene sulfonate, 1-aminomethyl-1-cyclopentane-acetate, benzene-sulfonate, and n-butyl 1-aminomethyl-1-cyclopentane-acetate.

Other preferred compounds of Formula I above, wherein the cyclic ring is substituted for example with alkyl such as methyl or ethyl, include, but are not limited to (1-aminomethyl-3-methylcyclohexyl)acetic acid, (1-aminomethyl-3-methylcyclopentyl)acetic acid, and (1-aminomethyl-3,4-dimethylcyclopentyl)-acetic acid.

In another preferred embodiment of the present invention, antiepileptics having pain alleviating properties include those that are included in Formula II:

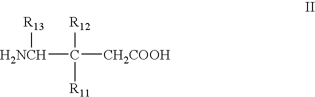

wherein R₁₁ is a straight or branched alkyl of from 1 to 6 carbon atoms, phenyl, or cycloalkyl having from 3 to 6 carbon atoms; R₁₂ is hydrogen or methyl; and R₁₃ is hydrogen, methyl, or carboxyl; or an individual diastereomeric or enantiomeric isomer thereof; or a pharmaceutically acceptable salt thereof.

The most preferred compound of Formula II is where R₁₂ and R₁₃ are both hydrogen, and R₁₁ is —(CH₂)₀₋₂-iC₄H₉ as an (R), (S), or (R,S) isomer. A more preferred embodiment of the invention utilizes 3-aminomethyl-5-methyl-hexanoic acid, and especially (S)-3-(aminomethyl)-5-methylhexanoic acid, now known generically as pregabalin. Pregabalin is also known as “CI-1008” and “S-(+)-IBG.” Another preferred compound is 3-(1-aminoethyl)-5-methylhexanoic acid.

Another preferred embodiment of the present invention includes an antiepileptic compound having pain alleviating properties of Formula

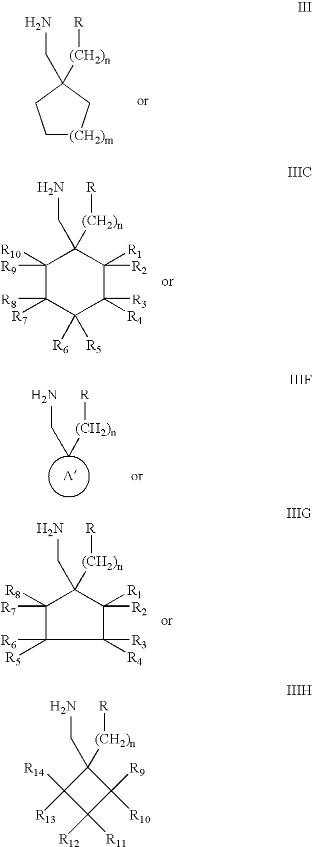

or a pharmaceutically acceptable salt thereof wherein:

n is an integer of from 0 to 2;

m is an integer of from 0 to 3;

R is sulfonamide,

amide,

phosphonic acid,

heterocycle,

sulfonic acid, or

hydroxamic acid;

A′ is a bridged ring selected from

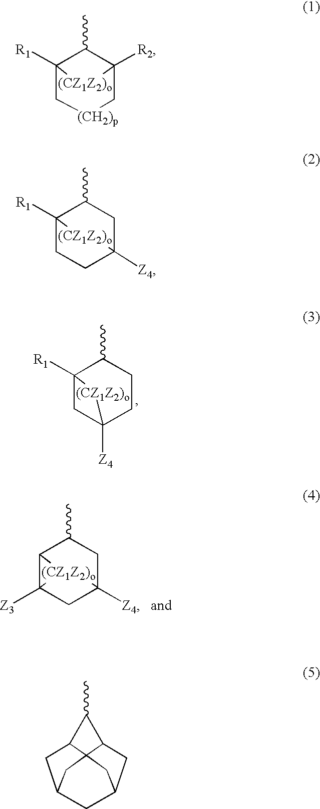

wherein

[symbol] is the point of attachment;

Z₁ to Z₄ are each independently selected from hydrogen and methyl;

o is an integer of from 1 to 4; and

p is an integer of from 0 to 2.

In formula (1) above, R cannot be sulfonic acid when m is 2 and n is 1 (Suman-Chaulan N. et al., European Journal of Pharmacology, 1993;244:293-301).

Another preferred embodiment of the present invention includes a compound of Formula III, IIIC, IIIF, IIIG, or IIIH selected from:

(1-Aminomethyl-cyclohexylmethyl)-phosphonic acid;

(1R-trans)(1-Aminomethyl-3-methyl-cyclohexylmethyl)-phosphonic acid;

(trans)(1-Aminomethyl-3,4-dimethyl-cyclopentylmethyl)-phosphonic acid;

(1R-trans)(1-Aminomethyl-3-methyl-cyclopentylmethyl)-phosphonic acid;

(1S-cis)(1-Aminomethyl-3-methyl-cyclopentylmethyl)-phosphonic acid;

(1S-trans)(1-Aminomethyl-3-methyl-cyclopentylmethyl)-phosphonic acid;

(1R-cis)(1-Aminomethyl-3-methyl-cyclopentylmethyl)-phosphonic acid;

(1α,3α,4α)(1-Aminomethyl-3,4-dimethyl-cyclopentylmethyl)-phosphonic acid;

(1α,3β,4β)(1-Aminomethyl-3,4-dimethyl-cyclopentylmethyl)-phosphonic acid;

(R)(1-Aminomethyl-3,3-dimethyl-cyclopentylmethyl)-phosphonic acid;

(S)(1-Aminomethyl-3,3-dimethyl-cyclopentylmethyl)-phosphonic acid;

(1-Aminomethyl-3,3-dimethyl-cyclobutylmethyl)-phosphonic acid;

2-(1-Aminomethyl-cyclohexyl)-N-hydroxy-acetamide;

(1S-trans)2-(1-Aminomethyl-3-methyl-cyclohexyl)-N-hydroxy-acetamide;

(trans)2-(1-Aminomethyl-3,4-dimethyl-cyclopentyl)-N-hydroxy-acetamide;

(1S-cis)2-(1-Aminomethyl-3-methyl-cyclopentyl)-N-hydroxy-acetamide;

(1R-trans)2-(1-Aminomethyl-3-methyl-cyclopentyl)-N-hydroxy-acetamide;

(1R-cis)2-(1-Aminomethyl-3-methyl-cyclopentyl)-N-hydroxy-acetamide;

(1S-trans)2-(1-Aminomethyl-3-methyl-cyclopentyl)-N-hydroxy-acetamide;

(1α,3α,4α)2-(1-Aminomethyl-3,4-dimethyl-cyclopentyl)-N-hydroxy-acetamide;

(1α,3β,4β)2-(1-Aminomethyl-3,4-dimethyl-cyclopentyl)-N-hydroxy-acetamide;

(S)2-(1-Aminomethyl-3,3-dimethyl-cyclopentyl)-N-hydroxy-acetamide;

(R)2-(1-Aminomethyl-3,3-dimethyl-cyclopentyl)-N-hydroxy-acetaminde;

2-(1-Aminomethyl-3,3-dimethyl-cyclobutyl)-N-hydroxy-acetamide;

N-[2-(1-Aminomethyl-cyclohexyl)-ethyl]-methanesulfonamide;

(1S-cis)N-[2-(1-Aminomethyl-3-methyl-cyclohexyl)-ethyl]-methanesulfonamide;

(trans)N-[2-(1-Aminomethyl-3,4-dimethyl-cyclopentyl)-ethyl]-methanesulfonamide;

(1S-cis)N-[2-(1-Aminomethyl-3-methyl-cyclopentyl)-ethyl]-methanesulfonamide;

(1R-trans)N-[2-(1-Aminomethyl-3-methyl-cyclopentyl)-ethyl]-methanesulfonamide;

(1R-cis)N-[2-(1-Aminomethyl-3-methyl-cyclopentyl)-ethyl]-methanesulfonamide;

(1S-cis)N-[2-(1-Aminomethyl-3-methyl-cyclopentyl)-ethyl]-methanesulfonamide;

(1α,3α,4α)N-[2-(1-Aminomethyl-3,4-dimethyl-cyclopentyl)-ethyl]-methanesulfonamide;

(1α,3β,4β)N-[2-(1-Aminomethyl-3,4-dimethyl-cyclopentyl)-ethyl]-methanesulfonamide;

(S)N-[2-(1-Aminomethyl-3,3-dimethyl-cyclopentyl)-ethyl]-methanesulfonamide;

(R)N-[2-(1-Aminomethyl-3,3-dimethyl-cyclopentyl)-ethyl]-methanesulfonamide;

N-[2-(1-Aminomethyl-3 ,3-dimethyl-cyclobutyl)-ethyl]-methanesulfonamide;

3-(1-Aminomethyl-cyclohexylmethyl)-4H-[1,2,4]oxadiazol-5-one;

3-(1-Aminomethyl-cyclohexylmethyl)-4H-[1,2,4]oxadiazol-5-one hydrochloride;

(1S-cis)3-(1-Aminomethyl-3-methyl-cyclohexylmethyl)-4H-[1,2,4]oxadiazol-5-one;

(trans)3-(1-Aminomethyl-3,4-dimethyl-cyclopentylmethyl)-4H-[1,2,4]oxadiazol-5-one;

(1S-cis)3-(1-Aminomethyl-3-methyl-cyclopentyhnethyl)-4H-[1,2,4]oxadiazol-5-one;

(1R-trans)3-(1-Aminomethyl-3-methyl-cyclopentylmethyl)-4H-[1,2,4]oxadiazol-5-one;

(1R-cis)3-(1-Aminomethyl-3-methyl-cyclopentylmethyl)-4H-[1,2,4]oxadiazol-5-one;

(1S-trans)3-(1-Aminomethyl-3-methyl-cyclopentylmethyl)-4H-[1,2,4]oxadiazol-5-one;

(1α,3α,4α)3-(1-Aminomethyl-3,4-dimethyl-cyclopentylmethyl)-4H-[1,2,4]oxadiazol-5-one;

(1α,3β,4β)3-(1-Aminomethyl-3,4-dimethyl-cyclopentylmethyl)-4H-[1,2,4]oxadiazol-5-one;

(S)3-(1-Aminomethyl-3,3-dimethyl-cyclopentylmethyl)-4H-[1,2,4]oxadiazol-5-one;

(R)3-(1-Aminomethyl-3,3-dimethyl-cyclopentylmethyl)-4H-[1,2,4]oxadiazol-5-one;

3-(1-Aminomethyl-3,3-dimethyl-cyclobutylmethyl)-4H-[1,2,4]oxadiazol-5-one;

3-(1-Aminomethyl-cyclohexylmethyl)-4H-[1,2,4]oxadiazole-5-thione;

(1S-cis)3-(1-Aminomethyl-3-methyl-cyclohexylmethyl)-4H-[1,2,4]oxadiazole-5-thione;

(trans)3-(1-Aminomethyl-3,4-dimethyl-cyclopentylmethyl)-4H-[1,2,4]oxadiazole-5-thione;

(1S-cis)3-(1-Aminomethyl-3-methyl-cyclopentylmethyl)-4H-[1,2,4]oxadiazole-5-thione;

(1R-trans)3-(1-Aminomethyl-3-methyl-cyclopentylmethyl)-4H-[1,2,4]oxadiazole-5-thione;

(1R-cis)3-(1-Aminomethyl-3-methyl-cyclopentylmethyl)-4H-[1,2,4]oxadiazole-5-thione;

(1S-trans)3-(1-Aminomethyl-3-methyl-cyclopentylmethyl)-4H-[1,2,4]oxadiazole-5-thione;

(1α,3α,4α)3-(1-Aminomethyl-3,4-dimethyl-cyclopentylmethyl)-4H-[1,2,4]oxadiazole-5-thione;

(1α,3β,4β)3-(1-Aminomethyl-3,4-dimethyl-cyclopentylmethyl)-4H-[1,2,4]oxadiazole-5-thione;

(S)3-(1-Aminomethyl-3,3-dimethyl-cyclopentylmethyl)-4H-[1,2,4]oxadiazole-5-thione;

(R)3-(1-Aminomethyl-3,3-dimethyl-cyclopentylmethyl)-4H-[1,2,4]oxadiazole-5-thione;

3-(1-Aminomethyl-3,3-dimethyl-cyclobutylmethyl)-4H-[1,2,4]oxadiazole-5-thione;

C-[1-(1H-Tetrazol-5-ylmethyl)-cyclohexyl]-methylamine;

(1S-cis)C-[3-Methyl-1-(1H-tetrazol-5-ylmethyl)-cyclohexyl]-methylamine;

(trans)C-[3,4-Dimethyl-1-(1H-tetrazol-5-ylmethyl)-cyclopentyl]-methylamine;

(1S-cis)C-[3-Methyl-1(1H-tetrazol-5-ylmethyl)-cyclopentyl]-methylamine;

(1R-trans)C-[3-Methyl-1-(1H-tetrazol-5-ylmethyl)-cyclopentyl]-methylamine;

(1R-cis)C-[3-Methyl-1-(1H-tetrazol-5-ylmethyl)-cyclopentyl]-methylamine;

(1S-trans)C-[3-Methyl-1-(1H-tetrazol-5-ylmethyl)-cyclopentyl]-methylamine;

(1α,3α,4α)C-[3,4-Dimethyl-1-(1H-tetrazol-5-ylmethyl)-cyclopentyl]-methylamine;

(1α,3β,4β)C-[3,4-Dimethyl-1-(1H-tetrazol-5-ylmethyl)-cyclopentyl]-methylamine;

(S)C-[3,3-Dimethyl-1-(1H-tetrazol-5-ylmethyl)-cyclopentyl]-methylamine;

(R)C-[3,3-Dimethyl-1-(1H-tetrazol-5-ylmethyl)-cyclopentyl]-methylamine;

C-[3,3-Dimethyl-1-(1H-tetrazol-5-ylmethyl)-cyclobutyl]-methylamine;

N-[2-(1-Aminomethyl-cyclohexyl)-ethyl]-C,C,C-trifluoro-methanesulfonamide;

(1S-cis)N-[2-(1-Aminomethyl-3-methyl-cyclohexyl)-ethyl]-C,C,C-trifluoro-methanesulfonamide;

(trans)N-[2-(1-Aminomethyl-3,4-dimethyl-cyclopentyl)-ethyl]-C,C,C-trifluoro-methanesulfonamide;

(1R-cis)N-[2-(1-Aminomethyl-3-methyl-cyclopentyl)-ethyl]-C,C,C-trifluoro-methanesulfonamide;

(1S-trans)N-[2-(1-Aminomethyl-3-methyl-cyclopentyl)-ethyl]-C,C,C-trifluoro-methanesulfonamide;

(1S-cis)N-[2-(1-Aminomethyl-3-methyl-cyclopentyl)-ethyl]-C,C,C-trifluoro-methanesulfonamide;

(1R-trans)N-[2-(1-Aminomethyl-3-methyl-cyclopentyl)-ethyl]-C,C,C-trifluoro-methanesulfonamide;

(1α,3α,4α)N-[2-(1-Aminomethyl-3,4-dimethyl-cyclopentyl)-ethyl]-C,C,C-trifluoro-methanesulfonamide;

(1α,3β,4β)N-[2-(1-Aminomethyl-3,4-dimethyl-cylopentyl)-ethyl]-C,C,C-trifluoro-methanesulfonamide;

(S)N-[2-(1-Aminomethyl-3,3-dimethyl-cyclopentyl)-ethyl]-C,C,C-trifluoro-methanesulfonamide;

(R)N-[2-(1-Aminomethyl-3,3-dimethyl-cyclopentyl)-ethyl]-C,C,C-trifluoro-methanesulfonamide;

N-[2-(1-Aminomethyl-3,3-dimethyl-cyclobutyl)-ethyl]-C,C,C-trifluoro-methanesulfonamide;

3-(1-Aminomethyl-cyclohexylmethyl)-4H-[1,2,4]thiadiazol-5-one;

(1S-cis)3-(1-Aminomethyl-3-methyl-cyclohexylmethyl)-4H-[1,2,4]thiadiazol-5-one;

(trans)3-(1-Aminomethyl-3,4-dimethyl-cyclopentylmethyl)-4H-[1,2,4]thiadiazol-5-one;

(1R-cis)3-(1-Aminomethyl-3-methyl-cyclopentylmethyl)-4H-[1,2,4]thiadiazol-5-one;

(1S-trans)3-(1-Aminomethyl-3-methyl-cyclopentylmethyl)-4H-[1,2,4]thiadiazol-5-one;

(1S-cis)3-(1-Aminomethyl-3-methyl-cyclopentylmethyl)-4H-[1,2,4]thiadiazol-5-one;

(1R-trans)3-(1-Aminomethyl-3-methyl-cyclopentylmethyl)-4H-[1,2,4]thiadiazol-5-one;

(1α,3α,4α)3-(1-Aminomethyl-3,4-dimethyl-cyclopentylmethyl)-4H-[1,2,4]thiadiazol-5-one;

(1α,3β,4β)3-(1-Aminomethyl-3,4-dimethyl-cyclopentylmethyl)-4H-[1,2,4]thiadiazol-5-one;

(S)3-(1-Aminomethyl-3,3-dimethyl-cyclopentylmethyl)-4H-[1,2,4]thiadiazol-5-one;

(R)3-(1-Aminomethyl-3,3-dimethyl-cyclopentylmethyl)-4H-[1,2,4]thiadiazol-5-one;

3-(1-Aminomethyl-3,3-dimethyl-cyclobutylmethyl)-4H-[1,2,4]thiadiazol-5-one;

C-[1-(2-Oxo-2,3-dihydro-2λ⁴)-[1,2,3,5]oxathiadiazol-4-ylmethyl)-cyclohexyl]-methylamine;

(1S-cis)C-[3-Methyl-1-(2-oxo-2,3-dihydro-2λ⁴-[1,2,3,5]oxathiadiazol-4-ylmethyl)-cyclohexyl]-methylamine;

(trans)C-[3,4-Dimethyl-1-(2-oxo-2,3-dihydro-2λ⁴-[1,2,3,5]oxathiadiazol-4-ylmethyl)-cyclopentyl]-methylamine;

(1S-cis)C-[3-Methyl-1-(2-oxo-2,3-dihydro-2λ⁴-[1,2,3,5]oxathiadiazol-4-ylmethyl)-cyclopentyl]-methylamine;

(1R-trans)C-[3-Methyl-1-(2-oxo-2,3-dihydro-2λ⁴-[1,2,3,5]oxathiadiazol-4-ylmethyl)-cyclopentyl]-methylamine;

(1R-cis)C-[3-Methyl-1-(2-oxo-2,3-dihydro-2λ⁴-[1,2,3,5]oxathiadiazol-4-ylmethyl)-cyclopentyl]-methylamine;

(1S-trans)C-[3-Methyl-1-(2-oxo-2,3-dihydro-2λ⁴-[1,2,3,5]oxathiadiazol-4-ylmethyl)-cyclopentyl]-methylamine;

(1α,3α,4α)C-[3,4-Dimethyl-1-(2-oxo-2,3-dihydro-2λ⁴-[1,2,3,5]oxathiadiazol-4-ylmethyl)-cyclopentyl]-methylamine;

(1α,3β,4β)C-[3,4-Dimethyl-1-(2-oxo-2,3-dihydro-2λ⁴-[1,2,3,5]oxathiadiazol-4-ylmethyl)-cyclopentyl]-methylamine;

(S)C-[3,3-Dimethyl-1-(2-oxo-2,3-dihydro-2λ⁴-[1,2,3,5]oxathiadiazol-4-ylmethyl)-cyclopentyl]methylamine;

(R)C-[3,3-Dimethyl-1-(2-oxo-2,3-dihydro-2λ⁴-[1,2,3,5]oxathiadiazol-4-ylmethyl)-cyclopentyl]-methylamine;

C-[3,3-Dimethyl-1-(2-oxo-2,3-dihydro-2λ⁴-[1,2,3,5]oxathiadiazol-4-ylmethyl)-cyclobutyl]-methylamine;

(1-Aminomethyl-cyclohexyl)-methanesulfonamide;

(1R-trans)(1-Aminomethyl-3-methyl-cyclohexyl)-methanesulfonamide;

(trans)(1-Aminomethyl-3,4-dimethyl-cyclopentyl)-methanesulfonamide;

(1S-trans)(1-Aminomethyl-3-methyl-cyclopentyl)-methanesulfonamide;

(1R-cis)(1-Aminomethyl-3-methyl-cyclopentyl)-methanesulfonamide;

(1R-trans)(1-Aminomethyl-3-methyl-cyclopentyl)-methanesulfonamide;

(1S-cis)(1-Aminomethyl-3-methyl-cyclopentyl)-methanesulfonamide;

(1α,3β,4β)(1-Aminomethyl-3,4-dimethyl-cyclopentyl)-methanesulfonamide;

(1α,3α,4α)(1-Aminomethyl-3,4-dimethyl-cyclopentyl)-methanesulfonamide;

(R)(1-Aminomethyl-3,3-dimethyl-cyclopentyl)-methanesulfonamide;

(S)(1-Aminomethyl-3,3-dimethyl-cyclopentyl)-methanesulfonamide;

(1-Aminomethyl-3,3-dimethyl-cyclobutyl)-methanesulfonamide;

(1-Aminomethyl-cyclohexyl)-methanesulfonic acid;

(1R-trans)(1-Aminomethyl-3-methyl-cyclohexyl)-methanesulfomic acid;

(trans)(1-Aminomethyl-3,4-dimethyl-cyclopentyl)-methanesulfonic acid;

(S-trans)(1-Aminomethyl-3-methyl-cyclopentyl)-methanesulfonic acid;

(1S -cis)(1-Aminomethyl-3-methyl-cyclopentyl)-methanesulfonic acid;

(1R-trans)(1-Aminomethyl-3-methyl-cyclopentyl)-methanesulfonic acid;

(1R-cis)(1-Aminomethyl-3-methyl-cyclopentyl)-methanesulfonic acid;

(1α,3β,4β)(1-Aminomethyl-3,4-dimethyl-cyclopentyl)-methanesulfonic acid;

(1α,3α,4α)(1-Aminomethyl-3,4-dimethyl-cyclopentyl)-methanesulfonic acid;

(R)(1-Aminomethyl-3,3-dimethyl-cyclopentyl)-methanesulfonic acid;

(S)(1-Aminomethyl-3,3-dimethyl-cyclopentyl)-methanesulfonic acid;

(1-Aminomethyl-3,3-dimethyl-cyclobutyl)-methanesulfonic acid;

(1-Aminomethyl-cyclopentylmethyl)-phosphonic acid;

2-(1-Aminomethyl-cyclopentyl)-N-hydroxy-acetamide;

N-[2-(1-Aminomethyl-cyclopentyl)-ethyl]-methanesulfonamide;

3-(1-Aminomethyl-cyclopentylmethyl)-4H-[1,2,4]oxadiazol-5-one,

3-(1-Aminomethyl-cyclopentylmethyl)-4H-[1,2,4]oxadiazole-5-thione;

C-[1-(1H-Tetrazol-5-ylmethyl)-cyclopentyl]-methylamine;

N-[2-(1-Aminomethyl-cyclopentyl)-ethyl]-C,C,C-trifluoro-methanesulfonamide;

3-(1-Aminomethyl-cyclopentylmethyl)-4H-[1,2,4]thiadiazol-5-one;

C-[1-(2-Oxo-2,3-dihydro-2λ⁴-[1,2,3,5]oxathiadiazol-4-ylmethyl)-cyclopentyl]-methylamine;

(1-Aminomethyl-cyclopentyl)-methanesulfoniamide;

(1-Aminomethyl-cyclopentyl)-methanesulfonic acid;

(9-Aminomethyl-bicyclo[3.3.1]non-9-ylmethyl)-phosphonic acid;

2-(9-Aminomethyl-bicyclo[3.3.1]non-9-yl)-N-hydroxy-acetamide;

N-[2-(9-Aminomethyl-bicyclo[3.3.1]non-9-yl)-ethyl]-methanesulfonamide;

3-(9-Aminomethyl-bicyclo[3.3.1]non-9-ylmethyl)-4H-[1,2,4]oxadiazol-5-one;

3-(9-Aminomethyl-bicyclo[3.3.1]non-9-ylmethyl)-4H-[1,2,4]oxadiazole-5-thione;

C-[9-(1 H-Tetrazol-5-ylmethyl)-bicyclo[3.3.1]non-9-yl]-methylamine;

N-[2-(9-Aminomethyl-bicyclo[3.3.1]non-9-yl)-ethyl]-C,C,C-trifluoro-methanesulfonamide;

3-(9-Aminomethyl-bicyclo[3.3.1]non-9-ylmethyl)-4H-[1,2,4]thiadiazol-5-one;

C-[9-(2-Oxo-2,3-dihydro-2λ⁴-[1,2,3,5]oxathiadiazol-4-ylmethyl)-bicyclo[3.3.1]non-9-yl]-methylamine;

(9-Aminomethyl-bicyclo[3.3.1]non-9-yl)-methanesulfonamide;

(9-Aminomethyl-bicyclo[3.3.1]non-9-yl)-methanesulfonic acid;

(2-Aminomethyl-adamantan-2-ylmethyl)-phosphonic acid;

2-(2-Aminomethyl-adamantan-2-yl)-N-hydroxy-acetamide;

N-[2-(2-Aminomethyl-adamantan-2-yl)-ethyl]-methanesulfonamide;

3-(2-Aminomethyl-adamantan-2-ylmethyl)-4H-[1,2,4]oxadiazol-5-one;

3-(2-Aminomethyl-adamantan-2-ylmethyl)-4H-[1,2,4]oxadiazole-5-thione;

C-[2-(1H-Tetrazol-5-ylmethyl)-adamantan-2-yl]-methylamine;

N-[2-(2-Aminomethyl-adamantan-2-yl)-ethyl]-C,C,C-trifluoro-methanesulfonamide;

3-(2-Aminomethyl-adamantan-2-ylmethyl)-4H-[1,2,4]thiadiazol-5-one;

C-[2-(2-Oxo-2,3-dihydro-2λ⁴-[1,2,3,5]oxathiadiazol-4-ylmethyl)-adamantan-2-yl]-methylamine;

(2-Aminomethyl-adamantan-2-yl)-methanesulfonamide;

(2-Aminomethyl-adamantan-2-yl)-methanesulfonic acid;

(1-Aminomethyl-cycloheptylmethyl)-phosphonic acid;

2-(1-Aminomethyl-cycloheptyl)-N-hydroxy-acetamide;

N-[2-(1-Aminomethyl-cycloheptyl)-ethyl]-methanesulfonamide;

3-(1-Aminomethyl-cycloheptylmethyl)-4H-[1,2,4]oxadiazol-5-one;

3-(1-Aminomethyl-cycloheptylmethyl)4H-[1,2,4]oxadiazole-5-thione;

C-[1-(1H-Tetrazol-5-ylmethyl)-cycloheptyl]-methylamine;

N-[2-(1-Aminomethyl-cycloheptyl)-ethyl]-C,C,C-trifluoro-methanesulfonamide;

C-[1-(2-Oxo-2,3-dihydro-2 14-[1,2,3,5]oxathiadiazol-4-ylmethyl)-cycloheptyl]-methylamine;

(1-Aminomethyl-cycloheptyl)-methanesulfonamide; and

(1-Aminomethyl-cycloheptyl)-methanesulfonic acid.

Another preferred embodiment of the present invention includes a compound of Formula III, IIIC, IIIF, IIIG, or IIIH, wherein preferred compounds are those wherein R is a sulfonamide selected from —NHSO₂R¹⁵ or —SO₂NHR¹⁵ wherein R¹⁵ is straight or branched alkyl or trifluoromethyl.

Another preferred embodiment of the present invention includes a compound of Formula III, IIIC, IIIF, IIIG, or IIIH, wherein especially preferred is N-[2-(1-aminomethyl-cyclohexyl)-ethyl]-methanesulfonamide.

Another preferred embodiment of the present invention includes a compound of Formula III, IIIC, IIIF, IIIG, or IIIH, wherein other preferred compounds are those wherein R is a phosphonic acid, —PO₃H₂.

Another preferred embodiment of the present invention includes a compound of Formula III, IIIC, IIIF, IIIG, or IIIH, wherein especially preferred are (1-aminomethyl-cyclohexylmethyl)-phosphonic acid and (2-aminomethyl-4-methyl-pentyl)-phosphonic acid.

Another preferred embodiment of the present invention includes a compound of Formula III, IIIC, IIIF, IIIG, or IIIH, wherein other preferred compounds are those wherein R is a heterocycle selected from:

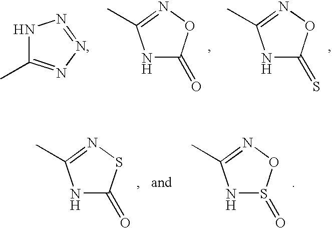

Another preferred embodiment of the present invention includes a compound of Formula III, IIIC, IIIF, IIIG, or IIIH, wherein especially preferred are C-[1-(1H-tetrazol-5-ylmethyl)cyclohexyl]-methylamine and 4-methyl-2-(1H-tetrazol-5-ylmethyl)-pentylamine.

An especially preferred embodiment of the present invention includes a compound of Formula III wherein:

m is an integer of from 0 to 2;

p is an integer of 2; and

R is or

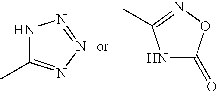

Still more preferred is an embodiment of the present invention which includes a compound of Formula III named 3-(1-aminomethyl-cyclohexylmethyl)-4H-[1,2,4]oxadiazol-5-one, and pharmaceutically acceptable salts thereof. Still more preferred is 3-(1-aminomethyl-cyclohexylmethyl)-4H-[1,2,4]oxadiazol-5-one hydrochloride.

Another preferred embodiment of the present invention includes a compound of Formula IV

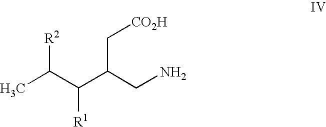

or a pharmaceutically acceptable salt thereof wherein:

R¹ is hydrogen, straight or branched alkyl of from 1 to 6 carbon atoms or phenyl;

R² is straight or branched alkyl of from 1 to 8 carbon atoms,

straight or branched alkenyl of from 2 to 8 carbon atoms,

cycloalkyl of from 3 to 7 carbon atoms,

alkoxy of from 1 to 6 carbon atoms,

alkylcycloalkyl,

alkylalkoxy,

alkyl OH,

alkylphenyl,

alkylphenoxy,

phenyl or substituted phenyl; and

R¹ is straight or branched alkyl of from 1 to 6 carbon atoms or phenyl when R² is methyl.

Preferred is an embodiment including a compound of Formula IV wherein R¹ is hydrogen, and R² is alkyl.

Another preferred embodiment includes a compound of Formula IV wherein R¹ is methyl, and R² is alkyl.

Still another preferred embodiment includes a compound of Formula IV wherein R¹ is methyl, and R² is methyl or ethyl.

Especially preferred is an embodiment including a compound of Formula IV selected from:

3-Aminomethyl-5-methylheptanoic acid;

3-Aminomethyl-5-methyl-octanoic acid;

3-Aminomethyl-5-methyl-nonanoic acid;

3-Aminomethyl-5-methyl-decanoic acid;

3-Aminomethyl-5-methyl-undecanoic acid;

3-Aminomethyl-5-methyl-dodecanoic acid;

3-Aminomethyl-5-methyl-tridecanoic acid;

3-Aminomethyl-5-cyclopropyl-hexanoic acid;

3-Aminomethyl-5-cyclobutyl-hexanoic acid;

3-Aminomethyl-5-cyclopentyl-hexanoic acid;

3-Aminomethyl-5-cyclohexyl-hexanoic acid;

3-Aminomethyl-5-trifluoromethyl-hexanoic acid;

3-Aminomethyl-5-phenyl-hexanoic acid;

3-Aminomethyl-5-(2-chlorophenyl)-hexanoic acid;

3-Aminomethyl-5-(3-chlorophenyl)-hexanoic acid;

3-Aminomethyl-5-(4-chlorophenyl)-hexanoic acid;

3-Aminomethyl-5-(2-methoxyphenyl)-hexanoic acid;

3-Aminomethyl-5-(3-methoxyphenyl)-hexanoic acid;

3-Aminomethyl-5-(4-methoxyphenyl)-hexanoic acid; and

3-Aminomethyl-5-(phenylmethyl)-hexanoic acid.

Another especially preferred embodiment includes compounds selected from:

(3R,4S)-3-Aminomethyl-4,5-dimethyl-hexanoic acid;

3-Aminomethyl-4,5-dimethyl-hexanoic acid;

(3R,4S)-3-Aminomethyl-4,5-dimethyl-hexanoic acid MP;

(3S,4S)-3-Aminomethyl-4,5-dimethyl-hexanoic acid;

(3R,4R)-3-Aminomethyl-4,5-dimethyl-hexanoic acid MP;

3-Aminomethyl-4-isopropyl-hexanoic acid;

3-Aminomethyl-4-isopropyl-heptanoic acid;

3-Aminomethyl-4-isopropyl-octanoic acid;

3-Aminomethyl-4-isopropyl-nonanoic acid;

3-Aminomethyl-4-isopropyl-decanoic acid; and

3-Aminomethyl-4-phenyl-5-methyl-hexanoic acid.

Another preferred embodiment includes compounds selected from:

(3S,5S)-3-Aminomethyl-5-methoxy-hexanoic acid;

(3S,5S)-3-Aminomethyl-5-ethoxy-hexanoic acid;

(3S,5S)-3-Aminomethyl-5-propoxy-hexanoic acid;

(3S,5S)-3-Aminomethyl-5-isopropoxy-hexanoic acid;

(3S,5S)-3-Aminomethyl-5-tert-butoxy-hexanoic acid;

(3S,5S)-3-Aminomethyl-5-fluoromethoxy-hexanoic acid;

(3S,5S)-3-Aminomethyl-5-(2-fluoro-ethoxy)-hexanoic acid;

(3S,5S)-3-Aminomethyl-5-(3,3,3-trifluoro-propoxy)-hexanoic acid;

(3S,5S)-3-Aminomethyl-5-phenoxy-hexanoic acid;

(3S,5S)-3-Aminomethyl-5-(4-chloro-phenoxy)-hexanoic acid;

(3S,5S)-3-Aminomethyl-5-(3-chloro-phenoxy)-hexanoic acid;

(3S,5S)-3-Aminomethyl-5-(2-chloro-phenoxy)-hexanoic acid;

(3S,5S)-3-Aminomethyl-5-(4-fluoro-phenoxy)-hexanoic acid;

(3S,5S)-3-Aminomethyl-5-(3-fluoro-phenoxy)-hexanoic acid;

(3S,5S)-3-Aminomethyl-5-(2-fluoro-phenoxy)-hexanoic acid;

(3S,5S)-3-Aminomethyl-5-(4-methoxy-phenoxy)-hexanoic acid;

(3S,5S)-3-Aminomethyl-5-(3-methoxy-phenoxy)-hexanoic acid;

(3S,5S)-3-Aminomethyl-5-(2-methoxy-phenoxy)-hexanoic acid;

(3S,5S)-3-Aminomethyl-5-(4-nitro-phenoxy)-hexanoic acid;

(3S,5S)-3-Aminomethyl-5-(3-nitro-phenoxy)-hexanoic acid;

(3S,5S)-3-Aminomethyl-5-(2-nitro-phenoxy)-hexanoic acid;

(3S,5S)-3-Aminomethyl-6-hydroxy-5-methyl-hexanoic acid;

(3S,5S)-3-Aminomethyl-6-methoxy-5-methyl-hexanoic acid;

(3S,5S)-3-Aminomethyl-6-ethoxy-5-methyl-hexanoic acid;

(3S,5S)-3-Aminomethyl-5-methyl-6-propoxy-hexanoic acid;

(3S,5S)-3-Aminomethyl-6-isopropoxy-5-methyl-hexanoic acid;

(3S,5S)-3-Aminomethyl-6-tert-butoxy-5-methyl-hexanoic acid;

(3S,5S)-3-Aminomethyl-6-fluoromethoxy-5-methyl-hexanoic acid;

(3S,5S)-3-Aminomethyl-6-(2-fluoro-ethoxy)-5-methyl-hexanoic acid;

(3S,5S)-3-Aminomethyl-5-methyl-6-(3,3,3-trifluoro-propoxy)-hexanoic acid;

(3S,5S)-3-Aminomethyl-5-methyl-6-phenoxy-hexanoic acid;

(3S,5S)-3-Aminomethyl-6-(4-chloro-phenoxy)-5-methyl-hexanoic acid;

(3S,5S)-3-Aminomethyl-6-(3-chloro-phenoxy)-5-methyl-hexanoic acid;

(3S,5S)-3-Aminomethyl-6-(2-chloro-phenoxy)-5-methyl-hexanoic acid;

(3S,5S)-3-Aminomethyl-6-(4-fluoro-phenoxy)-5-methyl-hexanoic acid;

(3S,5S)-3-Aminomethyl-6-(3-fluoro-phenoxy)-5-methyl-hexanoic acid;

(3S,5S)-3-Aminomethyl-6-(2-fluoro-phenoxy)-5-methyl-hexanoic acid;

(3S,5S)-3-Aminomethyl-6-(4-methoxy-phenoxy)-5-methyl-hexanoic acid;

(3S,5S)-3-Aminomethyl-6-(3-methoxy-phenoxy)-5-methyl-hexanoic acid;

(3S,5S)-3-Aminomethyl-6-(2-methoxy-phenoxy)-5-methyl-hexanoic acid;

(3S,5S)-3-Aminomethyl-5-methyl 6-(4-trifluoromethyl-phenoxy)-hexanoic acid;

(3S,5S)-3-Aminomethyl-5-methyl 6-(3-trifluoromethyl-phenoxy)-hexanoic acid;

(3S,5S)-3-Aminomethyl-5-methyl 6-(2-trifluoromethyl-phenoxy)-hexanoic acid;

(3S,5S)-3-Aminomethyl-5-methyl 6-(4-nitro-phenoxy)-hexanoic acid;

(3S,5S)-3-Aminomethyl-5-methyl 6-(3-nitro-phenoxy)-hexanoic acid;

(3S,5S)-3-Aminomethyl-5-methyl 6-(2-nitro-phenoxy)-hexanoic acid;

(3S,5S)-3-Aminomethyl-6-benzyloxy-5-methyl-hexanoic acid;

(3S,5S)-3-Aminomethyl-7-hydroxy-5-methyl-heptanoic acid;

(3S,5S)-3-Aminomethyl-7-methoxy-5-methyl-heptanoic acid;

(3S,5S)-3-Aminomethyl-7-ethoxy-5-methyl-heptanoic acid;

(3S,5S)-3-Aminomethyl-5-methyl-7-propoxy-heptanoic acid;

(3S,5S)-3-Aminomethyl-7-isopropoxy-5-methyl-heptanoic acid;

(3S,5S)-3-Aminomethyl-7-tert-butoxy-5-methyl-heptanoic acid;

(3S,5S)-3-Aminomethyl-7-fluoromethoxy-5-methyl-heptanoic acid;

(3S,5S)-3-Aminomethyl-7-(2-fluoro-ethoxy)-5-methyl-heptanoic acid;

(3S,5S)-3-Aminomethyl-5-methyl-7-(3,3,3-trifluoro-propoxy)-heptanoic acid;

(3S,5S)-3-Aminomethyl-7-benzyloxy-5-methyl-heptanoic acid;

(3S,5S)-3-Aminomethyl-5-methyl-7-phenoxy-heptanoic acid;

(3S,5S)-3-Aminomethyl-7-(4-chloro-phenoxy)-5-methyl-heptanoic acid;

(3S,5 S)-3-Aminomethyl-7-(3-chloro-phenoxy)-5-methyl-heptanoic acid;

(3S,5)-3-Aminomethyl-7-(2-chloro-phenoxy)-5-methyl-heptanoic acid;

(3S,5S)-3-Aminomethyl-7-(4-fluoro-phenoxy)-5-methyl-heptanoic acid;

(3S,5S)-3-Aminomethyl-7-(3-fluoro-phenoxy)-5-methyl-heptanoic acid;

(3S,5S)-3-Aminomethyl-7-(2-fluoro-phenoxy)-5-methyl-heptanoic acid;

(3S,5S)-3-Aminomethyl-7-(4-methoxy-phenoxy)-5-methyl-heptanoic acid;

(3S,5S)-3-Aininomethyl-7-(3-methoxy-phenoxy)-5-methyl-heptanoic acid;

(3S,5S)-3-Aminomethyl-7-(2-methoxy-phenoxy)-5-methyl-heptanoic acid;

(3S,5S)-3-Aminomethyl-5-methyl-7-(4-trifluoromethyl-phenoxy)-heptanoic acid;

(3S,5S)-3-Aminomethyl-5-methyl-7-(3-trifluoromethyl-phenoxy)-heptanoic acid;

(3S,5S)-3-Aminomethyl-5-methyl-7-(2-trifluoromethyl-phenoxy)-heptanoic acid;

(3S,5S)-3-Aminomethyl-5-methyl-7-(4-nitro-phenoxy)-heptanoic acid;

(3S,5S)-3-Aminomethyl-5-methyl-7-(3-nitro-phenoxy)-heptanoic acid;

(3S,5S)-3-Aminomethyl-5-methyl-7-(2-nitro-phenoxy)-heptanoic acid;

(3S,5S)-3-Aminomethyl-5-methyl-6-phenyl-hexanoic acid;

(3S,5S)-3-Aminomethyl-6-(4-chloro-phenyl)-5-methyl-hexanoic acid;

(3S,5S)-3-Aminomethyl-6-(3-chloro-phenyl)-5-methyl-hexanoic acid;

(3S,5S)-3-Aminomethyl-6-(2-chloro-phenyl)-5-methyl-hexanoic acid;

(3S,5S)-3-Aminomethyl-6-(4-methoxy-phenyl)-5-methyl-hexanoic acid;

(3S,5S)-3-Aminomethyl-6-(3-methoxy-phenyl)-5-methyl-hexanoic acid;

(3S,5S)-3-Aminomethyl-6-(2-methoxy-phenyl)-5-methyl-hexanoic acid;

(3S,5S)-3-Aminomethyl-6-(4-fluoro-phenyl)-5-methyl-hexanoic acid;

(3S,5S)-3-Aminomethyl-6-(3-fluoro-phenyl)-5-methyl-hexanoic acid;

(3S,5S)-3-Aminomethyl-6-(2-fluoro-phenyl)-5-methyl-hexanoic acid;

(3S,5R)-3-Aminomethyl-5-methyl-7-phenyl-heptanoic acid;

(3S,5R)-3-Aminomethyl-7-(4-chloro-phenyl)-5-methyl-heptanoic acid;

(3S,5R)-3-Aminomethyl-7-(3-chloro-phenyl)-5-methyl-heptanoic acid;

(3S,5R)-3-Aminomethyl-7-(2-chloro-phenyl)-5-methyl-heptanoic acid;

(3S,5R)-3-Aminomethyl-7-(4-methoxy-phenyl)-5-methyl-heptanoic acid;

(3S,5R)-3-Aminomethyl-7-(3-methoxy-phenyl)-5-methyl-heptanoic acid;

(3S,5R)-3-Aminomethyl-7-(2-methoxy-phenyl)-5-methyl-heptanoic acid;

(3S,5R)-3-Aminomethyl-7-(4-fluoro-phenyl)-5-methyl-heptanoic acid;

(3S,5R)-3-Aminomethyl-7-(3-fluoro-phenyl)-5-methyl-heptanoic acid;

(3S,5R)-3-Aminomethyl-7-(2-fluoro-phenyl)-5-methyl-heptanoic acid;

(3S,5R)-3-Aminomethyl-5-methyl-oct-7-enoic acid;

(3S,5R)-3-Aminomethyl-5-methyl-non-8-enoic acid;

(E)-(3S,5S)-3-Aminomethyl-5-methyl-oct-6-enoic acid;

(Z)-(3S,5S)-3-Aminomethyl-5-methyl-oct-6-enoic acid;

(Z)-(3S,5S)-3-Aminomethyl-5-methyl-non-6-enoic acid;

(E)-(3S,5S)-3-Aminomethyl-5-methyl-non-6-enoic acid;

(E)-(3S,5R)-3-Aminomethyl-5-methyl-non-7-enoic acid;

(Z)-(3S,5R)-3-Aminomethyl-5-methyl-non-7-enoic acid;

(Z)-(3S,5R)-3-Aminomethyl-5-methyl-dec-7-enoic acid;

(E)-(3S,5R)-3-Aminomethyl-5-methyl-undec-7-enoic acid;

(3S,5S)-3-Aminomethyl-5,6,6-trimethyl-heptanoic acid;

(3S,5S)-3-Aminomethyl-5,6-dimethyl-heptanoic acid;

(3S,5S)-3-Aminomethyl-5-cyclopropyl-hexanoic acid;

(3S,5S)-3-Aminomethyl-5-cyclobutyl-hexanoic acid;

(3S,5S)-3-Amnomethyl-5-cyclopentyl-hexanoic acid; and

(3S,5S)-3-Aminomethyl-5-cyclohexyl-hexanoic acid.

Still another more preferred embodiment includes a compound of Formula IV selected from:

(3S,5R)-3-Aminomethyl-5-methyl-heptanoic acid;

(3S,5R)-3-Aminomethyl-5-methyl-octanoic acid;

(3S,5R)-3-Aminomethyl-5-methyl-nonanoic acid;

(3S,5R)-3-Aminomethyl-5-methyl-decanoic acid;

(3S,5R)-3-Aminomethyl-5-methyl-undecanoic acid;

(3S,5R)-3-Aminomethyl-5-methyl-dodecanoic acid;

(3S,5R)-3-Aminomethyl-5,9-dimethyl-decanoic acid;

(3S,5R)-3-Aminomethyl-5,7-dimethyl-octanoic acid;

(3S,5R)-3-Aminomethyl-5,8-dimethyl-nonanoic acid;

(3S,5R)-3-Aminomethyl-6-cyclopropyl-5-methyl-hexanoic acid;

(3S,5R)-3-Aminomethyl-6-cyclobutyl-5-methyl-hexanoic acid;

(3S,5R)-3-Aminomethyl-6-cyclopentyl-5-methyl-hexanoic acid;

(3S,5R)-3-Aminomethyl-6-cyclohexyl-5-methyl-hexanoic acid;

(3S,5R)-3-Aminomethyl-7-cyclopropyl-5-methyl-heptanoic acid;

(3S,5R)-3-Aminomethyl-7-cyclobutyl-5-methyl-heptanoic acid;

(3S,5R)-3-Aminomethyl-7-cyclopentyl-5-methyl-heptanoic acid;

(3S,5R)-3-Aminomethyl-7-cyclohexyl-5-methyl-heptanoic acid;

(3S,5R)-3-Aminomethyl-8-cyclopropyl-5-methyl-octanoic acid;

(3S,5R)-3-Aminomethyl-8-cyclobutyl-5-methyl-octanoic acid;

(3S,5R)-3-Aminomethyl-8-cyclopentyl-5-methyl-octanoic acid;

(3S,5R)-3-Aminomethyl-8-cyclohexyl-5-methyl-octanoic acid;

(3S,5S)-3-Aminomethyl-6-fluoro-5-methyl-hexanoic acid;

(3S,5S)-3-Aminomethyl-7-fluoro-5-methyl-heptanoic acid;

(3S,5R)-3-Aminomethyl-8-fluoro-5-methyl-octanoic acid;

(3S,5R)-3-Aminomethyl-9-fluoro-5-methyl-nonanoic acid;

(3S,5S)-3-Aminomethyl-7,7,7-trifluoro-5-methyl-heptanoic acid;

(3S,5R)-3-Aminomethyl-8,8,8-trifluoro-5-methyl-octanoic acid;

(3S,5R)-3-Aminomethyl-5-methyl-8-phenyl-octanoic acid;

(3S,5S)-3-Aminomethyl-5-methyl-6-phenyl-hexanoic acid; and

(3S,5R)-3-Aminomethyl-5-methyl-7-phenyl-heptanoic acid.

Preferred is a combination of the invention comprising an endothelin antagonist. that antagonizes both the ET_{A} and ET_{B} receptor subtypes.

Preferred is a combination of the invention comprising an endothelin antagonist that is selective for the ET_{A} receptor subtype.

Preferred is a combination of the invention comprising an endothelin antagonist that is selective for the ET_{B} receptor subtype.

Preferred is a combination of the invention comprising an endothelin antagonist which is a compound of Formula V

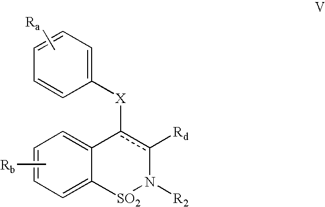

or a pharmaceutically acceptable acid addition or base salt thereof wherein:

R₂ is H,

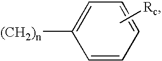

alkyl of from 1 to 7 carbons, (CH₂)ₙ-cycloalkyl of from 3 to 8 carbons;

Rₐ and R_{c} are each 1 to 5 substituents and R_{b} is from 1 to 4 substituents independently selected from:

hydrogen,

alkyl of from 1 to 7 carbons,

alkenyl of from 2 to 7 carbons,

alkynyl of from 2 to 7 carbons,

cycloalkyl of from 3 to 8 carbons, phenyl,

C(O)-phenyl,

methylenedioxy,

ethylenedioxy,

OR,

NRR₁,

SR₁,

NO₂,

N₃,

COR,

CO₂R,

Cl,

Br,

F,

CONRR₁,

SO₂NRR₁,

SO₂R,

CN,

CF₃,

CF₂CF₃,

CHO,

OCOR,

B(OH)₂,

NH(CH₂)ₚCO₂R,

S(CH₂)ₚCO₂R,

O(CH₂)ₚCO₂R,

O(CH₂)ₚOR,

NH(CH₂)ₚOR,

S(CH₂)ₚOR, or

wherein R and R₁ are each independently selected from

hydrogen,

alkyl of from 1 to 6 carbon atoms,

alkenyl of from 2 to 7 carbon atoms,

alkynyl of from 2 to 7 carbon atoms,

cycloalkyl of from 3 to 8 carbon atoms,

phenyl or benzyl wherein the phenyl or benzyl ring is substituted by one or more hydrogen, methoxy, and methylenedioxy substituents;

R_{d} is H, CO₂R, SO₃R, PO₃R, B(OH)₂, CONRR₁, SO₂NRR₁, C(O)NHSO₂R₁,

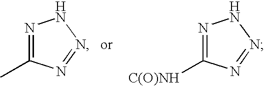

n is an integer of from 0 to 2;

p is an integer of from 1 to 4;

--- indicates a single or double bond; and

X is (CH₂)ₙ, O, NR, or S(O)ₙ.

Also preferred is a combination of the invention comprising an endothelin antagonist which is a compound of Formula VI

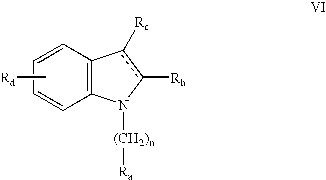

wherein

--- denotes an optional bond;

n is0to4;

Rₐ is hydrogen, alkyl of 1-4 carbon atoms or cycloalkyl, phenyl or naphthyl, in which the phenyl or naphthyl group is substituted by methylenedioxy and further unsubstituted or substituted by one or more substituents selected from the group consisting of halogen, alkyl of 1-6 carbon atoms, OR, NRR¹, SR, NO₂, N₃, COR, CO₂R, CONRR¹, SO₂NRR¹, SO₂R, CN, CF₃, CF₂CF₃, CHO, OCOCH₃, B(OH)₂, phenyl, NH(CH₂)ₘCO₂R, S(CH₂)ₘCO₂R, O(CH₂)ₘCO₂R, O(CH₂)ₘOR, NH(CH₂)ₘOR and S(CH₂)ₘOR, in which m is 1, 2 or 3, and R and R¹ are each independently hydrogen, alkyl of 1-4 carbon atoms, phenyl or benzyl;

R_{b} is hydrogen, CO₂R²,

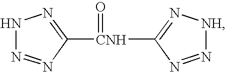

SO₃R, PO₃H, B(OH)₂, CONR¹R², SO₂NR¹R², or

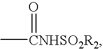

in which R¹ is as defined above and R² is hydrogen, alkyl of 1-6 carbon atoms, CF₃, —CF₂CF₃, phenyl or benzyl in which phenyl or the phenyl portion of the benzyl group is unsubstituted or substituted by one or more substituents as defined above;

R_{c} is S(O)ₚ-phenyl, in which p is 0, 1 or 2, and phenyl is unsubstituted or substituted by one or more substituents selected from the group consisting of halogen, OR, NRR¹, SR, NO₂, N₃, COR, CO₂R, CONRR¹, SO₂NRR¹, SO₂R, CN, CF₃, CF₂CF₃, CHO, OCOCH₃, B(OH)₂, methylenedioxy, NH(CH₂)ₘCO₂R, S(CH₂)ₘCO₂R, O(CH₂)ₘCO₂R, O(CH₂)ₘOR, NH(CH₂)ₘOR and S(CH₂)ₘOR, in which m, R and R¹ are as defined above, and

R_{d} is 1 to 4 independent substituents selected from hydrogen, alkyl of 1-7 carbon atoms, alkenyl of 2-7 carbon atoms, alkynyl of 2-7 carbon atoms, cycloalkyl, phenyl, C(O)-phenyl, X(CH₂)ₙ-phenyl, X-(CH₂)ₙ-naphthyl, in which X is 0, NH or S(O)ₚ, methylenedioxy, OR, NRR¹, SR, NO₂, N₃, COR, CO₂R, CONRR¹, SO₂NRR¹, SO₂R, CN, CF₃, CF₂CF₃, CHO, OCOCH₃, B(OH)₂, phenyl, NH(CH₂)ₘCO₂R, S(CH₂)ₘCO₂R, O(CH₂)ₘCO₂R, O(CH₂)ₘOR, NH(CH₂)ₘOR, S(CH₂)ₘOR, in which m is 1, 2 or 3 and R and R¹ are each independently hydrogen, alkyl of 1-4 carbon atoms, phenyl or benzyl and where n and p are as defined above and phenyl is unsubstituted or substituted as defined above, or a pharmaceutically acceptable acid addition or base salt thereof.

More preferred is a combination of the invention comprising an endothelin antagonist which is a compound selected from:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, potassium salt;

4-Benzo[1,3]dioxol-5-yl-2-methyl-1,1-dioxo-1,2-dihydro-1λ⁶-benzo[e][1,2]thiazine-3-carboxylic acid;

4-Benzo[1,3]dioxol-5-yl-2-benzo[1,3]dioxol-5-ylmethyl-1,1-dioxo-1,2-dihydro-1λ⁶-benzo[e][1,2]thiazine-3-carboxylic acid;

4-Benzo[1,3]dioxol-5-yl-2-benzyl-1,1-dioxo-1,2-dihydro-1λ⁶-benzo[e][1,2]thiazine-3-carboxylic acid;

4-Benzo[1,3]dioxol-5-yl-2-(4-methoxy-benzyl)-1,1-dioxo-1,2-dihydro-1λ⁶-benzo[e][1,2]thiazine-3-carboxylic acid;

4-Benzo[1,3]dioxol-5-yl-1,1-dioxo-2-(3,4,5-trimethoxy-benzyl)-1,2-dihydro-1λ⁶-benzo[e][1,2]thiazine-3-carboxylic acid;

4-Benzo[1,3]dioxol-5-yl-2-(2-carboxymethoxy-4-methoxy-benzyl)-1,1-dioxo-1,2-dihydro-1λ⁶-benzo[e][1,2]thiazine-3-carboxylic acid;

4-Benzo[1,3]dioxol-5-yl-2-(6-chloro-benzo[1,3]dioxol-5-ylmethyl)-1,1-dioxo-1,2-dihydro-1λ⁶-benzo[e][1,2]thiazine-3-carboxylic acid;

4-Benzo[1,3]dioxol-5-yl-2-(7-methoxy-benzo[1,3]dioxol-5-ylmethyl)-1,1-dioxo-1,2-dihydro-1λ⁶-benzo[e][1,2]thiazine-3-carboxylic acid;

2-Benzo[1,3]dioxol-5-ylmethyl-4-(3,4-dimethoxy-phenyl)-1,1-dioxo-1,2-dihydro-1λ⁶-benzo[e][1,2]thiazine-3-carboxylic acid;

2-Benzo[1,3]dioxol-5-ylmethyl-1,1-dioxo-4-(3,4,5-trimethoxy-phenyl)-1,2-dihydro-1λ⁶-benzo[e][1,2]thiazine-3-carboxylic acid;

N-(4-Benzo[1,3]dioxol-5-yl-2-benzo[1,3]dioxol-5-ylmethyl-1,1-dioxo-1,2-dihydro-1λ⁶-benzo[e][1,2]thiazine-3-carbonyl)-benzenesulfonamide;

2-Benzo[1,3]dioxol-5-ylmethyl-4-(3-methoxy-phenyl)-1,1-dioxo-1,2-dihydro-1λ⁶-benzo[e][1,2]thiazine-3-carboxylic acid;

4-Benzo[1,3]dioxol-5-yl-2-benzo[1,3]dioxol-5-ylmethyl-6,7-dimethoxy-1,1-dioxo-1,2-dihydro-1λ⁶-benzo[e][1,2]thiazine-3-carboxylic acid;

4-Benzo[1,3]dioxol-5-yl-2-benzo[1,3]dioxol-5-ylmethyl-6-methoxy-1,1-dioxo-1,2-dihydro-1λ⁶-benzo[e][1,2]thiazine-3-carboxylic acid;

8-Benzo[1,3]dioxol-5-yl-6-benzo[1,3]dioxol-5-ylmethyl-5,5-dioxo-5,6-dihydro-1,3-dioxa-5λ⁶-thia-6-aza-cyclopenta[b]naphthalene-7-carboxylic acid;

4-(Benzo[1,3]dioxol-5-ylsulfanyl)-2-methyl-1,1-dioxo-1,2-dihydro-1λ⁶-benzo[e][1,2]thiazine-3-carboxylic acid;

2-Benzo[1,3]dioxol-5-ylmethyl-4-(benzo[1,3]dioxol-5-ylsulfanyl)-1,1-dioxo-1,2-dihydro-1λ⁶-benzo[e][1,2]thiazine-3-carboxylic acid;

4-(Benzo[1,3]dioxol-5-ylsulfanyl)-2-benzyl-1,1-dioxo-1,2-dihydro-1λ⁶-benzo[e][1,2]thiazine-3-carboxylic acid;

4-(Benzo[1,3]dioxol-5-ylsulfanyl)-2-(4-methoxy-benzyl)-1,1-dioxo-1,2-dihydro-1λ⁶-benzo[e][1,2]thiazine-3-carboxylic acid;

4-(Benzo[1,3]dioxol-5-ylsulfanyl)-1,1-dioxo-2-(3,4,5-trimethoxy-benzyl)-1,2-dihydro-1λ⁶-benzo[e][1,2]thiazine-3-carboxylic acid;

4-(Benzo[1,3]dioxol-5-ylsulfanyl)-2-(carboxymethoxy-4-methoxy-benzyl)-1,1-dioxo-1,2-dihydro-1λ⁶-benzo[e][1,2]thiazine-3-carboxylic acid;

4-(Benzo[1,3]dioxol-5-ylsulfanyl)-2-(6-chloro-benzo[1,3]dioxol-5-ylmethyl)-1,1-dioxo-1,2-dihydro-1λ⁶-benzo[e][1,2]thiazine-3-carboxylic acid;

4-(Benzo[1,3]dioxol-5-ylsulfanyl)-2-(7-methoxy-benzo[1,3]dioxol-5-ylmethyl)-1,1-dioxo-1,2-dihydro-1λ⁶-benzo[e][1,2]thiazine-3-carboxylic acid;

2-Benzo[1,3]dioxol-5-ylmethyl4-(3,4-dimethoxy-phenylsulfanyl)-1,1-dioxo-1,2-dihydro-1λ⁶-benzo[e][1,2]thiazine-3-carboxylic acid;

2-Benzo[1,3]dioxol-5-ylmethyl-1,1-dioxo-4-(3,4,5-trimethoxy-phenylsulfanyl)-1,2-dihydro-1λ⁶-benzo[e][1,2]thiazine-3-carboxylic acid;

N-(4-Benzo[1,3]dioxol-5-ylsulfanyl-2-benzo[1,3]dioxol-5-ylmethyl-1,1-dioxo-1,2-dihydro-1λ⁶-benzo[e][1,2]thiazine-3-carbonyl)-benzenesulfonamide;

2-Benzo[1,3]dioxol-5-ylmethyl-4-(3-methoxy-phenylsulfanyl)-1,1-dioxo-1,2-dihydro-1λ⁶-benzo[e][1,2]thiazine-3-carboxylic acid;

2-Benzo[1,3]dioxol-5-ylmethyl-4-(benzo[1,3]dioxol-5-ylsulfanyl)-6,7-dimethoxy-1,1-dioxo-1,2-dihydro-1λ⁶-benzo[e][1,2]thiazine-3-carboxylic acid;

2-Benzo[1,3]dioxol-5-ylmethyl-4-(benzo[1,3]dioxol-5-ylsulfanyl)-6-methoxy-1,1-dioxo-1,2-dihydro-1λ⁶-benzo[e][1,2]thiazine-3-carboxylic acid;

6-Benzo[1,3]dioxol-5-ylmethyl-8-(benzo[1,3]dioxol-5-ylsulfanyl)-5,5-dioxo-5,6-dihydro-1,3-dioxa-5λ⁶-thia-6-aza-cyclopenta[b]naphthalene-7-carboxylic acid;

4-(Benzo[1,3]dioxol-5-ylsulfanyl)-2-isobutyl-1,1-dioxo-1,2-dihydro-1λ⁶-benzo[e][1,2]thiazine-3-carboxylic acid;

2-Benzo[1,3]dioxol-5-ylmethyl-4-(benzo[1,3]dioxol-5-ylsulfanyl)-7-methoxy-1,1-dioxo-1,2-dihydro-1λ⁶-benzo[e][1,2]thiazine-3-carboxylic acid;

2-Benzo[1,3]dioxol-5-ylmethyl-4-(2,3-dihydro-benzo[1,4]dioxin-6-ylsulfanyl)-1,1-dioxo-1,2-dihydro-1λ⁶-benzo[e][1,2]thiazine-3-carboxylic acid;

4-(Benzo[1,3]dioxol-5-ylsulfanyl)-2-(2,3-dihydro-benzo[1,4]dioxin-6-ylmethyl)-1,1-dioxo-1,2-dihydro-1λ⁶-benzo[e][1,2]thiazine-3-carboxylic acid;

4-(Benzo[1,3]dioxol-5-ylsulfanyl)-2-cyclohexylmethyl-1,1-dioxo-1,2-dihydro-1λ⁶-benzo[e][1,2]thiazine-3-carboxylic acid;

2-Benzo[1,3]dioxol-5-yl-4-(benzo[1,3]dioxol-5-ylsulfanyl)-1,1-dioxo-1,2-dihydro-1λ⁶-benzo[e][1,2]thiazine-3-carboxylic acid;

2-Benzo[1,3]dioxol-5-yl-4-(benzo[1,3]dioxol-5-ylsulfanyl)-6,7-dimethoxy-1,1-dioxo-1,2-dihydro-1λ⁶-benzo[e][1,2]thiazine-3-carboxylic acid;

2,4-Bis-benzo[1,3]dioxol-5-yl-1,1-dioxo-1,2-dihydro-1λ⁶-benzo[e][1,2]thiazine-3-carboxylic acid;

2,4-Bis-benzo[1,3]dioxol-5-yl-6,7-dimethoxy-1,1-dioxo-1,2-dihydro-1λ⁶-benzo[e][1,2]thiazine-3-carboxylic acid;

4-Benzo[1,3]dioxol-5-yl-2-(2-chloro-benzyl)-1,1-dioxo-1,2-dihydro-1λ⁶-benzo[e][1,2]thiazine-3-carboxylic acid;

2-Benzo[1,3]dioxol-5-ylmethyl-4-(4-chloro-2,6-dimethoxy-phenyl)-1,1-dioxo-1,2-dihydro-1λ⁶-benzo[e][1,2]thiazine-3-carboxylic acid;

4-(Benzo[1,3]dioxol-5-ylsulfanyl)-2-(2-chloro-benzyl)-1,1-dioxo-1,2-dihydro-1λ⁶-benzo[e][1,2]thiazine-3-carboxylic acid;

2-Benzo[1,3]dioxol-5-ylmethyl-4-(4-chloro-2,6-dimethoxy-phenylsulfanyl)-1,1-dioxo-1,2-dihydro-1λ⁶-benzo[e][1,2]thiazine-3-carboxylic acid;

4-(Benzo[1,3]dioxol-5-yl)-2-isobutyl-1,1-dioxo-1,2-dihydro-1λ⁶-benzo[e][1,2]thiazine-3-carboxylic acid;

2-Benzo[1,3]dioxol-5-ylmethyl-4-(benzo[1,3]dioxol-5-yl)-7-methoxy-1,1-dioxo-1,2-dihydro-1λ⁶-benzo[e][1,2]thiazine-3-carboxylic acid;

2-Benzo[1,3]dioxol-5-ylmethyl-4-(2,3-dihydro-benzo[1,4]dioxin-6-yl)-1,1-dioxo-1,2-dihydro-1λ⁶-benzo[e][1,2]thiazine-3-carboxylic acid;

4-(Benzo[1,3]dioxol-5-yl)-2-(2,3-dihydro-benzo[1,4]dioxin-6-ylmethyl)-1,1-dioxo-1,2-dihydro-1λ⁶-benzo[e][1,2]thiazine-3-carboxylic acid;

4-(Benzo[1,3]dioxol-5-yl)-2-cyclohexylmethyl-1,1-dioxo-1,2-dihydro-1λ⁶-benzo[e][1,2]thiazine-3-carboxylic acid;

1-Benzo[1,3]dioxol-5-yl-3-phenylsulfanyl-1H-indole-2-carboxylic acid;

1-Benzo[1,3]dioxol-5-ylmethyl-5,6-dimethoxy-3-(3-methoxy-phenylsulfanyl)-1H-indole-2-carboxylic acid;

1-Benzo[1,3]dioxol-5-ylmethyl-3-(3-methoxy-phenylsulfanyl)-1H-indole-2-carboxylic acid;

1-Benzyl-3-(3-methoxy-phenylsulfanyl)-1H-indole-2-carboxylic acid;

1-Benzo[1,3]dioxol-5-ylmethyl-3-(benzo[1,3]dioxol-5-ylsulfanyl)-1H-indole-2-carboxylic acid;

5-Benzo[1,3]dioxol-5-ylmethyl-7-(3-methoxy-phenylsulfanyl)-5H-[1,3]dioxolo[4,5-f]indole-6-carboxylic acid;

5-(7-Methoxy-benzo[1,3]dioxol-5-ylmethyl)-7-(3-methoxy-phenylsulfanyl)-5H-[1,3]dioxolo[4,5-f]indole-6-carboxylic acid;

5-Benzo[1,3]dioxol-5-ylmethyl-7-(3,4-dimethoxy-phenylsulfanyl)-5H-[1,3]dioxolo[4,5-f]indole-6-carboxylic acid;

7-(3,4-Dimethoxy-phenylsulfanyl)-5-(7-methoxy-benzo[1,3]dioxol-5-ylmethyl)-5H-[1,3]dioxolo[4,5-f]indole-6-carboxylic acid;

1-Benzo[1,3]dioxol-5-ylmethyl-3-(3-methoxy-phenylsulfanyl)-6-propoxy-1H-indole-2-carboxylic acid;

5,6-Dimethoxy-1-(7-methoxy-benzo[1,3]dioxol-5-ylmethyl)-3-(3-methoxy-phenylsulfanyl)-1H-indole-2-carboxylic acid; and

5,6-Dimethoxy-1-(4-methoxy-benzyl)-3-(3-methoxy-phenylsulfanyl)-1H-indole-2-carboxylic acid;

1-Benzo[1,3]dioxol-5-ylmethyl-5-benzyloxy-6-methoxy-3-(3-methoxy-phenylsulfanyl)-1H-indole-2-carboxylic acid;

1-Benzo[1,3]dioxol-5-ylmethyl-5,6-dimethoxy-3-(3,4,5-trimethoxy-phenylsulfanyl)-1H-indole-2-carboxylic acid;

1-Benzo[1,3]dioxol-5-ylmethyl-3-(benzo[1,3]dioxol-5-ylsulfanyl)-6-benzyloxy-5-methoxy-1H-indole-2-carboxylic acid;

1-(2-Carboxymethoxy-4-methoxy-benzyl)-5,6-dimethoxy-3-(3-methoxy-phenylsulfanyl)-1H-indole-2-carboxylic acid;

1-Benzo[1,3]dioxol-5-ylmethyl-3-(benzo[1,3]dioxol-5-ylsulfanyl)-5,6-dimethoxy-1H-indole-2-carboxylic acid;

1-Benzo[1,3]dioxol-5-ylmethyl-3-(3,4,5-trimethoxy-phenylsulfanyl)-6-benzyloxy-5-methoxy-1H-indole-2-carboxylic acid;

5-Benzo[1,3]dioxol-5-ylmethyl-7-(3,4,5-trimethoxy-phenylsulfanyl)-5H-[1,3]dioxolo[4,5-flindole-6-carboxylic acid; and

5-Benzo[1,3]dioxol-5-ylmethyl-7-(benzo[1,3]dioxol-5-ylsulfanyl)-5H-[1,3]dioxolo[4,5-f]indole-6-carboxylic acid.

[2S-(2Alpha,3beta,4alpha)]-4-(1,3-benzodioxol-5-yl)-1-[2-(dibutylamino)-2-oxoethyl]-2-(4-methoxyphenyl)-3-pyrrolidinecarboxylic acid;

3-Pyrrolidinecarboxylic acid, 4-(1,3-benzodioxol-5-yl)-1-[2-(dibutylamino)-2-oxoethyl]-2-(4-methoxyphenyl)-, sodium salt, (2R,3R,4S)-rel-;

N-[6-(2-Hydroxyethoxy)-5-(2-methoxyphenoxy)-2-[2-(1H-tetrazol-5-yl)-4-pyridinyl]-4-pyrimidinyl]-5-(1-methylethyl)-2-pyridinesulfonamide;

5-(Dimethylamino)-N-(3,4-dimethyl-5-isoxazolyl)-1-naphthalenesulfonamide;

4-(1,1-Dimethylethyl)-N-[6-(2-hydroxyethoxy)-5-(2-methoxyphenoxy)[2,2′-bipyrimidin]-4-yl]benzenesulfonamide;

Benzenesulfonamide, 4-(1,1-dimethyl)-N-[6-(2-hydroxyethoxy)-5-(3-methoxyphenoxy)-4-pyrimidinyl]-;

[N-cis-2,6-Dimethylpiperidinocarbonyl-L-.gamma.-methylleucyl-D-1-methoxycarbonyltryptophanyl-D-norleucine];

[5S-[5Alpha,6beta,7alpha(R*)]]-5-(1,3-benzodioxol-5-yl)-2-butyl-7-[2-(2-carboxypropyl)-4-methoxyphenyl]-6,7-dihydro-5H-cyclopenta[b]pyridine-6-carboxylic acid;

(5S-(5Alpha,6beta,7alpha(R*)))-2-butyl-5-(1,3-benzodiox-ol-5-yl)-7-((2-carboxypropyl)-4-methoxyphenyl)-6-dihydro-5H-cyclopenta(b)pyridine-6-carboxylic acid (J-104120);

(5S-(5Alpha,6beta,7alpha(R*)))-5-(1,3-benzodioxol-5-yl)-2-butyl-7-(2-(2-carboxypropyl)-4-methoxyphenyl)-6,7-dihydro-5H-cyclopenta[b]pyridine-6-carboxylic acid (L-753037);

(S)-Alpha-[(4,6-Dimethoxy-2-pyrimidinyl)oxy]-beta-methoxy-beta-phenyl benzenepropanoic acid;

Alpha-((4,6-dimethoxy-2-pyrimidinyl)oxy)-beta-methoxy-beta-phenyl-benzenepropanoic acid (LU-127043);

2-(4,6-Dimethoxypyrimidin-2-yloxy)-3-ethoxy-3,3-diphenylpropionic acid;

N-[6-(2-Hydroxyethoxy)-5-(2-methoxyphenoxy)-2-[2-(1H-tetrazol-5-yl)-4-pyridinyl]-4-pyrimidinyl]-5-methyl-2-pyridinesulfonamide;

2-Pyridinesulfonamide, N-[6-(2-hydroxyethoxy)-5-(2-methoxyphenoxy)-2-[2-(1H-tetrazol-5-yI)-4-pyridinyl]-4-pyrimidinyl]-5-methyl-;

27-O-3-[2-(3-Carboxy-acryloylamino)-5-hydroxyphenyl]-acryloyloxy myricerone;

N-[6-[2-[(5-Bromo-2-pyrimidinyl)oxy]ethoxy]-5-(4-methylphenyl)-4-pyrimidinyl]-4-(2-hydroxy-1,1-dimethylethyl)benzenesulfonamide monosodium;

4-tert-Butyl-N-(5-(4-methylphenyl)-6-(2-(5-(3-thienyl)pyrimidin-2-yloxy)ethoxy)pyrimidin-4-yl)-benzenesulfonamide (T-0115);

Cyclo[4-oxo-4-(4-phenyl-1-piperazinyl)-L-2-aminobutanoyl-L-alpha-aspartyl-D-2-(2-thienyl)glycyl-L-leucyl-D-tryptophyl-D-alpha-aspartyl]disodium salt;

N-(4-Chloro-3-methyl-5-isoxazolyl)-2-[(6-methyl-1,3-benzodioxol-5-yl)acetyl]-3-thiophenesulfonamide;

2R-(4-Methoxyphenyl)-4S-(1,3-benzodioxol-5-yl)-1-(N,N-di(n-butyl)aminocarbonyl-methyl)-pyrrolidine-3R-carboxylic acid (ABT-627);

3-Thiophenesulfonamide, N-(4-chloro-3-methyl-5-isoxazolyl)-2-[(6-methyl-1,3-benzodioxol-5-yl)acetyl]- (IPI-1040);

3-Thiophenesulfonamide, N-(4-chloro-3-methyl-5-isoxazolyl)-2-[(6-methyl-1,3-benzodioxol-5-yl)acetyl]- (IPI-1251); and

3-Thiophenesulfonamide, N-(4-chloro-3-methyl-5-isoxazolyl)-2-[(6-methyl-1,3-benzodioxol-5-yl)acetyl]- (TBC-11241).

More preferred is a combination of the present invention wherein the endothelin receptor antagonist is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, or a pharmaceutically acceptable salt thereof.

Still more preferred is a combination of the invention wherein the endothelin receptor antagonist is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt.

Also more preferred is a combination of the present invention wherein the endothelin receptor antagonist is 4-(3,5-dimethyl-phenyl)-1,1-dioxo-2-(2-trifluoromethyl-phenyl)-1,2-dihydro-1λ⁶-benzo[e][1,2]thiazine-3-carboxylic acid, or a pharmaceutically acceptable salt thereof.

Still more preferred is a combination of the present invention wherein the endothelin receptor antagonist is 4-(3,5-dimethyl-phenyl)-1,1-dioxo-2-(2-trifluoromethyl-phenyl)-1,2-dihydro-1λ⁶-benzo[e][1,2]thiazine-3-carboxylic acid.

The compound named 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt is the compound of formula (1) shown below.

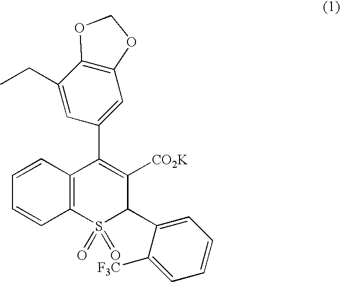

This compound is also known as 4-(7-ethyl-benzo[1,3]dioxol-5-yl)-1,1-dioxo-2-(2-trifluoromethyl-phenyl)-1,2-dihydro-1λ⁶-benzo[e][1,2]thiazine-3-carboxylic acid, potassium salt.

Also more preferred is a novel combination of the invention wherein the endothelin antagonist is selected from:

2R-(4-methoxyphenyl)-4S-(1,3-benzodioxol-5-yl)-1-(N,N-di(n-butyl)aminocarbonyl-methyl)-pyrrolidine-3R-carboxylic acid (ABT-627).

In a preferred embodiment of the present invention, the combination is comprised of compounds of Formula I, II, or III. In a more preferred embodiment of the present invention, the combination will contain the compound gabapentin, pregabalin, or 3-(1-aminomethyl-cyclohexylmethyl)-4H-[1,2,4]oxadiazol-5-one hydrochloride.

Also preferred is a novel combination of the invention wherein the antiepileptic compounds having pain alleviating properties is gabapentin and pregabalin.

More preferred is a novel combination of the invention wherein the endothelin receptor antagonist is selected from:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;

4-(7-Ethyl-1,3-benzodiozol-5-yl)-2-[2-(trifluoromethyl)phenyl]-2-1,2-dihydro-1,2-benzothiazine-3-carboxylic acid, 1,1-dioxide;

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt; and

the antiepileptic compound having pain alleviating properties is selected from gabapentin, pregabalin, and 3-(1-aminomethyl-cyclohexylmethyl)-4H-[1,2,4]oxadiazol-5-one hydrochloride.

Also more preferred is a novel combination of the invention wherein the endothelin receptor antagonist is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and the analgesic is an antiepileptic compound having pain alleviating properties named gabapentin.

Also more preferred is a novel combination of the invention wherein the endothelin receptor antagonist is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and the analgesic is an antiepileptic compound having pain alleviating properties named pregabalin.

Also more preferred is a novel combination of the invention wherein the endothelin receptor antagonist is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and the analgesic is an antiepileptic compound having pain alleviating properties named 3-(1-aminomethyl-cyclohexylmethyl)-4H-[1,2,4]oxadiazol-5-one hydrochloride.

Preferred is a novel combination of a pain alleviating effective amount of an endothelin receptor antagonist or a pharmaceutically acceptable salt thereof and from 1 to 3 compounds independently selected from the group consisting of antiepileptic compounds having pain alleviating properties and analgesics, and pharmaceutically acceptable salts thereof, wherein the analgesic is an opioid analgesic.

More preferred is a novel combination of the invention wherein the analgesic is an opioid analgesic selected from:

Codeine;

Morphine;

Hydromorphone;

Levorphanol;

Methadone;

Oxycodone;

Hydrocodone;

Pentazocine;

Nalbuphine;

Butorphanol;

Hydromorphone; and

Naloxone.

Still more preferred is a novel combination of the invention wherein the endothelin receptor antagonist is selected from:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt

and the analgesic is an opioid analgesic selected from:

Codeine;

Morphine;

Hydromorphone;

Levorphanol;

Methadone;

Oxycodone;

Hydrocodone;

Pentazocine;

Nalbuphine;

Butorphanol;

Hydromorphone; and

Naloxone.

Preferred is a novel combination of a pain alleviating effective amount of an endothelin receptor antagonist or a pharmaceutically acceptable salt thereof and from 1 to 3 compounds independently selected from the group consisting of antiepileptic compounds having pain alleviating properties and analgesics, and pharmaceutically acceptable salts thereof, wherein the analgesic is a nonopioid analgesic.

Preferred is a novel combination of the invention wherein the analgesic is an NSAID.

More preferred is a novel combination of the invention wherein the analgesic is an NSAID selected from:

Naproxen;

Naproxen sodium;

Ibuprofen;

Acetaminophen;

Aspirin;

Sulindac;

Tolmetin;

Piroxicam;

Mefenamic acid;

Phenylbutazone;

Fenoprofen;

Ketoprofen;

Suprofen;

Diflunisal;

Celecoxib;

Meloxicam; and

(Z)-5-[[3,5-Bis(1,1-dimethylethyl)-4-hydroxyphenyl]methylene]-2-imino-4-thiazolidinone methanesulfonate (1:1).

Still more preferred is a novel combination of the invention wherein the endothelin receptor antagonist is selected from:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt

and the analgesic is an NSAID nonopioid analgesic selected from:

Naproxen;

Naproxen sodium;

Ibuprofen;

Acetaminophen;

Aspirin;

Sulindac;

Tolmetin;

Piroxicam;

Mefenamic acid;

Phenylbutazone;

Fenoprofen;

Ketoprofen;

Suprofen;

Diflunisal;

Celecoxib;

Meloxicam; and

(Z)-5-[[3,5-Bis(1,1-dimethylethyl)-4-hydroxyphenyl]methylene]-2-imino-4-thiazolidinone methanesulfonate (1:1).

Preferred is a novel combination of the invention wherein the analgesic is an NMDA receptor antagonist.

More preferred is a novel combination of the invention wherein the analgesic is an NMDA receptor antagonist selected from:

1H-Indole-2-carboxylic acid, 4,6-dichloro-3-[3-oxo-3-(phenylamino)-1-propenyl]-, (E)- (GV-150526);

1-Piperidineethanol, .alpha.-(4-hydroxyphenyl)-.beta.-methyl-4-(phenylmethyl)-(Ifenprodil);

ACEA 1168; and

(1S,2S)-1-(4-Hydroxyphenyl)-2-(4-hydroxy-4-phenylpiperidine)-1-propanol.

Still more preferred is a novel combination of the invention wherein the endothelin receptor antagonist is selected from:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt

and the analgesic is an NMDA receptor antagonist nonopioid analgesic selected from:

1H-Indole-2-carboxylic acid, 4,6-dichloro-3-[3-oxo-3-(phenylamino)-1-propenyl]-, (E)- (GV-150526);

1-Piperidineethanol, .alpha.-(4-hydroxyphenyl)-.beta.-methyl-4-(phenylmethyl)- (Ifenprodil);

ACEA 1168; and

(1S,2S)-1-(4-Hydroxyphenyl)-2-(4-hydroxy-4-phenylpiperidine)-1-propanol.

Preferred is a novel combination of the invention wherein the analgesic is an NK₁ receptor antagonist.

More preferred is a novel combination of the invention wherein the analgesic is an NK₁ receptor antagonist named:

[2-(1H-Indol-3-yl)-1-methyl-1-(1-phenyl-ethylcarbamoyl)-ethyl]-carbamic acid benzofuran-2-ylmethyl ester.

Still more preferred is a novel combination of the invention wherein the endothelin receptor antagonist is selected from:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid,

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, and

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, potassium salt;

and the analgesic is an NK₁ receptor antagonist nonopioid analgesic named:

[2-(1H-Indol-3-yl)-1-methyl-1-(1-phenyl-ethylearbamoyl)-ethyl]-carbamic acid benzofuran-2-ylmethyl ester.

Preferred is a novel combination of a pain alleviating effective amount of an endothelin receptor antagonist or a pharmaceutically acceptable salt thereof and from 1 to 3 compounds independently selected from the group consisting of antiepileptic compounds having pain alleviating properties and analgesics, and pharmaceutically acceptable salts thereof, wherein one compound is an antiepileptic compound having pain alleviating properties and one compound is an analgesic.

More preferred is a novel combination of the invention wherein one compound is an antiepileptic compound having pain alleviating properties and one compound is an opioid analgesic.

Still more preferred is a novel combination of the invention wherein the endothelin receptor antagonist is selected from:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid,

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt;

the antiepileptic compound having pain alleviating properties is selected from gabapentin, pregabalin, and 3-(1-aminomethyl-cyclohexylmethyl)4H-[1,2,4]oxadiazol-5-one hydrochloride; and

the analgesic is a nonopioid analgesic selected from:

Codeine;

Morphine;

Hydromorphone;

Levorphanol;

Methadone;

Oxycodone;

Hydrocodone;

Pentazocine;

Nalbuphine;

Butorphanol;

Hydromorphone; and

Naloxone.

More preferred is a novel combination of the invention wherein one compound is an antiepileptic compound having pain alleviating properties and one compound is a nonopioid analgesic.

Still more preferred is a novel combination of the invention wherein the endothelin receptor antagonist is selected from:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt

the antiepileptic compound having pain alleviating properties is selected from gabapentin, pregabalin, and 3-(1-aminomethyl-cyclohexylmethyl)-4H-[1,2,4]oxadiazol-5-one hydrochloride, and

the analgesic is a nonopioid analgesic selected from:

Naproxen;

Naproxen sodium;

Ibuprofen;

Acetaminophen;

Aspirin;

Sulindac;

Tolmetin;.

Piroxicam;

Mefenamic acid;

Phenylbutazone;

Fenoprofen;

Ketoprofen;

Suprofen;

Diflunisal;

Celecoxib;

Meloxicam;

(Z)-5-[[3,5-Bis(1,1-dimethylethyl)-4-hydroxyphenyl]methylene]-2-imino-4-thiazolidinone methanesulfonate (1:1);

[2-(1H-Indol-3-yl)-1-methyl-1-(1-phenyl-ethylcarbamoyl)-ethyl]-carbainic acid benzofuran-2-ylmethyl ester;

1H-Indole-2-arboxylic acid, 4,6-dichloro-3-[3-oxo-3-(phenylamino)-1-propenyl]-, (E)- (GV-150526);

1-Piperidineethanol, .alpha.-(4-hydroxyphenyl)-.beta.-methyl-4-(phenylmethyl)- (Ifenprodil);

ACEA 1168; and

(1S,2S)-1-(4-Hydroxyphenyl)-2-(4-hydroxy-4-phenylpiperidine)-1-propanol.

Preferred is a novel combination of a pain alleviating effective amount of an endothelin receptor antagonist or a pharmaceutically acceptable salt thereof and from 1 to 3 compounds independently selected from the group consisting of antiepileptic compounds having pain alleviating properties and analgesics, and pharmaceutically acceptable salts thereof, wherein one compound is an opioid analgesic and one compound is a nonopioid analgesic.

Still more preferred is a novel combination of the invention wherein the endothelin receptor antagonist is selected from:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and

4-(7-Ethyl-1,3-benzodioxol-5-yl)-2-[2-(trifluoromethyl)pbenyl]-2H-1,2-dihydro-1,2-benzothiazine-3-carboxylic acid, 1,1-dioxide, potassium salt

the opioid analgesic is selected from

Codeine;

Morphine;

Hydromorphone;

Levorphanol;

Methadone;

Oxycodone;

Hydrocodone;

Pentazocine;

Nalbuphine;

Butorphanol;

Hydromorphone; and

Naloxone;

and the nonopioid analgesic is selected from:

Naproxen;

Naproxen sodium;

Ibuprofen;

Acetaminophen;

Aspirin;

Sulindac;

Tolmetin;

Piroxicam;

Mefenamic acid;

Phenylbutazone;

Fenoprofen;

Ketoprofen;

Suprofen;

Diflunisal;

Celecoxib;

Meloxicam;

(Z)-5-[[3,5-Bis(1,1-dimethylethyl)-4-hydroxyphenyl]methylene]-2-imino-4-thiazolidinone methanesulfonate (1:1);

[2-(1H-Indol-3-yl)-1-methyl-1-(1-phenyl-ethylcarbamoyl)-ethyl]-carbamic acid benzofuran-2-ylmethyl ester;

1H-Indole-2-carboxylic acid, 4,6-dichloro-3-[3-oxo-3-(phenylamino)-1-propenyl]-, (E)- (GV-150526);

1-Piperidineethanol, .alpha.-(4-hydroxyphenyl)-.beta.-methyl-4-(phenylmethyl)- (Ifenprodil);

ACEA 1168; and

(1S,2S)-1-(4-Hydroxyphenyl)-2-(4-hydroxy-4-phenylpiperidine)-1-propanol.

Another embodiment of the present invention is a pharmaceutical composition, comprising a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, and a pharmaceutically acceptable carrier, diluent, or excipient.

Another embodiment of the present invention is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof.

Preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the pain being treated is selected from the group consisting of: centrally mediated pain, peripherally mediated pain, structural pain, soft tissue pain, injury-related pain, progressive disease-related pain, and neuropathic pain.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the pain being treated is diabetic peripheral neuropathy.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is gabapentin, or a pharmaceutically acceptable salt thereof.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable.salts thereof, wherein the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is pregabalin, or a pharmaceutically acceptable salt thereof.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is 3-(1-aminomethyl-cyclohexylmethyl)-4H-[1,2,4]oxadiazol-5-one hydrochloride, or a pharmaceutically acceptable salt thereof.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the pain being treated is diabetic peripheral neuropathy and the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is gabapentin, or a pharmaceutically acceptable salt thereof.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the pain being treated is diabetic peripheral neuropathy and the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is pregabalin, or a pharmaceutically acceptable salt thereof.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the pain being treated is diabetic peripheral neuropathy and the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is 3-(1-aminomethyl-cyclohexylmethyl)-4H-[1,2,4]oxadiazol-5-one hydrochloride, or a pharmaceutically acceptable salt thereof.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the combination administered comprises an endothelin receptor antagonist which is 4-(3,5-dimethyl-phenyl)-1,1-dioxo-2-(2-trifluoromethylphenyl)-1,2-dihydro-1λ⁶-benzo[e][1,2]thiazine-3-carboxylic acid, and an antiepileptic compound having pain alleviating properties which is gabapentin, or a pharmaceutically acceptable salt thereof.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the combination administered comprises an endothelin receptor antagonist which is 4-(3,5-dimethyl-phenyl)-1,1-dioxo-2-(2-trifluoromethylphenyl)-1,2-dihydro-1λ⁶-benzo[e][1,2]thiazine-3-carboxylic acid, and an antiepileptic compound having pain alleviating properties which is pregabalin, or a pharmaceutically acceptable salt thereof.

Also preferred is a method of treating pain in a mammal suffering therefrom, comprising administering a pain alleviating effective amount of a combination of an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, wherein the combination administered comprises an endothelin receptor antagonist which is 4-(3,5-dimethyl-phenyl)-1,1-dioxo-2-(2-trifluoromethylphenyl)-1,2-dihydro-1λ⁶-benzo[e][1,2]thiazine-3-carboxylic acid, and an antiepileptic compound having pain alleviating properties which is 3-(1-aminomethyl-cyclohexylmethyl)-4H-[1,2,4]oxadiazol-5-one hydrochloride, or a pharmaceutically acceptable salt thereof.

Another embodiment of the present invention is a method of treating pain in a mammal suffering therefrom, comprising administering a pain alleviating effective amount of a pharmaceutical composition which comprises an endothelin antagonist, or a pharmaceutically acceptable salt thereof, and from 1 to 3 compounds independently selected from the group consisting of antiepileptic compounds having pain alleviating properties and analgesics, and pharmaceutically acceptable salts thereof, and a pharmaceutically acceptable carrier, diluent, or excipient.

### BRIEF DESCRIPTION OF THE DRAWINGS

FIG. 1 illustrates dose-response curves for gabapentin administered alone and the compound of formula (1) administered alone, expressed as the Pain Withdrawal Threshold (PWT) in grams of force of von Frey hairs measured at 1-hour postdose (the time point at which peak activity for each compound is observed), versus oral dose of compound administered in milligrams of compound per kilogram of rat body weight. The compound of formula (1) is described below.

FIG. 2 illustrates the dose-response curves for a combination of gabapentin and the compound of formula (1) administered at a 1:1 ratio (weight/weight), respectively, expressed as the PWT in grams of force of von Frey hairs versus total oral dose of the combination administered in total milligrams of the combination per kilogram of body weight, and the dose-response curves of FIG. 1.

FIG. 3 illustrates the dose-response curves for a combination of gabapentin and the compound of formula (1) administered at a 3:1 ratio (weight/weight), respectively, expressed as the PWT in grams of force of von Frey hairs versus total oral dose of the combination administered in total milligrams of the combination per kilogram of body weight, and the dose-response curves of FIG. 1.

FIG. 4 illustrates the dose-response curves for a combination of gabapentin and the compound of formula (1) administered at a 10:1 ratio (weight/weight), respectively, expressed as the PWT in grams of force of von Frey hairs versus total oral dose of the combination administered in total milligrams of the combination per kilogram of body weight, and the dose-response curves of FIG. 1.

### DETAILED DESCRIPTION OF THE INVENTION

The present invention is directed to novel combinations effective for alleviating pain comprising a pain alleviating effective amount of an endothelin receptor antagonist or a pharmaceutically acceptable salt thereof, and one or two compounds selected from the group consisting of antiepileptic compounds having pain alleviating properties and analgesics, and to methods of treating or alleviating pain comprising administering effective amounts of said combinations to a mammal, including a human, in need of said treatment. The invention also is directed to pharmaceutical compositions comprising said combinations.

In one embodiment of the present invention, a single endothelin receptor antagonist is combined with a single compound selected from the group consisting of antiepileptic compounds having pain alleviating properties, opioid analgesics, or nonopioid analgesics including NSAIDs, NMDA receptor antagonists, and NK₁ receptor antagonists. Preferred combinations include, but are not limited to, ET antagonist/opioid, ET antagonist/morphine, ET antagonist/hydrocodone, ET antagonist/oxycodone, ET antagonist/ibuprofen, ET antagonist/naproxen, ET antagonist/acetaminophen, ET antagonist/gabapentin, ET antagonist/pregabalin, and ET antagonist/3-(1-aminomethyl-cyclohexylmethyl)-4H-[1,2,4]oxadiazol-5-one hydrochloride.

In another embodiment of the present invention, a single endothelin antagonist is combined with two or three, preferably two, compounds selected from the group consisting of NSAIDs, opioid analgesics, NMDA receptor antagonists, NK₁ receptor antagonists, or combinations thereof. While any endothelin antagonist disclosed herein can be combined with any two or three compounds selected from NSAID, opioid analgesic, NMDA receptor antagonists, NKI receptor antagonists, or combinations thereof, the preferred ET receptor antagonists are:

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/morphine/naproxen;

4-(7-Ethyl -1,3-benzodiozol-5-yl)-2-[2-(trifluoromethyl)phenyl]-2H-1,2-dihydro-1,2-benzothiazine-3-carboxylic acid, 1,1-dioxide/morphine/naproxen; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/morphine/naproxen.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/opioid/NSAID;

4-(7-Ethyl-1,3-benzodiozol-5-yl)-2-1,2-(trifluoromethyl)phenyl]-2H-1,2-dihydro-1,2-benzothiazine-3-carboxylic acid, 1,1-dioxide/opioid/NSAID; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/opioid/NSAID.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/morphine/ibuprofen;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide/morphine/ibuprofen; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/morphine/ibuprofen.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/hydrocodone/acetaminophen;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide/hydrocodone/acetaminophen; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/hydrocodone/acetaminophen.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/oxycodone/acetaminophen;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide/oxycodone/acetaminophen; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/oxycodone/acetaminophen.

Preferred combinations include, but are not limited to,

4-(3,5-Dimethyl-phenyl)-1,1-dioxo-2-(2-trifluoromethyl-phenyl)-1,2-ihydro-1λ⁶-benzo[e][1,2]thiazine-3-carboxylic acid/gabapentin or pregabalin/morphine;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide/gabapentin; and pregabalinlmorphine; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/gabapentin, or pregabalin/morphine.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/gabapentin or pregabalin/hydrocodone;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide/gabapentin or pregabalin/hydrocodone; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/gabapentin or pregabalin/hydrocodone.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/gabapentin or pregabalin/oxycodone;

4-(7-Ethyl-1,3-benzodiozol-5-yI)-2-[2-(trifluoromethyl)phenyl]-2H-1,2-dihydro-1,2-benzothiazine-3-carboxylic acid, 1,1-dioxide/gabapentin or pregabalin/oxycodone; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/gabapentin or pregabalin/oxycodone.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/gabapentin or pregabalin/acetaminophen;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide/gabapentin or pregabalin/acetaminophen; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/gabapentin or pregabalin/acetaminophen.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/gabapentin or pregabalin/ibuprofen;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide/gabapentin or pregabalin/ibuprofen; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/gabapentin or pregabalin/ibuprofen.

Preferred combinations include, but are not limited to,

4-(3,5-Dimethyl-phenyl)-1,1-dioxo-2-(2-trifluoromethyl-phenyl)-1,2-dihydro-1λ⁶-benzo[e][1,2]thiazine-3-carboxylic acid/gabapentin or pregabalin/naproxen;

4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide/gabapentin or pregabalin/naproxen; and

4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt/gabapentin or pregabalin/naproxen.

In another embodiment of the present invention, two or more antiepileptic compounds having pain alleviating properties are combined with an endothelin receptor antagonist and optionally NSAIDs, NMDA receptor antagonists, or NK₁ receptor antagonists, or combinations thereof. While any antiepileptics herein can be combined with any endothelin receptor antagonist and optionally with one or more compounds selected from NSAIDs, NMDA receptor antagonists, or NK₁ receptor antagonists, or combinations thereof, the preferred antiepileptic compounds having pain alleviating properties are chosen from the compounds of Formulas I, II, and III, and the preferred ET antagonists are chosen from the compounds of Formulas V and VI. Preferred combinations include, but are not limited to, gabapentin/pregabalin/ET antagonist, gabapentin/pregabalin/ ET antagonist/NSAID, gabapentin/pregabalin/naproxen/ET antagonist, and gabapentin/pregabalin/ET antagonist/opioid.

Any of the combinations disclosed herein can be used for treatment. The types of treatable pain experienced by mammals is varied and known to medical practitioners. Nonlimiting examples of mammalian pain include centrally mediated pain, peripherally mediated pain, structural or soft tissue injury related pain, progressive disease related pain (i.e., oncology) and neuropathic pain states, all of which would include both acute (i.e., acute injury or trauma, pre- and postsurgical, headache such as a migraine), chronic (i.e., neuropathic pain conditions such diabetic peripheral neuropathy and posthepatic neuralgia), and inflammatory condition (i.e., osteo- or rheumatoid arthritis, sequela to acute injury or trauma) pain states.

Nonlimiting examples of mammalian pain being treated are centrally mediated pain, peripherally mediated pain, structural or soft tissue pain, injury-related pain, progressive disease-related pain, or neuropathic pain. Pain states include both acute (e.g., acute injury or trauma, pre- and postsurgical, headache such as migraine), chronic (e.g., pain from cancer, neuropathic pain conditions such as diabetic peripheral neuropathy and posthepatic neuralgia), and inflammatory conditions (e.g., osteo- or rheumatoid arthritis, sequela to acute injury or trauma).

The term “mammal” includes a human, dog, cat, cow, sheep, pig, and horse.

As used herein, the phrase “effective amount” means the amount of a combination of the present invention that is effective for alleviating pain in a mammal suffering therefrom. The phrase “therapeutically effective amount,” as used herein, means the same as the phrase “effective amount.”

As used herein, the term “co-administration” is meant to include the administration of endothelin receptor antagonists before, during, or after administration of compounds selected from the group consisting of antiepileptics that demonstrate pain alleviating properties such as gabapentin and pregabalin, opioid analgesics, or nonopioid analgesics including NSAIDs, NMDA receptor antagonists, or NK₁ receptor antagonists.

One advantage of using the novel combinations described herein is the reduced severity and/or frequency of pain. Another potential advantage is the overall improvement in pain control, which can include a reduction in the dosage and unwanted side effects in comparison to using higher doses of a single agent treatment to achieve a similar therapeutic effect.

The present invention is valuable because combination approaches to pain alleviation take advantage of the unique properties and mechanisms of action of two or more different drugs. As discussed above, endothelin antagonists alleviate pain by a novel mechanism of action. An endothelin antagonist, an antiepileptic compound having pain alleviating properties, and an analgesic each independently contribute to pain alleviating activity by distinct mechanisms of action, so a combination of an endothelin antagonist with one or more drugs selected from the group consisting of antiepileptic compound having pain alleviating properties and analgesics will be more effective at the same doses than any single drug administered by itself (i.e., show a synergistic effect). Also, administration of combinations may allow the use of smaller doses of each individual drug to achieve a certain level of pain alleviation comparable to higher doses of a single drug, and thereby reduce or avoid side effects connected with administration of higher doses.

The phrase “endothelin receptor antagonist” as used in this invention may be used interchangeably with the phrases endothelin antagonist or ET antagonist and are taken to mean the same. A preferred endothelin receptor antagonist is 4-(7-ethyl-1,3-benzodiozol-5-yl)-2-[2-(trifluoromethyl)phenyl]-2H-1,2-dihydro-1,2-benzothiazine-3-carboxylic acid, 1,1-dioxide, and pharmaceutically acceptable salts thereof. A preferred pharmaceutically acceptable salt thereof is the potassium salt. Another preferred endothelin receptor antagonist is 4-(3,5-dimethyl-phenyl)-1,1-dioxo-2-(2-trifluoromethyl-phenyl)-1,2-dihydro-1λ⁶-benzo[e][1,2]-thiazine-3-carboxylic acid, and pharmaceutically acceptable salts thereof.

Analgesics used in this invention can be, for example, opioid analgesics or nonopioid analgesic compounds.

Nonopioid analgesics are generally defined to be those compounds that relieve pain without binding to opioid receptors. Nonlimiting examples of nonopioid analgesics include NSAIDs such as acetaminophen, NMDA receptor antagonists, and NK₁ receptor antagonists. Nonopioid analgesics are typically nonaddictive.

Nonlimiting examples of clinically validated analgesics include the following:

Opiate mu receptor agonists;

Cyclooxygenase-2 (COX-2) inhibitors;

Sodium (Na⁺) channel blockers (e:g., PN3, SNS, brain type III, SCN10A, and SCN3A);

NR1/NR2B NMDA receptor antagonists;

Voltage-gated Calcium (Ca⁺²) channel antagonists (e.g., CACNA1B, CACNA1A, and CACNA1E);

Alpha-2-adrenoceptor agonists; and

5-HT1A receptor antagonists.

Nonlimiting examples of preclinically validated analgesics include the following:

Tachykinin-1, -2, and -3 receptor antagonists;

Cannabinoid receptor antagonists;

MAP kinase enzyme cascade inhibitors (e.g., MEK inhibitors);

GluR5 KA receptor antagonists;

mGluR1 and mGluR5 receptor (Group I metabotrophic glutamate receptors) antagonists;

Muscarinic M₄ and M₁ receptor antagonists;

Prostaglandin PGE2 (P₁) receptor antagonists;

Opiate delta receptor agonists; and

Adenosine kinase inhibitors.

Opioid analgesics are generally defined to be those compounds that relieve pain and bind to opioid receptors. Opioid agonists and opioid agonist-antagonists are opioid analgesics and are addictive when administered to treat a mammal for pain. Opioid antagonists are nonaddictive. Nonlimiting examples of opioid analgesics include opiates, opiate derivatives, opioids, and their pharmaceutically acceptable salts. Specific nonlimiting examples of opioid agonist analgesics include alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, morphine, neperidine, oxycodone, phenomorphan, phenoperidine, piritradide, pholcodine, proheptazoine, properidine, propiran, racemoramide, thebacon, trimeperidine, and the pharmaceutically acceptable salts thereof. Specific nonlimiting examples of opioid agonist-antagonist analgesics include pentazocine, butorphanol, nalbuphine, buprenorphine, and the pharmaceutically acceptable salts thereof. Specific nonlimiting examples of opioid antagonist analgesics include naloxone, naltrexone, and the pharmaceutically acceptable salts thereof.

The phrase “neurokinin 1 receptor antagonist” means nontoxic compounds that block or interfere with binding to the NK₁ receptor subtype. Preferred NK₁ receptor antagonists that can be used herein are disclosed in U.S. Pat. No. 5,594,022. Among these, a more preferred NK₁ receptor antagonist is [2-(1H-indol-3-yl)-1-methyl-1-(1-phenyl-ethylcarbamoyl)-ethyl]-carbamic acid benzofuran-2-ylmethyl ester.

The expression “N-methyl-D-aspartate receptor” shall be understood to include all of the binding site subcategories associated with the NMDA receptor, e.g., the glycine-binding site, the phencyclidine (PCP)-binding site, etc., as well as the NMDA channel. The invention, herein contemplates the use of nontoxic substances that block or interfere with an NMDA receptor binding site. In one preferred embodiment NMDA receptor antagonists which can be used in the novel combinations are compounds that block or reduce the effects of NMDA at the NMDA subclass of neuronal glutamate receptors (nonlimiting examples include dextrorphan, dextromethorphan, and ketamine) or that block or interfere with the NMDA channel (e.g., a substance that blocks the magnesium or calcium channel). In another preferred embodiment, the NMDA receptor antagonist is one which is specific for a subtype of NMDA receptor, those containing the NR2B subunit which are expressed in the forebrain (nonlimiting examples include (1S,2S)-1-(4-hydroxyphenyl) 2-(4-hydroxy-4-phenylpiperidine)-1-propanol). Other NMDA receptor antagonists acting at other sites of an NMDA receptor include, but are not limited to 1H-indole-2-carboxylic acid, 4,6-dichloro-3-[3-oxo-3-(phenylamino)-1-propenyl]-, (E)- (GV-150526) (a compound in preclinical development by Glaxo Wellcome), 1-piperidineethanol, alpha.-(4-hydroxyphenyl)-.beta.-methyl-4-(phenylmethyl)- (ifenprodil), and ACEA's 1168.

The term “NSAID,” as used to describe other compounds useful in the novel combination herein, is intended to be a nonsteroidal anti-inflammatory compound. NSAIDs are categorized by virtue of their ability to inhibit cyclooxygenase. Cyclooxygenase 1 and cyclooxygenase 2 are the two major isoforms of cyclooxygenase and most standard NSAIDs are mixed inhibitors of the two isoforms. Most standard NSAIDs fall within one of the following five structural categories: (1) propionic acid derivatives, such as ibuprofen, naproxen, naprosyn, diclofenac, and ketoprofen; (2) acetic acid derivatives, such as tolmetin and sulindac; (3) fenamic acid derivatives, such as mefenamic acid and meclofenamic acid; (4) biphenylcarboxylic acid derivatives, such as diflunisal and flufenisal; and (5) oxicams, such as piroxim, peroxicam, sudoxicam, and isoxican. Other useful NSAIDs include aspirin, acetaminophen, indomethacin, and phenylbutazone.

Another class of NSAID has recently been described which selectively inhibits cyclooxygenase-2 (COX-2). These compounds reduce pain and inhibit the inflammatory response without damaging the gastric mucosa, a common toxicity observed with the mixed inhibitors.

(Z)-5-[[3,5-bis(1,1-dimethylethyl)-4-hydroxyphenyl]methylene]-2-imino-4-thiazolidinone methanesulfonate (1:1);

Celecoxib;

Meloxicam;

COX-189 (Novartis);

CS-502 (Sankyo);

Pyrano[3,4-b]indole-1-acetic acid, 1,8-diethyl-1,3,4,9-tetrahydro-(etodolac);

4-(4-Cyclohexyl-2-methyloxazol-5-yl)-2-fluorobenzenesulfonamide (JTE-522);

2-(3,5-Difluorphenyl)-3-(4-methylsulfonyl)phenyl)-2-cyclopenten-1-one (L-776967):

L-791456 (Merck & Co);

MK-663 (Merck & Co);

Methanesulfonamide;

N-(4-Nitro-2-phenoxyphenyl)- (nimesulide);

Propanamide;

N-((4-(5-Methyl-3-phenyl-4-isoxazolyl)phenyl)sulfonyl)- (parecoxib);

2(5H)-Furanone;

4-(4-(Methylsulfonyl)phenyl)-3-phenyl- (rofecoxib);

5(E)-(3,5-di-tert-Butyl-4-hydroxy)benzylidene-2-ethyl-1,2-isothiazolidine-1,1-dioxide (S-2474);

S-33516 (Servier);

Benzenesulfonamide, 4-(5-methyl-3-phenyl-4-isoxazolyl)- (valdecoxib);

CT 3 (Channel);

GR 253035 (Glaxo Wellcome);

N-[6-[(2,4-Difluorophenyl)thio]-2,3-dihydro-1-oxo-1H-inden-5-yl]methanesulfonamide (L 745337);

L 748731 (Merck & Co);

L 752860 (Merck & Co);.

(R)-1-[(4-bromophenyl)methyl]-5-methoxy-beta,2-dimethyl-1H-indole-3-butanoic acid (L 761066);

5-[4-(methylsulfonyl)phenyl]-6-phenylthiazolo[3,2-b][1,2,4]triazole (L 768277);

LAS 33826 (Almirall Prodesfama);

N-[5-[(4-fluorophenoxy)-2-thienyl]methansulfonamide (RWJ 63556);

SD 8381 (Searle);

and their pharmaceutically acceptable salts are examples of selective cyclooxygenase 2 inhibitors.

The phrase “antiepileptic compound” means a pharmaceutically active compound that treats disorders characterized by recurring attacks of motor, sensory, or psychic malfinction with or without unconsciousness or convulsive movement.

The phrase “antiepileptic compound having pain alleviating properties” means an antiepileptic compound as defined above which also alleviates pain in a mammal, including a human. Many antiepileptic compound having pain alleviating properties are known such as, for example, the structural analogs of gamma-aminobutyric acid (GABA) or glutamic acid. Examples are gabapentin, pregabalin, and 3-(1-aminomethyl-cyclohexylmethyl)-4H-[1,2,4]oxadiazol-5-one hydrochloride. Gabapentin has been shown to possess pain alleviating properties in animal models of inflammation and neuropathic pains, and in particular to block the maintenance of streptozotocin-induced static allodynia in the rat (Field et al., J. Pharmacol. Exp. Ther. 1997;282:1242-1246). Since determination of pain alleviating activity of an antiepileptic compound in an animal model of pain alleviation is well known in the pharmaceutical arts, it is a simple matter to test any antiepileptic compound for pain alleviating properties. Nonlimiting examples of antiepileptics having pain alleviating properties include gabapentin, pregabalin, 3-(1-aminomethyl-cyclohexylmethyl)-4H-[1,2,4]oxadiazol-5-one hydrochloride, carbamazepine, lamotrigine, phenytoin, fosphenytoin, and analogues thereof.

As noted above, the combinations of this invention may include any GABA analog. The preferred GABA analogs to be utilized in the method of this invention are cyclic amino acids of Formula I. These are described in U.S. Pat. No. 4,024,175 and its divisional U.S. Pat. No. 4,087,544, which are incorporated herein by reference. Another preferred method utilizes the GABA analogs of Formula II, and these are described in U.S. Pat. No. 5,563,175, which is incorporated herein by reference. Another preferred method utilizes the GABA analogs of Formula III, IIIC, IIIF, IIIG, and IIIH, and these are described in PCT International Application WO 99/31075, which is herein incorporated by reference. Another preferred method utilizes the GABA analogs of Formula IV.

Other preferred GABA analogs are described in PCT International Application No. WO 99/31074. Such GABA analogs are novel compounds of formula (1A) and (1B)

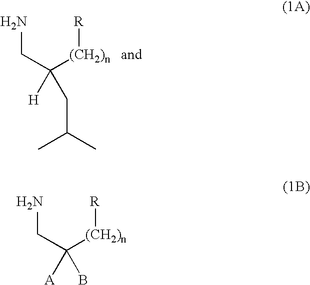

or a pharmaceutically acceptable salt thereof wherein:

n is an integer of from 0 to 2;

R is sulfonamide,

amide,

phosphonic acid,

heterocycle,

sulfonic acid, or

hydroxamic acid;

A is hydrogen or methyl; and

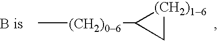

straight or branched alkyl of from 1 to 11 carbons, or —(CH₂)₁₋₄—Y—(CH₂)₀₋₄-phenyl wherein Y is —O—, —S—, —NR′₃ wherein:

R′₃ is alkyl of from 1 to 6 carbons, cycloalkyl of from 3 to 8 carbons, benzyl or phenyl wherein benzyl or phenyl can be unsubstituted or substituted with from 1 to 3 substituents each independently selected from alkyl, alkoxy, halogen, hydroxy, carboxy, carboalkoxy, trifluoromethyl, and nitro.

Other preferred GABA analogs are described in PCT International Application No. WO 99/31057. Such GABA analogs are novel compounds of formula (1D) and (1E)

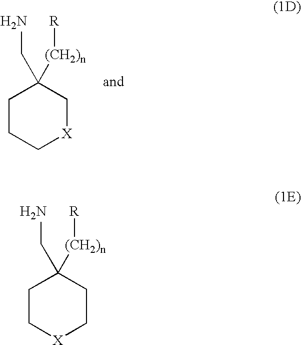

or a pharmaceutically acceptable salt thereof wherein:

n is an integer of from 0 to 2;

R is sulfonamide,

amide,

phosphonic acid,

heterocycle,

sulfonic acid, or

hydroxamic acid; and

X is —O—, —S—, —S(O)—, —S(O)₂—, or NR′₁ wherein R′₁ is hydrogen, straight or branched alkyl of from 1 to 6 carbons, benzyl, —C(O)R′₂ wherein R′₂ is straight or branched alkyl of 1 to 6 carbons, benzyl or phenyl or —CO₂R′₃ wherein R′₃ is straight or branched alkyl of from 1 to 6 carbons, or benzyl wherein the benzyl or phenyl groups can be unsubstituted or substituted by from 1 to 3 substituents selected from halogen, trifluoromethyl, and nitro.

Other preferred GABA analogs are described in PCT International Application No. WO 98/17627. Such GABA analogs are novel compounds of formula

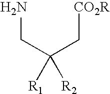

or a pharmaceutically acceptable salt thereof wherein:

R is hydrogen or lower alkyl;

R₁ is hydrogen or lower alkyl;

R₂is

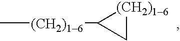

straight or branched alkyl, of from 7 to 11 carbon atoms, or —(CH₂)₍₁₋₄₎—X—(CH₂)₍₀₋₄₎-phenyl wherein

X is —O—, —S—, —NR₃— wherein

R₃ is alkyl of from 1 to 6 carbons, cycloalkyl of from 3 to 8 carbons, benzyl or phenyl;

wherein phenyl and benzyl can be unsubstituted or substituted with from 1 to 3 substituents each independently selected from alkyl, alkoxy, halogen, hydroxy, carboxy, carboalkoxy, trifluoromethyl, amino, and nitro.

Other preferred GABA analogs are described in PCT International Application No. WO 99/61424. Such GABA analogs are novel compounds of formulas (1), (2), (3), (4), (5), (6), (7), and (8)

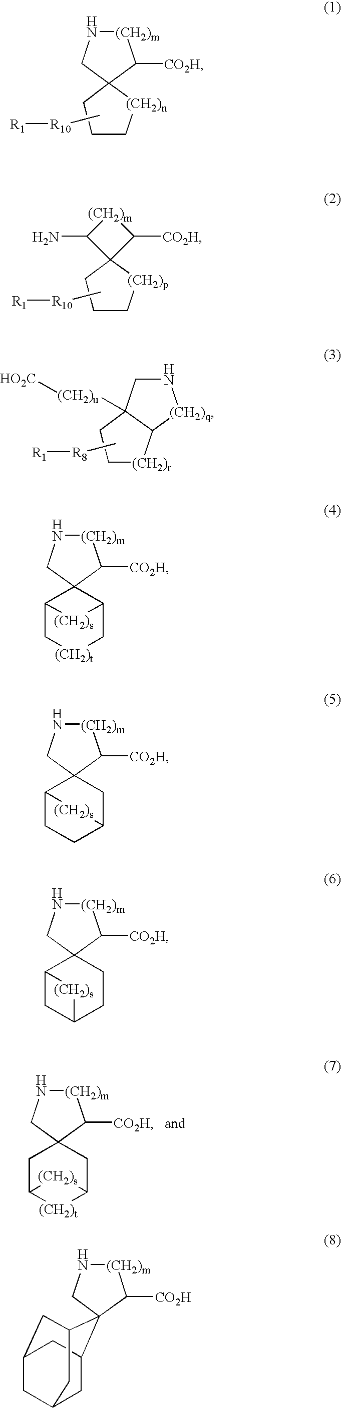

or a pharmaceutically acceptable salt thereof or a prodrug thereof wherein:

R₁ to R₁₀ are each independently selected from hydrogen or a straight or branched alkyl of from 1 to 6 carbons, benzyl, or phenyl;

m is an integer of from 0 to 3;

n is an integer of from 1 to 2;

o is an integer of from 0 to 3;

p is an integer of from 1 to 2;

q is an integer of from 0 to 2;

r is an integer of from 1 to 2;

s is an integer of from 1 to 3;

t is an integer of from 0 to 2; and

u is an integer of from 0 to 1.

International patent applications WO 99/31074, WO 99/31057, WO 98/17627, and WO 99/61424 are herein incorporated by reference.

Antiepileptic compounds having pain alleviating properties such as gabapentin and pregabalin are alpha-2-delta (gabapentin receptor) antagonists.

The term “pain alleviating properties” is generally defined herein to include the expressions “pain-suppressing,” “pain-reducing,” and “pain-inhibiting” as the invention is applicable to the alleviation of existing pain, as well as the suppression or inhibition of pain which would otherwise ensue from an imminent pain-causing event.

In addition to its pain alleviating properties, gabapehtin is extremely well-tolerated and has been demonstrated to be virtually free of drug interactions.

In the compounds of Formulas I through IV, the term “alkyl” means a straight or branched hydrocarbon radical having from 1 to 12 carbon atoms unless otherwise specified and includes, for example, methyl, ethyl, n-propyl, isopropyl, n-butyl, sec-butyl, isobutyl, tert-butyl, allyl, n-pentyl, n-hexyl, n-heptyl, n-octyl, n-nonyl, n-decyl, undecyl, and dodecyl.

The phrase “lower alkyl” means a straight or branched chain alkyl group of from 1 to 8 carbon atoms unless otherwise specified and includes, for example, methyl ethyl, isobutyl, pentyl, 3,3-dimethylhexyl, and 2-ethylpentyl.

The term “alkenyl” means a straight or branched hydrocarbon radical having from 2 to 12 carbon atoms unless otherwise specified and having at least one double bond in the carbon atom chain and includes, for example, 1-ethene, 1-propene, 2-methyl-1-propene, 1-butene, 2-butene, 1-pentene, 2-pentene, 2-methyl-1-butene, 3-methyl-1-butene, 3-methyl-2-butene, 1-hexene, 1-heptene, 1-octene, 1-nonene, 1-decene, 1-undecene, 1-dodecene, and the like.

The term “alkynyl” means a straight or branched hydrocarbon radical having from 2 to 12 carbon atoms unless otherwise specified and having at least one triple bond in the carbon atom chain and includes, for example, 1-ethyne, 1-propyne, 1-butyne, 3-methyl-1-butyne, 1-pentyne, 2-pentyne, 1-hexyne, and the like.

The term “cycloalkyl” means a saturated hydrocarbon ring which contains from 3 to 12 carbon atoms unless otherwise specified, for example, cyclopropyl, cyclobutyl, cyclopentyl, cyclohexyl, cycloheptyl, and adamantyl.

The phrase “lower cycloalkyl” means a saturated hydrocarbon ring which contains from 3 to 8 carbon atoms unless otherwise specified and includes, for example, cyclopropyl, cyclobutyl, cyclopentyl, cyclohexyl, cycloheptyl, and cyclooctyl.

“Halogen” is fluorine, chlorine, bromine, or iodine.

Some of the analgesics, antiepileptics having pain alleviating properties, and endothelin antagonists related to the invention, including compounds ofFormulas I through VI, are capable of further forming nontoxic pharmaceutically acceptable acid addition and/or base salts. All of these forms are within the scope of the present invention.

Pharmaceutically acceptable acid addition salts of the analgesics, antiepileptics having pain alleviating properties, and endothelin antagonists related to the invention, including compounds of Formulas I through VI, include salts derived from inorganic acids such as hydrochloric, nitric, phosphoric, sulfuric, hydrobromic, hydriodic, hydrofluoric, phosphorous, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al., “Phannaceutical Salts,” Journal of Pharmaceutical Science 1977;66:1-19).

The acid addition salts of said basic compounds are prepared by contacting the free base form with a sufficient amount ofthe desired acid to produce the salt in the conventional manner.

Pharmaceutically acceptable base addition salts are formed with metal cations, such as alkali and alkaline earth metal cations or armines such as organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example Berge S. M., Supra., 1977).

The base addition salts of said acidic compounds are prepared by contacting the free acid form with a sufficient amount of the desired base to produce the salt in the conventional manner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

Certain of the compounds of the present invention, possess one or more chiral centers and each center may exist in the R or S configuration. The present invention includes all enantiomeric and epimeric forms as well as the appropriate diastereomeric mixtures thereof.

### Materials and Methods

The method of Jarvis et al. is one method used to test the pain alleviating effects of the combinations of the instant invention and is hereby incorporated by reference (Jarvis M. F., Wessale J. L., Zhu C. Z., et al., Eur. J. Pharmacol. 2000;388:29-35).

A method for testing the novel combinations of the invention for pain alleviating activity in patients suffering from static or dynamic allodynia—forms of diabetic peripheral neuropathy—is described in Field M. J., Hughes J., Singh L., Pain 1999;80:391-398, which is hereby incorporated by reference.

Static and Dynamic Allodynia Methods:

1. Animals

Male Sprague-Dawley rats (175-200 g), obtained from Bantin and Kingman (Hull, UK), were housed in groups of six under a 12:12 hours/hours light-dark cycle (lights on at 07:00 hours) with food and water ad libitum. All experiments were carried out by an observer unaware of drug treatments.

2. Development of Diabetes in the Rat

Diabetes was induced by a single i.p. injection of streptozocin at a dose of 50 mg/kg. Control animals received a similar administration of isotonic saline.

3. Evaluation of Static Allodynia

Static allodynia was measured using Semmes-Weinstein von Frey hairs (Stoelting, Wood Date, Ill.). The animals were habituated to wire mesh bottom cages prior to the start of the experiment. Static allodynia was evaluated by application of von Frey hairs in ascending order of force (0.7, 1.2, 1.5, 2, 3.6, 5.5, 8.5, 11.8, 15.1, and 29 g) to the plantar surface of the rat's right hind paw. Each von Frey hair was applied to the paw for 6 seconds, or until a withdrawal response occurred. Once a withdrawal response was established, the paw was retested, starting with the next descending von Frey hair until no response occurred. The highest force of 29 g lifted the paw, as well as elicited a response, and thus represented the cut off point. The lowest amount of force required to elicit a response was recorded as PWT in grams.

4. Evaluation of Dynamic Allodynia

The animals are habituated to wire mesh bottom cages prior to the start of the experiment. Care is taken to perform this procedure in fully habituated rats that are not active to avoid recording general motor activity. Dynamic allodynia is assessed by lightly stroking the plantar suface of the rat's right hind paw with a cotton bud. Latency to paw withdrawal is noted. At least three measurements are taken at each time point. If no reaction is exhibited within 15 seconds, the procedure is terminated, and animals are assigned this withdrawal time. Thus 15 seconds effectively respresents no withdrawal. A withdrawal response is often accompanied by repeated flinching or licking of the paw. Animals are only selected for drug studies if they exhibit a withdrawal time of 8 seconds or less.

5. Development of Hyperglycemia and the Static Component (and Dynamic Component) of Streptozocin-Induced Mechanical Allodynia

Withdrawal thresholds to von Frey hairs were assessed on various days after the induction of diabetes. Animals with static allodynia were defined as those responding to 3.63 g or a lower strength von Frey hair. (Withdrawal latencies to a cotton bud stimulus is also assessed on various days after the induction of diabetes. The same group of rats may be used to assess both withdrawal latencies and withdrawal thresholds. Animals with dynamic allodynia are defined as animals responding to the cotton stimulus within 8 seconds of stroking.) Blood glucose levels were measured in the same animals after each day of testing, using a Glucotrend meter and test strips (Boehringer Mannhiem, Lewes, UK) to examine the development of hyperglycemia. Vehicle-treated controls were also examined for blood glucose levels on the same day.

6. Evaluation of the Effect of Compounds on Ssatic Allodynia (and Dynamic Allodynia)

The antiallodynic potential of a compound administered alone was examined in streptozocin-treated animals once dynamic and static allodynia had developed. Baseline paw withdrawal thresholds to von Frey hairs (or paw withdrawal latencies to a cotton bud) were determined on each test day before drug administration. After drug administration, animals were re-evaluated using von Frey hairs (or cotton bud) at 0.5, 1, 2, and up to 4 hours. Using this data, the time point postdose for peak activity of each individual compound were determined. The antiallodynic potential of a combination of the invention was then determined at the time point postdose that was expected to show peak activity for the combination.

7. Drugs

Streptozocin was purchased from Aldrich (UK). All compounds were dissolved in normal saline and administered orally. Drug administrations were made in a volume of 1 mL/kg.

8. Data Analysis

Data obtained from the static allodynia studies were analyzed using a one-way ANOVA followed by a Dunnett's t-test and an individual Mann-Whitney U-test. In each case the drug-treated groups were compared with the appropriate vehicle-treated group. (Data obtained from dynamic allodynia studies is also analyzed using these methods.)

9. Development of Streptozocin-Induced Static (and Dynamic) Allodynia

The development of streptozocin-induced static allodynia followed distinct time courses. Static allodynia could be detected from Day 4 post-streptozocin and was present in the majority of animals within 10 days, with animals demonstrating a PWT to the previously innocuous 3.63 g or lower force. (Dynamic allodynia follows a slower onset. It is not exhibited by all animals, with approximately 60% displaying a PWT of 8 seconds or less at 18 days post-streptocozin.) Vehicle-treated control animals exhibited a consistent PWT of 8.5 to 11.75 g to von Frey stimulus. (Vehicle-treated animals find the cotton bud stimulus innocuous throughout the dynamic allodynia studies.) Vehicle-treated animals had consistent blood glucose levels, similar to baseline values, throughout the experiment. Streptozocin-treated animals had elevated blood glucose values from the first day of testing, Day 4 post-streptozocin dose.

The static allodynia method of Field et al., was used in Tests 1 and 2, and Examples 1 through 3 below.

Test 1

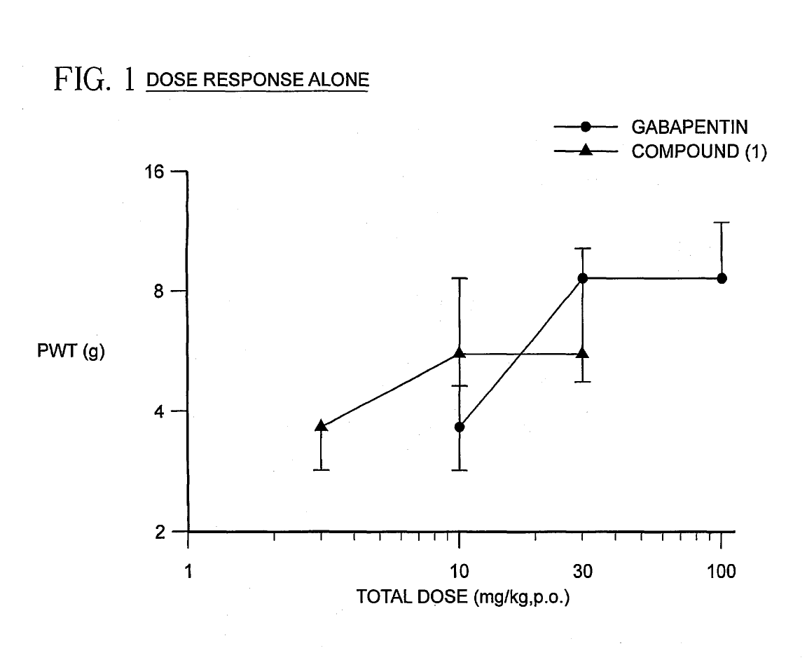

Gabapentin was administered orally alone at doses of 10, 30, or 100 mg/kg of rat body weight, and PWT was determined at 1 hour postdose, the time point of peak activity for this compound. The results are illustrated in FIG. 1 with the line

[symbol]

Test 2

The compound of formula (1) was administered orally alone at doses of 3, 10, or 30 mg/kg of rat body weight, and PWT was determined at 1 hour postdose, the time point of peak activity for this compound. The results are illustrated in FIG. 1 with the line

[symbol]

### EXAMPLE 1

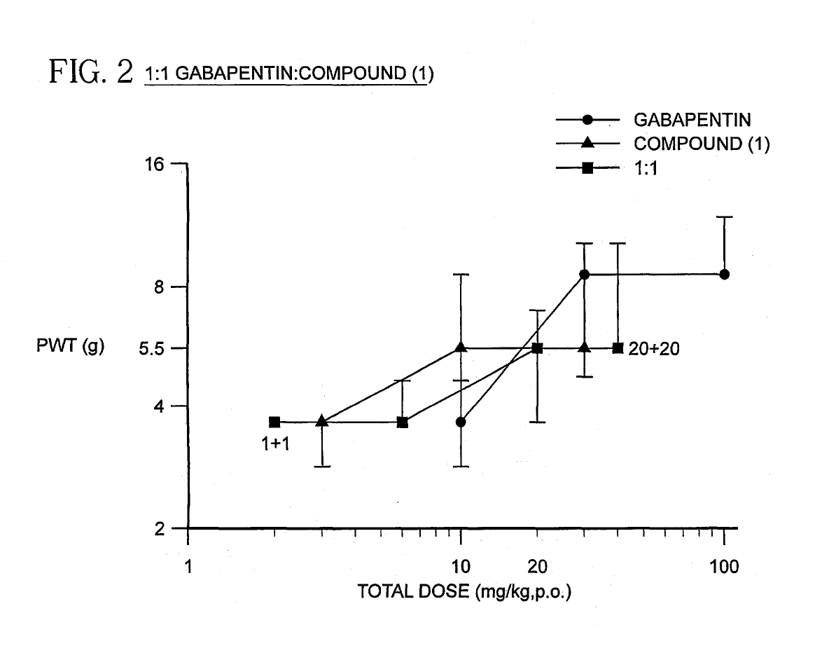

A combination of gabapentin and the compound of formula (1) at a 1:1 ratio, respectively, was administered orally at total doses (dose of gabapentin+dose of the compound of formula (1) of 2 mg/kg (1 mg/kg+1 mg/kg, respectively), 6 mg/kg (3 mg/kg+3 mg/kg, respectively), 20 mg/kg (10 mg/kg+10 mg/kg, respectively), or 40 mg/kg (20 mg/kg+20 mg/kg, respectively), and PWT was determined at 1 hour postdose. The results are illustrated in FIG. 2 with the line [symbol] . For comparison purposes, the results from Tests 1 and 2 are also illustrated in FIG. 2.

### EXAMPLE 2

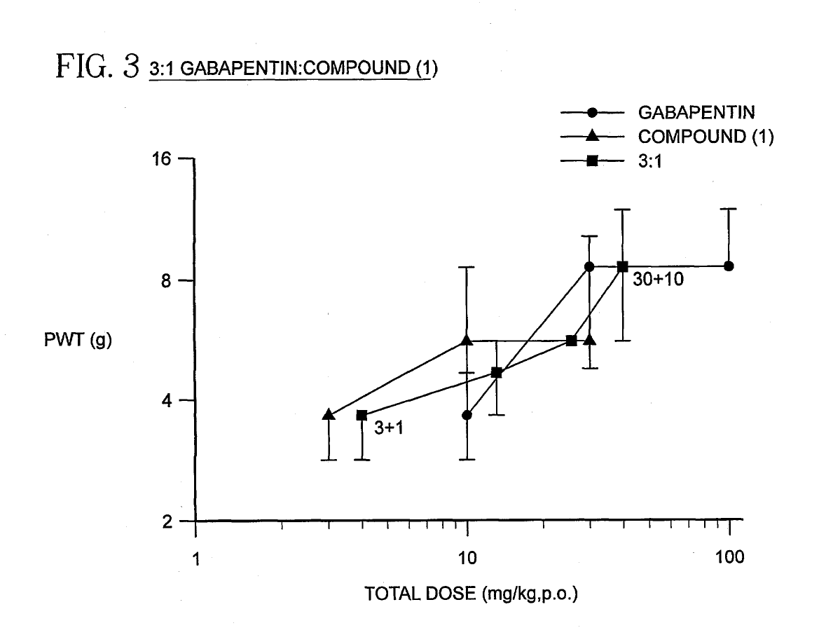

A combination of gabapentin and the compound of formula (1) at a 3:1 ratio, respectively, was administered orally at total doses (dose of gabapentin+dose of the compound of formula (1) of 4 mg/kg (3 mg/kg+1 mg/kg, respectively), 12 mg/kg (9 mg/kg+3 mg/kg, respectively), 28 mg/kg (21 mg/kg+7 mg/kg, respectively), or 40 mg/kg (30 mg/kg+10 mg/kg, respectively), and PWT was determined at 1 hour postdose. The results are illustrated in FIG. 3 with the line [symbol] . For comparison purposes, the results from Tests 1 and 2 are also illustrated in FIG. 3.

### EXAMPLE 3

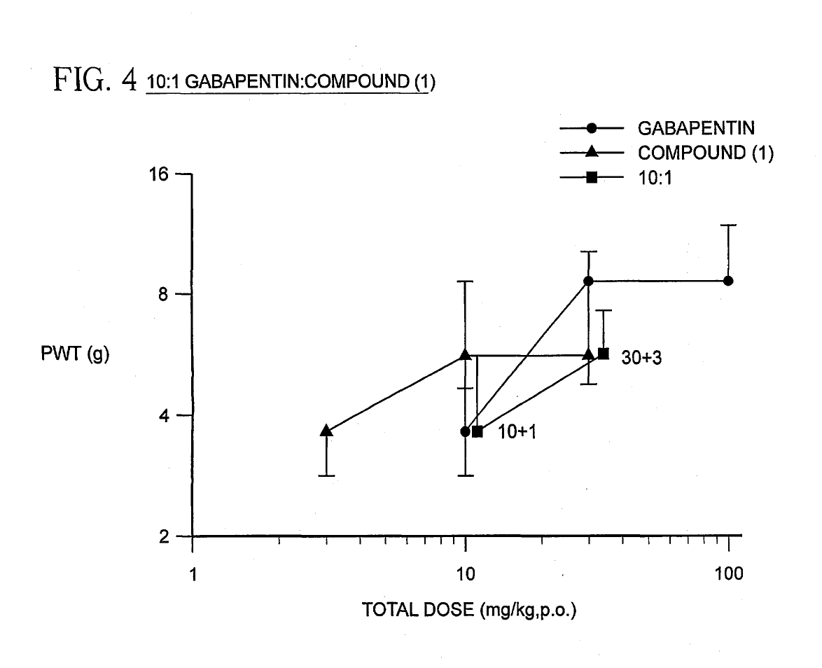

A combination of gabapentin and the compound of formula (1) at a 10:1 ratio, respectively, was administered orally at total doses (dose of gabapentin+dose of the compound of formula (1) of 11 mg/kg (10 mg/kg+1 mg/kg, respectively) or 33 mg/kg (30 mg/kg+3 mg/kg, respectively), and PWT was determined at 1 hour postdose. The results are illustrated in FIG. 4 with the line [symbol] . For comparison purposes, the results from Tests 1 and 2 are also illustrated in FIG. 4.

The results of Tests 1 and 2 and Examples 1 through 3 are illustrated in FIGS. 1 through 4 as described above. All data illustrated in the figures were determined at 1 hour postdose. In FIG. 1, a dose-response curve for gabapentin administered alone shows that at a dose of 10 mg/kg, gabapentin had little or no effect on static allodynia, while at doses of 30 and 100 mg/kg, gabapentin effected essentially a complete reversal of static allodynia. (As mentioned above, the presence of complete static allodynia is characterized by a PWT of ≦3.63 g, while the absence of static allodynia is characterized by a PWT of ≧8.5 g.) Also in FIG. 1 is a dose-response curve for the compound of formula (1) administered alone. This curve shows that at a dose of 3 mg/kg, the compound of formula (1) had little or no effect on static allodynia, while at doses of 10 and 30 mg/kg, the compound of formula (1) effected a partial (about 50%) reversal of static allodynia.

In FIG. 2, a dose-response curve for a combination of gabapentin and the compound of formula (1) administered at a ratio of 1:1 (weight:weight), respectively, shows that at a total dose of 2 or 6 mg/kg, the combination had little or no effect on static allodynia, while at total doses of 20 or 40 mg/kg, the combination effected partial reversal of static allodynia. Also in FIG. 2 for comparison purposes are the dose-response curves described for FIG. 1.

In FIG. 3, a dose-response curve for a combination of gabapentin and the compound of formula (1) administered at a ratio of 3:1 (weight:weight), respectively, shows that at a total dose of 4 mg/kg, the combination had little or no effect on static allodynia, while at total doses of 12 or 28 mg/kg, the combination effected partial reversal of static allodynia. Further at a total dose of 40 mg/kg, this combination effected a complete reversal of static allodynia. Also in FIG. 3, for comparison purposes, are the dose-response curves described for FIG. 1.

In FIG. 4, a dose-response curve for a combination of gabapentin and the compound of formula (1) administered at a ratio of 10:1 (weight:weight), respectively, shows that at a total dose of 11 mg/kg, the combination had little or no effect on static allodynia, while at a total dose of 33 mg/kg, the combination effected partial reversal of static allodynia. Also in FIG. 4, for comparison purposes, are the dose-response curves described for FIG. 1.

The results illustrated in FIGS. 2 through 4 show that the combinations of the present invention are effective at reversing static allodynia, and are thus useful for the treatment of pain.

Alternatively, a rat carrageenan-induced thermal hyperalgesia model may be used to determine pain alleviating effects as follows.

Animals

Male Sprague-Dawley rats (200-250 g, Sasco Laboratories) are used. Rats are group housed 5/cage on a 12-hour light:dark cycle with free access to food and water. Rats receive only one dose of a drug or drug combination. All drugs are administered orally by gavage.

Experimental Design

Dose-effect curves are first determined for (1) an endothelin antagonist by itself, (2) antiepileptic compound having pain alleviating properties by itself, and/or (3) an analgesic (e.g., naproxen) by itself. The ED₅₀ value and 95% confidence limits of each agent is determined, as is the time to peak effect. After determination of these values, dose effect curves are generated for the endothelin antagonist administered in a fixed dose ratio with the antiepileptic compound having pain alleviating properties and/or the analgesic; the drugs are administered so that their peak effects are coincident. ED₅₀ values and 95% confidence limits are then determined for the drugs in combination.

Measures of Antinociception

Carrageenan-Induced Thermal Hyperalgesia:

Rats are acclimated to a testing chamber whose glass floor is maintained at 25° C. Thirty minutes later, a high intensity beam of light is focused through the glass on the ventral surface of each hindpaw, and the latency to reflex withdrawal of the paw from the light beam is measured to the nearest 0.1 second. This latency is termed the paw flick latency (PFL). Two measurements of PFL spaced 20 minutes apart are made for each paw, and the second measurement is taken as the baseline response latency. After determination of baseline PFL, 100 μL of 2% lambda-carrageenan is injected in the plantar surface of one hindpaw and the animal is returned to the testing chamber. Two hours later, when thermal hyperalgesia is maximal and stable, either vehicle, (1) the endothelin antagonist by itself, (2) the antiepileptic compound having pain alleviating properties by itself, (3) the analgesic by itself, or (4) a combination of the invention is administered by gavage. Response latencies for the ipsilateral and contralateral hindpaws are then re-determined 15, 30, 45, 60, 90, and 120 minutes later. Data for further analysis are taken 120 minutes after oral dosing.

Statistical Analysis

Data are expressed as the mean±SEM. Two-way analyses of variance for repeated measures is used to compare the effects of drug to that of vehicle. Dose-effect lines for each compound administered by itself are constructed using individual data and fitted with least squares linear regression analysis to determine ED₅₀ values and 95% confidence limits. A similar analysis is conducted for the compounds in combination using the total dose administered. Since parallel dose-effect lines are obtained for each compound administered by itself, and the combination of the invention, then a parallel line assay is conducted as described by Tallarida (Tallarida, 1992; Tallarida et al; 1989). This analysis compares the position of the experimentally-derived dose-effect line for the combination to the position of the theoretical dose-additive line. A significant shift to the left or the right of the theoretical dose-additive line indicates that the compounds interact in a supra-additive (synergistic) or an infra-additive manner (antagonistic), respectively.

The amount of the active ingredients in the combinations will vary depending on the mammal to which the combinations are administered, the type of pain to be treated, other active ingredients present, etc. Generally, the amount of the endothelin receptor antagonist(s) and the other active compound(s) for a given composition and dosage form can be readily determined employing routine procedures.

The combinations of the present invention can be prepared and administered in a wide variety of oral and parenteral dosage forms. Thus, the combinations of the present invention can be administered by injection, that is, intravenously, intramuscularly, intracutaneously, subcutaneously, intraduodenally, or intraperitoneally. Also, the combinations of the present invention can be administered by inhalation, for example, intranasally. Additionally, the combinations of the present invention can be administered transdermally. It will be obvious to those skilled in the art that the following dosage forms may comprise as the active components, a compound of Formula V or Formula VI, or a corresponding pharmaceutically acceptable salt thereof and from one to three compounds selected from the group consisting of antiepileptic compounds having pain alleviating properties and analgesics, or pharmaceutically acceptable salts thereof.

For preparing pharmaceutical compositions from the combinations of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component(s).

In tablets, the active component(s) is mixed with the carrier having the necessary binding properties in suitable proportions and compacted in the shape and size desired.

The powders and tablets preferably contain from 5% to about 70% of the active combinations. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound(s) with encapsulating material as a carrier providing a capsule in which the active component(s), with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component(s) is dispersed homogeneously therein as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component(s) in water and adding suitable colorants, flavors, stabilizing, and thickening agents as desired.

Aqueous suspensions suitable for oral use can be made by dispersing the finely divided active component(s) in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of each active component in a unit dose preparation may be varied or adjusted from 1 to 1000 mg, preferably 10 to 100 mg according to the particular application and the potency of each active component. The composition can, if desired, also contain other compatible therapeutic agents.

Co-administration of an endothelin receptor antagonist and from one to three compounds selected from the group consisting of antiepileptic compounds having pain alleviating properties and analgesics may comprise simultaneous or sequential administration of each pharmaceutically active compound that has been independently formulated by itself. Alternatively, co-administration may comprise administration of an endothelin receptor antagonist and from one to three compounds selected from the group consisting of antiepileptic compounds having pain alleviating properties and analgesics wherein the pharmaceutically active compounds of the combination have been combined in a single formulation. Further, co-administration may comprise a combination of these two approaches wherein certain pharmaceutically active compounds are formulated together and others are independently formulated by themselves, and the formulations are administered simultaneously or sequentially.

In therapeutic use as agents for the treatment of pain, the analgesics, antiepileptic compounds having pain alleviating properties, and endothelin antagonist compounds utilized in the pharmaceutical method of this invention can each be administered at the initial dosage of about 1 to about 100 mg/kg daily. A daily dose range of about 1 to about 75 mg/kg is preferred. Further, a concentration range of the antiepileptic and/or analgesic between 1% and 99% and a complementary concentration range of endothelin antagonist compound between 99% and 1% of the 100% total of the combination of active ingredients is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages that are less than the optimum dose of the compound. Thereafter, the dosage is increased by small increments until the optimum effect under the circumstance is reached. For convenience, the total daily dosage may be divided and administered in portions during the day if desired.

An example of an oral formulation follows.

### EXAMPLE 4

Tablet Formulation:

| Ingredient | Amount (mg) |
|---|---|
| 4-(7-ethyl-1,3-benzodioxol-5-yl)-2-[2-(trifluoromethyl)- | 25 |
| phenyl]-2H-1,2-dihydro-1,2-benzothiazine-3-carboxylic | |
| acid,1,1-dioxide, potassium salt | |
| Gabapentin | 25 |
| Lactose | 50 |
| Cornstarch (for mix) | 10 |
| Cornstarch (paste) | 10 |
| Magnesium stearate (1%) | 5 |
| Total | 125 |

The 1,2-benzothiazine-3-carboxylic acid, 1,1-dioxide, potassium salt, gabapentin, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pain.

Having described the present invention above, various embodiments of the invention are hereforth claimed.

What is claimed is:

1. A combination effective for alleviating pain comprising a pain alleviating effective amount of an endothelin antagonist or a pharmaceutically acceptable salt thereof and from one to three compounds independently selected from the group consisting of antiepileptic compounds having pain alleviating properties and analgesics, and pharmaceutically acceptable salts thereof.

2. The combination of claim I wherein the antiepileptic compound having pain alleviating properties is a compound of Formula I 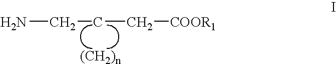
wherein R₁ is hydrogen or a lower alkyl; n is an integer of from 4 to 6; and
the cyclic ring is optionally substituted by lower alkyl or lower cycloalkyl, and the pharmaceutically acceptable salts thereof.

3. The combination of claim 1 wherein the antiepileptic compound having pain alleviating properties is gabapentin.

4. The combination of claim 1 wherein the antiepileptic compound having pain alleviating properties is a compound of Formula II 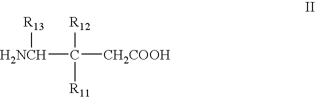
wherein R₁₁ is a straight or branched alkyl of from 1 to 6 carbon atoms, phenyl, or cycloalkyl having from 3 to 6 carbon atoms; R₁₂ is hydrogen or methyl; and R₁₃ is hydrogen, methyl, or carboxyl; or an individual diastereomeric or enantiomeric isomer thereof; or a pharmaceutically acceptable salt thereof.

5. The combination according to claim 1 wherein the antiepileptic compound having pain alleviating properties is a compound of Formula 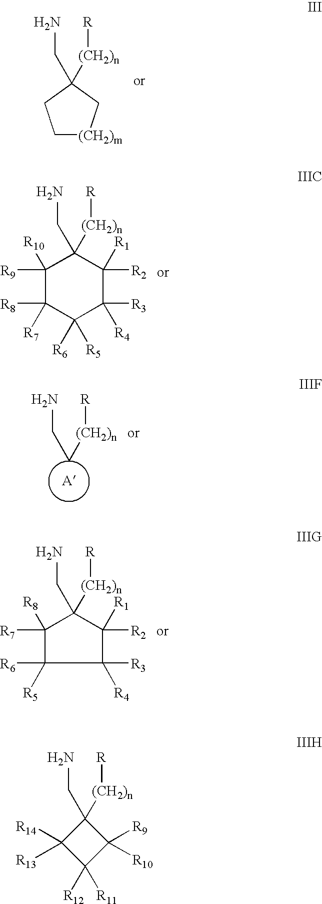
or a pharmaceutically acceptable salt thereof wherein:
n is an integer of from 0 to 2;
m is an integer of from 0 to 3;
R is sulfonamide,
amide,
phosphonic acid,
heterocycle,
sulfonic acid, or
hydroxamic acid;
R₁ to R₁₄ are each independently selected from hydrogen or straight or branched alkyl of from 1 to 6 carbons, unsubstituted or substituted benzyl or phenyl which substituents are selected from halogen, alkyl, alkoxy, hydroxy, carboxy, carboalkoxy, trifluoromethyl, and nitro;
A′ is a bridged ring selected from 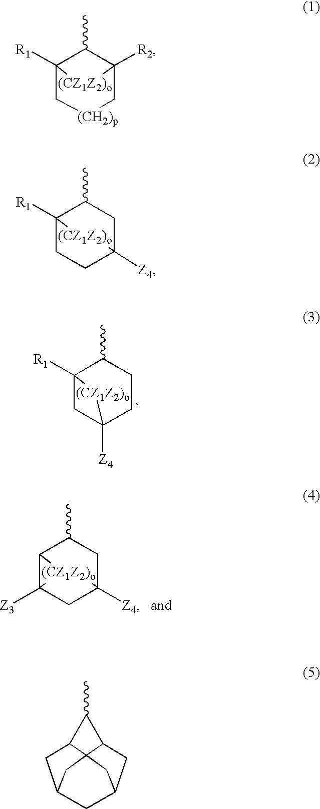
wherein [symbol]
is the point of attachment;
Z₁ to Z₄ are each independently selected from hydrogen and methyl;
o is an integer of from 1 to 4; and
p is an integer of from 0 to 2 with the proviso that in formula (1), R is not —SO₃H when m is 2 and n is 1.

6. The combination of claim 1 wherein the antiepileptic compound having pain alleviating properties is gabapentin.

7. The combination of claim 1 wherein the antiepileptic compound having pain alleviating properties is pregabalin.

8. The combination of claim 1 wherein the antiepileptic compound having pain alleviating properties is 3-(1-aininomethyl-cyclohexylmethyl)-4H-[1,2,4]oxadiazol-5-one hydrochloride.

9. The combination of claim 1 wherein the endothelin antagonist is selected from a compound of Formula V 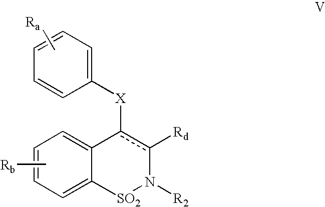
or a pharmaceutically acceptable acid addition or base salt thereof wherein:
R₂ is H, 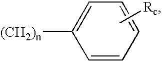
alkyl of from 1 to 7 carbons, (CH₂)ₙ-cycloalkyl of from 3 to 8 carbons;
Rₐ and R_{c} are each 1 to 5 substituents and R_{b} is from 1 to 4 substituents independently selected from:
hydrogen,
alkyl of from 1 to 7 carbons,
alkenyl of from 2 to 7 carbons,
alkynyl of from 2 to 7 carbons,
cycloalkyl of from 3 to 8 carbons, phenyl,
C(O)-phenyl,
methylenedioxy,
ethylenedioxy,
OR,
NRR₁,
SR₁,
NO₂,
N₃,
COR,
CO₂R,
Cl,
Br,
F,
I,
CONRR₁,
SO₂NRR₁,
SO₂R,
CN,
CF₃,
CF₂CF₃,
CHO,
OCOR,
B(OH)₂,
NH(CH₂)ₚCO₂R,
S(CH₂)ₚCO₂R,
O(CH₂)ₚCO₂R,
O(CH₂)ₚOR,
NH(CH₂)ₚOR,
S(CH₂)ₚOR, or
wherein R and R₁ are each independently selected from
hydrogen,
alkyl of from 1 to 6 carbon atoms,
alkenyl of from 2 to 7 carbon atoms,
alkynyl of from 2 to 7 carbon atoms,
cycloalkyl of from 3 to 8 carbon atoms,
phenyl or benzyl wherein the phenyl or benzyl ring is substituted by one or more hydrogen, methoxy, and methylenedioxy substituents;
R_{d} is H, CO₂R, SO₃R, PO₃R, B(OH)₂, CONRR₁, SO₂NRR₁, C(O)NHSO₂R₁, 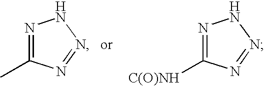
n is an integer of from 0 to 2;
p is an integer of from 1 to 4;
-- indicates a single or double bond; and
X is (CH₂)ₙ, O, NR, or S(O)ₙ.

10. The combination of claim 1 wherein the endothelin antagonist is selected from a compound of Formula VI 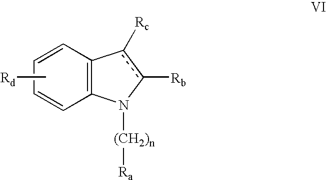
wherein
--- denotes an optional bond;
n is 0 to 4;
Rₐ is hydrogen, alkyl of 1-4 carbon atoms or cycloalkyl, phenyl or naphthyl, in which the phenyl or naphthyl group is substituted by methylenedioxy and further unsubstituted or substituted by one or more substituents selected from the group consisting of halogen, alkyl of 1-6 carbon atoms, OR, NRR¹, SR, NO₂, N₃, COR, CO₂R, CONRR¹, SO₂NRR¹, SO₂R, CN, CF₃, CF₂CF₃, CHO, OCOCH₃, B(OH)₂, phenyl, NH(CH₂)ₘCO₂R, S(CH₂)ₘCO₂R, O(CH₂)ₘCO₂R, O(CH₂)ₘOR, NH(CH₂)ₘOR and S(CH₂)ₘOR, in which m is 1, 2 or 3, and R and R¹ are each independently hydrogen, alkyl of 1-4 carbon atoms, phenyl or benzyl;
R_{b} is hydrogen, CO₂R², 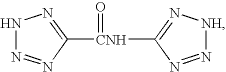
SO₃R, PO₃H, B(OH)₂, CONR¹R², SO₂NR¹R², or 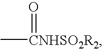
in which R¹ is as defined above and R² is hydrogen, alkyl of 1-6 carbon atoms, CF₃, —CF₂CF₃, phenyl or benzyl in which phenyl or the phenyl portion of the benzyl group is unsubstituted or substituted by one or more substituents as defined above;
R_{c} is S(O)ₚ-phenyl, in which p is 0, 1 or 2, and phenyl is unsubstituted or substituted by one or more substituents selected from the group consisting of halogen, OR, NRR¹, SR, NO₂, N₃, COR, CO₂R, CONRR¹, SO₂NRR¹, SO₂R, CN, CF₃, CF₂CF₃, CHO, OCOCH₃, B(OH)₂, methylenedioxy, NH(CH₂)ₘCO₂R, S(CH₂)ₘCO₂R, O(CH₂)ₘCO₂R, O(CH₂)ₘOR, NH(CH₂)ₘOR and S(CH₂)ₘOR, in which m, R and R¹ are as defined above, and
R_{d} is one to four independent substituents selected from hydrogen, alkyl of 1-7 carbon atoms, alkenyl of 2-7 carbon atoms, alkynyl of 2-7 carbon atoms, cycloalkyl, phenyl, C(O)-phenyl, X(CH₂)ₙ-phenyl, X—(CH₂)ₙ-naphthyl, in which X is 0, NH or S(O)ₚ, methylenedioxy, OR, NRR¹, SR, NO₂, N₃, COR, CO₂R, CONRR¹, SO₂NRR¹, SO₂R, CN, CF₃, CF₂CF₃, CHO, OCOCH₃, B(OH)₂, phenyl, NH(CH₂)ₘCO₂R, S(CH₂)ₘCO₂R, O(CH₂)ₘCO₂R, O(CH₂)ₘOR, NH(CH₂)ₘOR, S(CH₂)ₘOR, in which m is 1, 2 or 3 and R and R¹ are each independently hydrogen, alkyl of 1-4 carbon atoms, phenyl or benzyl and where n and p are as defined above and phenyl is unsubstituted or substituted as defined above, or a pharmaceutically acceptable acid addition or base salt thereof.

11. The combination of claim 1 wherein the endothelin antagonist is selected from:
4-Benzo[1,3]dioxol-5-yl-2-methyl-1,1-dioxo-1,2-dihydro-1λ⁶-benzo[e][1,2]thiazine-3-carboxylic acid;
4-Benzo[1,3]dioxol-5-yl-2-benzo[1,3]dioxol-5-ylmethyl-1,1-dioxo-1,2-dihydro-1λ⁶-benzo[e][1,2]thiazine-3-carboxylic acid;
4-Benzo[1,3]dioxol-5-yl-2-benzyl-1,1-dioxo-1,2-dihydro-1λ⁶-benzo[e][1,2]thiazine-3-carboxylic acid;
4-Benzo[1,3]dioxol-5-yl-2-(4-methoxy-benzyl)-1,1-dioxo-1,2-dihydro-1λ⁶-benzo[e][1,2]thiazine-3-carboxylic acid;
4-Benzo[1,3]dioxol-5-yl-1,1-dioxo-2-(3,4,5-trimethoxy-benzyl)-1,2-dihydro-1λ⁶-benzo[e][1,2]thiazine-3-carboxylic acid;
4-Benzo[1,3]dioxol-5-yl-2-(2-carboxymethoxy-4-methoxy-benzyl)-1,1-dioxo-1,2-dihydro-1λ⁶-benzo[e][1,2]thiazine-3-carboxylic acid;
4-Benzo[1,3]dioxol-5-yl-2-(6-chloro-benzo[1,3]dioxol-5-ylmethyl)-1,1-dioxo-1,2-dihydro-1λ⁶-benzo[e][1,2]thiazine-3-carboxylic acid;
4-Benzo[1,3]dioxol-5-yl-2-(7-methoxy-benzo[1,3]dioxol-5-ylmethyl)-1,1-dioxo-1,2-dihydro-1λ⁶-benzo[e][1,2]thiazine-3-carboxylic acid;
2-Benzo[1,3]dioxol-5-ylmethyl-4-(3,4-dimethoxy-phenyl)-1,1-dioxo-1,2-dihydro-1λ⁶-benzo[e][1,2]thiazine-3-carboxylic acid;
2-Benzo[1,3]dioxol-5-ylmethyl-1,1-dioxo-4-(3,4,5-trimethoxy-phenyl)-1,2-dihydro-1λ⁶-benzo[e][1,2]thiazine-3-carboxylic acid;
N-(4-Benzo[1,3]dioxol-5-yl-2-benzo[1,3]dioxol-5-ylmethyl-1,1-dioxo-1,2-dihydro-1λ⁶-benzo[e][1,2]thiazine-3-carbonyl)-benzenesulfonamide;
2-Benzo[1,3]dioxol-5-ylmethyl-4-(3-methoxy-phenyl)-1,1-dioxo-1,2-dihydro-1λ⁶-benzo[e][1,2]thiazine-3-carboxylic acid;.
4-Benzo[1,3]dioxol-5-yl-2-benzo[1,3]dioxol-5-ylmethyl-6,7-dimethoxy-1,1-dioxo-1,2-dihydro-1λ⁶-benzo[e][1,2]thiazine-3-carboxylic acid;
4-Benzo[1,3]dioxol-5-yl-2-benzo[1,3]dioxol-5-ylmethyl-6-methoxy-1,1-dioxo-1,2-dihydro-1λ⁶-benzo[e][1,2]thiazine-3-carboxylic acid;
8-Benzo[1,3]dioxol-5-yl-6-benzo[1,3]dioxol-5-ylmethyl-5,5-dioxo-5,6-dihydro-1,3-dioxa-5λ⁶-thia-6-aza-cyclopenta[b]naphthalene-7-carboxylic acid;
4-(Benzo[1,3]dioxol-5-ylsulfanyl)-2-methyl-1,1-dioxo-1,2-dihydro-1λ⁶-benzo[e][1,2]thiazine-3-carboxylic acid;
2-Benzo[1,3]dioxol-5-ylmethyl-4-(benzo[1,3]dioxol-5-ylsulfanyl)-1,1-dioxo-1,2-dihydro-1λ⁶-benzo[e][1,2]thiazine-3-carboxylic acid;
4-(Benzo[1,3]dioxol-5-ylsulfanyl)-2-benzyl-1,1-dioxo-1,2-dihydro-1λ⁶-benzo[e][1,2]thiazine-3-carboxylic acid;
4-(Benzo[1,3]dioxol-5-ylsulfanyl)-2-(4-methoxy-benzyl)-1,1-dioxo-1,2-dihydro-1λ⁶-benzo[e][1,2]tbiazine-3-carboxylic acid;
4-(Benzo[1,3]dioxol-5-ylsulfanyl)-1,1-dioxo-2-(3,4,5-trimethoxy-benzyl)-1,2-dihydro-1λ⁶-benzo[e][1,2]thiazine-3-carboxylic acid;
4-(Benzo[1,3]dioxol-5-ylsulfanyl)-2-(carboxymethoxy-4-methoxy-benzyl)-1,1-dioxo-1,2-dihydro-1λ⁶-benzo[e][1,2]thiazine-3-carboxylic acid;
4-(Benzo[1,3]dioxol-5-ylsulfanyl)-2-(6-chloro-benzo[1,3]dioxol-5-ylmethyl)-1,1-dioxo-1,2-dihydro-1λ⁶-benzo[e][1,2]thiazine-3-carboxylic acid;
4-(Benzo[1,3]dioxol-5-ylsulfanyl)-2-(7-methoxy-benzo[1,3]dioxol-5-ylmethyl)-1,1-dioxo-1,2-dihydro-1λ⁶-benzo[e][1,2]thiazine-3-carboxylic acid;
2-Benzo[1,3]dioxol-5-ylmethyl-4-(3,4-dimethoxy-phenylsulfanyl)-1,1-dioxo-1,2-dihydro-1λ⁶-benzo[e][1,2]thiazine-3-carboxylic acid;
2-Benzo[1,3]dioxol-5-ylmethyl-1,1-dioxo-4-(3,4,5-trimethoxy-phenylsulfanyl)-1,2-dihydro-1λ⁶-benzo[e][1,2]thiazine-3-carboxylic acid;
N-(4-Benzo[1,3]dioxol-5-ylsulfanyl-2-benzo[1,3]dioxol-5-ylmethyl-1,1-dioxo-1,2-dihydro-1λ⁶-benzo[e][1,2]thiazine-3-carbonyl)-benzenesulfonamide;
2-Benzo[1,3]dioxol-5-ylmethyl-4-(3-methoxy-phenylsulfanyl)-1,1-dioxo-1,2-dihydro-1λ⁶-benzo[e][1,2]thiazine-3-carboxylic acid;
2-Benzo[1,3]dioxol-5-ylmethyl-4-(benzo[1,3]dioxol-5-ylsulfanyl)-6,7-dimethoxy-1,1-dioxo-1,2-dihydro-1λ⁶-benzo[e][1,2]thiazine-3-carboxylic acid;
2-Benzo[1,3]dioxol-5-ylmethyl-4-(benzo[1,3]dioxol-5-ylsulfanyl)-6-methoxy-1,1-dioxo-1,2-dihydro-1λ⁶-benzo[e][1,2]thiazine-3-carboxylic acid;
6-Benzo[1,3[dioxol-5-ylmethyl-8-(benzo[1,3]dioxol-5-ylsulfanyl)-5,5-dioxo-5,6-dihydro-1,3-dioxa-5λ⁶-thia-6-aza-cyclopenta[b]-naphthalene-7-carboxylic acid;
4-(Benzo[1,3]dioxol-5-ylsulfanyl)-2-isobutyl-1,1-dioxo-1,2-dihydro-1λ⁶-benzo[e][1,2]thiazine-3-carboxylic acid;
2-Benzo[1,3]dioxol-5-ylmethyl-4-(benzo[1,3]dioxol-5-ylsulfanyl)-7-methoxy-1,1-dioxo-1,2-dihydro-1λ⁶-benzo[e][1,2]thiazine-3-carboxylic acid;
2-Benzo[1,3]dioxol-5-ylmethyl-4-(2,3-dihydro-benzo[1,4]dioxin-6-ylsulfanyl)-1,1-dioxo-1,2-dihydro-1λ⁶-benzo[e][1,2]thiazine-3-carboxylic acid;
4-(Benzo[1,3]dioxol-5-ylsulfanyl)-2-(2,3-dihydro-benzo[1,4]-dioxin-6-ylmethyl)-1,1-dioxo-1,2-dihydro-1λ⁶-benzo[e][1,2]thiazine-3-carboxylic acid;
4-(Benzo[1,3]dioxol-5-ylsulfanyl)-2-cyclohexylmethyl-1,1-dioxo-1,2-dihydro-1λ⁶-benzo[e][1,2]thiazine-3-carboxylic acid;
2-Benzo[1,3]dioxol-5-yl-4-(benzo[1,3]dioxol-5-ylsulfanyl)-1,1-dioxo-1,2-dihydro-1λ⁶-benzo[e][1,2]thiazine-3-carboxylic acid;
2-Benzo[1,3]dioxol-5-yl-4-(benzo[1,3]dioxol-5-ylsulfanyl)-6,7-dimethoxy-1,1-dioxo-1,2-dihydro-1λ⁶-benzo[e][1,2]thiazine-3-carboxylic acid;
2,4-Bis-benzo[1,3]dioxol-5-yl-1,1-dioxo-1,2-dihydro-1λ⁶-benzo[e][1,2]thiazine-3-carboxylic acid;
2,4-Bis-benzo[1,3]dioxol-5-yl-6,7-dimethoxy-1,1-dioxo-1,2-dihydro-1λ⁶-benzo[e][1,2]thiazine-3-carboxylic acid;
4-Benzo[1,3]dioxol-5-yl-2-(2-chloro-benzyl)-1,1-dioxo-1,2-dihydro-1λ⁶-benzo[e][1,2]thiazine-3-carboxylic acid;
2-Benzo[1,3]dioxol-5-ylmethyl-4-(4-chloro-2,6-dimethoxy-phenyl)-1,1-dioxo-1,2-dihydro-1λ⁶-benzo[e][1,2]thiazine-3-carboxylic acid;
4-(Benzo[1,3]dioxol-5-ylsulfanyl)-2-(2-chloro-benzyl)-1,1-dioxo-1,2-dihydro-1λ⁶-benzo[e][1,2]thiazine-3-carboxylic acid;
2-Benzo[1,3]dioxol-5-ylmethyl-4-(4-chloro-2,6-dimethoxy-phenylsulfanyl)-1,1-dioxo-1,2-dihydro-1λ⁶-benzo[e][1,2]thiazine-3-carboxylic acid;
4-(Benzo[1,3]dioxol-5-yl)-2-isobutyl-1,1-dioxo-1,2-dihydro-1λ⁶-benzo[e][1,2]thiazine-3-carboxylic acid;
2-Benzo[1,3]dioxol-5-ylmethyl-4-(benzo[1,3]dioxol-5-yl)-7-methoxy-1,1-dioxo-1,2-dihydro-1λ⁶-benzo[e][1,2]thiazine-3-carboxylic acid;
2-Benzo[1,3]dioxol-5-ylmethyl-4-(2,3-dihydro-benzo[1,4]dioxin-6-yl)-1,1-dioxo-1,2-dihydro-1λ⁶-benzo[e][1,2]thiazine-3-carboxylic acid;
4-(Benzo[1,3]dioxol-5-yl)-2-(2,3-dihydro-benzo[1,4]dioxin-6-ylmethyl)-1,1-dioxo-1,2-dihydro-1λ⁶-benzo[e][1,2]thiazine-3-carboxylic acid;
4-(Benzo[1,3]dioxol-5-yl)-2-cyclohexylmethyl-1,1-dioxo-1,2-dihydro-1λ⁶-benzo[e][1,2]thiazine-3-carboxylic acid;
1-Benzo[1,3]dioxol-5-yl-3-phenylsulfanyl-1H-indole-2-carboxylic acid;
1-Benzo[1,3]dioxol-5-ylmethyl-5,6-dimethoxy-3-(3-methoxy-phenylsulfanyl)-1H-indole-2-carboxylic acid;
1-Benzo[1,3]dioxol-5-ylmethyl-3-(3-methoxy-phenylsulfanyl)-1H-indole-2-carboxylic acid;
1-Benzyl-3-(3-methoxy-phenylsulfanyl)-1H-indole-2-carboxylic acid;
1-Benzo[1,3]dioxol-5-ylmethyl-3-(benzo[1,3]dioxol-5-ylsulfanyl)-1H-indole-2-carboxylic acid;
5-Benzo[1,3]dioxol-5-ylmethyl-7-(3-methoxy-phenylsulfanyl)-5H-[1,3]dioxolo[4,5-f]indole-6-carboxylic acid;
5-(7-Methoxy-benzo[1,3]dioxol-5-ylmethyl)-7-(3-methoxy-phenylsulfanyl)-5H-[1,3]dioxolo[4,5-f]indole-6-carboxylic acid;
5-Benzo[1,3]dioxol-5-ylmethyl-7-(3,4-dimethoxy-phenylsulfanyl)-5H-[1,3]dioxolo[4,5-f]indole-6-carboxylic acid;
7-(3,4-Dimethoxy-phenylsulfanyl)-5-(7-methoxy-benzo[1,3]-dioxol-5-ylmethyl)-5H-[1,3]dioxolo[4,5-f]indole-6-carboxylic acid;
1-Benzo[1,3]dioxol-5-ylmethyl-3-(3-methoxy-phenylsulfanyl)-6-propoxy-1H-indole-2-carboxylic acid;
5,6-Dimethoxy-1-(⁷-methoxy-benzo[1,3]dioxol-5-ylmethyl)-3-(3-methoxy-phenylsulfanyl)-1H-indole-2-carboxylic acid;
5,6-Dimethoxy-1-(4-methoxy-benzyl)-3-(3-methoxy-phenylsulfanyl)-1H-indole-2-carboxylic acid;
1-Benzo[1,3]dioxol-5-ylmethyl-5-benzyloxy-6-methoxy-3-(3-methoxy-phenylsulfanyl)-1H-indole-2-carboxylic acid;
1-Benzo[1,3]dioxol-5-ylmethyl-5,6-dimethoxy-3-(3,4,5-trimethoxy-phenylsulfanyl)-1H-indole-2-carboxylic acid;
1-Benzo[1,3]dioxol-5-ylmethyl-3-(benzo[1,3]dioxol-5-ylsulfanyl)-6-benzyloxy-5-methoxy-1H-indole-2-carboxylic acid;
1-(2-Carboxymethoxy-4-methoxy-benzyl)-5,6-dimethoxy-3-(3-methoxy-phenylsulfanyl)-1H-indole-2-carboxylic acid;
1-Benzo[1,3]dioxol-5-ylmethyl-3-(benzo[1,3]dioxol-5-ylsulfanyl)-5,6-dimethoxy-1H-indole-2-carboxylic acid;
1-Benzo[1,3]dioxol-5-ylmethyl-3-(3,4,5-trimethoxy-phenylsulfanyl)-6-benzyloxy-5-methoxy-1H-indole-2-carboxylic acid;
5-Benzo[1,3]dioxol-5-ylmethyl-7-(3,4,5-trimethoxy-phenylsulfanyl)-5H-[1,3]dioxolo[4,5-f]indole-6-carboxylic acid;
5-Benzo[1,3]dioxol-5-ylmethyl-7-(benzo[1,3]dioxol-5-ylsulfanyl)-5H-[1,3]dioxolo[4,5-f]indole-6-carboxylic acid;
[2S -(2Alpha,3beta,4alpha)]4-(1,3-benzodioxol-5-yl)-1-[2-(dibutylamino)-2-oxoethyl]-2-(4-methoxyphenyl)-3-pyrrolidinecarboxylic acid;
3-Pyrrolidinecarboxylic acid, 4-(1,3-benzodioxol-5-yl)-1-[2-(dibutylamino)-2-oxoethyl]-2-(4-methoxyphenyl)-, sodium salt, (2R,3R,4S)-rel-;
N-[6-(2-Hydroxyethoxy)-5-(2-methoxyphenoxy)-2-[2-(1H-tetrazol-5-yl)-4-pyridinyl]-4-pyrimidinyl]-5-(1-methylethyl)-2-pyridinesulfonamide;
5-(Dimethylamino)-N-(3,4-dimethyl-5-isoxazolyl)-1-naphthalenesulfonamide;
4-(1,1-Dimethylethyl)-N-[6-(2-hydroxyethoxy)-5-(2-methoxyphenoxy)[2,2′-bipyrimidin]-4-yl]benzenesulfonamide;
Benzenesulfonamide, 4-(1,1-dimethyl)-N-[6-(2-hydroxyethoxy)-5-(3-methoxyphenoxy)4-pyrimidinyl]-;
[N-cis-2,6-Dimethylpiperidinocarbonyl-L-.gamma.-methylleucyl-D-1-methoxycarbonyltryptophanyl-D-norleucine];
[5S-[5Alpha,6beta,7alpha(R*)]]-5-(1,3-benzodioxol-5-yl)-2-butyl-7-[2-(2-carboxypropyl)-4-methoxyphenyl]-6,7-dihydro-5H-cyclopenta[b]pyridine-6-carboxylic acid;
5S-(5alpha,6beta,7alpha(R*)))-2-butyl-5-(1,3-benzodiox-ol-5-yl)-7-((2-carboxypropyl)-4-methoxyphenyl)-6-dihydro-5H-cyclopenta(b)pyridine-6-carboxylic acid (J-104120);
[5S-[5Alpha,6beta,7alpha(R*)]]-5-(1,3-benzodioxol-5-yl)-2-butyl-7-[2-(2-carboxypropyl)-4-methoxyphenyl]-6,7-dihydro-5H-cyclopenta[b]pyridine-6-carboxylic acid (L-753037);
(S)-Alpha-[(4,6-Dimethoxy-2-pyrimidinyl)oxy]-beta-methoxy-beta-phenyl benzenepropanoic acid;
Alpha-((4,6-dimethoxy-2-pyrimidinyl)oxy)-beta-methoxy-beta-phenyl-benzenepropanoic acid (LU-1 27043);
2-(4,6-Dimethoxypyrimidin-2-yloxy)-3-ethoxy-3,3-diphenylpropionic acid;
N-[6-(2-Hydroxyethoxy)-5-(2-methoxyphenoxy)-2-[2-(1H-tetrazol-5-yl)-4-pyridinyl]-4-pyrimidinyl]-5-methyl-2-pyridinesulfonamide;
2-Pyridinesulfonamide, N-[6-(2-hydroxyethoxy)-5-(2-methoxyphenoxy)-2-[2-(1H-tetrazol-5-yl)-4-pyridinyl]-4-pyrimidinyl]-5-methyl-;
27-O-3-[2-(3-Carboxy-acryloylamino)-5-hydroxyphenyl]-acryloyloxy myricerone;
N-[6-[2-[(5-Bromo-2-pyrimidinyl)oxy]ethoxy]-5-(4-methylphenyl)-4-pyrimidinyl]-4-(2-hydroxy-1,1-dimethylethyl)-benzenesulfonamide monosodium;
4-tert-Butyl-N-(5-(4-methylphenyl)-6-(2-(5-(3-thienyl)pyrimidin-2-yloxy)ethoxy)pyrimidin-4-yl)-benzenesulfonamide (T-0115);
Cyclo[4-oxo-4-(4-phenyl-1-piperazinyl)-L-2-aminobutanoyl-L-alpha-aspartyl-D-2-(2-thienyl)glycyl-L-leucyl-D-tryptophyl-D-alpha-aspartyl]disodium salt;
N-(4-Chloro-3-methyl-5-isoxazolyl)-2-[(6-methyl-1,3-benzodioxol-5-yl)acetyl]-3-thiophenesulfonamide;
3-Thiophenesulfonamide, N-(4-chloro-3-methyl-5-isoxazolyl)-2-[(6-methyl-1,3-benzodioxol-5-yl)acetyl]- (IPI-1040);
3-Thiophenesulfonamide, N-(4-chloro-3-methyl-5-isoxazolyl)-2-[(6-methyl-1,3-benzodioxol-5-yl)acetyl]- (IPI-1251); and
3-Thiophenesulfonamide, N-(4-chloro-3-methyl-5-isoxazolyl)-2-[(6-methyl-1,3-benzodioxol-5-yl)acetyl]- (TBC-11241).

12. The combination of claim 1 wherein the endothelin receptor antagonist is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, or a pharmaceutically acceptable salt thereof.

13. The combination of claim 1 wherein the endothelin receptor antagonist is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt.

14. The combination of claim 1 wherein the the endothelin receptor antagonist is 4-(3,5-dimethyl-phenyl)-1,1-dioxo-2-(2-trifluoromethyl-phenyl)-1,2-dihydro-1λ⁶-benzo[e][1,2]thiazine-3-carboxylic acid, or a pharmaceutically acceptable salt thereof

15. The combination of claim 1 wherein the the endothelin receptor antagonist is 4-(3,5-dimethyl-phenyl)-1,1-dioxo-2-(2-trifluoromethyl-phenyl)-1,2-dihydro-1λ⁶-benzo[e][1,2]thiazine-3-carboxylic acid.

16. The combination of claim 1 wherein the analgesic is an NSAID.

17. The combination of claim 1 wherein the analgesic is an NSAID selected from:
Naproxen;
Naproxen sodium;
Ibuprofen;
Acetaminophen;
Aspirin;
Sulindac;
Tolmetin;
Piroxicam;
Mefenamic acid;
Phenylbutazone;
Fenoprofen;
Ketoprofen;
Suprofen;
Diflunisal;
Celecoxib;
Meloxicam; and
(Z)-5-[[3,5-Bis(1,1-dimethylethyl)-4-hydroxyphenyl]methylene]-2-irnino-4-thiazolidinone methanesulfonate (1:1).

18. The combination of claim 1 wherein the analgesic is an NMDA receptor antagonist.

19. The combination of claim 1 wherein the analgesic is an NMDA receptor antagonist selected from:
1H-Indole-2-carboxylic acid, 4,6-dichloro-3-[3-oxo-3-(phenylamino)-1-propenyl]-, (E)-(GV-150526);
1-Piperidineethanol, .alpha.-(4-hydroxyphenyl)-.beta.-methyl-4-(phenylmethyl)- (Ifenprodil);
ACEA 1168; and
(1S,2S)-1-(4-Hydroxyphenyl)-2-(4-hydroxy-4-phenylpiperidine)-1-propanol.

20. The combination of claim 1 wherein the analgesic is an NK₁ receptor antagonist.

21. The combination of claim 1 wherein the analgesic is an NK₁ receptor antagonist named:
[2-(1H-indol-3-yl)-1-methyl-1-(1-phenyl-ethylcarbamoyl)-ethyl]-carbamic acid benzofuran-2-ylmethyl ester.

22. The combination of claim 1 wherein the analgesic is an opioid.

23. The combination of claim 1 wherein the analgesic is an opioid selected from:
Codeine;
Morphine;
Hydromorphone;
Levorphanol;
Methadone;
Oxycodone;
Hydrocodone;
Pentazocine;
Nalbuphine;
Butorphanol;
Hydromorphone; and
Naloxone.

24. The combination of claim 1 wherein the endothelin receptor antagonist is 2R-(4-methoxyphenyl)-4S-(1,3-benzodioxol-5-yl)-1-(N,N-di(n-butyl)-aminocarbonyl-methyl)-pyrrolidine-3R-carboxylic acid (ABT-627).

25. A combination of claim 1 comprising a pain alleviating effective amount of an endothelin antagonist or a pharmaceutically acceptable salt thereof and one or two antiepileptics compounds having pain alleviating properties, and pharmaceutically acceptable salts thereof.

26. The combination of claim 1 wherein the endothelin receptor antagonist is selected from:
4-(3,5-Dimethyl-phenyl)-1,1-dioxo-2-(2-trifluoromethyl-phenyl)-1,2-dihydro-1λ⁶-benzo[e][1,2]thiazine-3-carboxylic acid,
4-(7-Ethyl-1,3-benzodiozol-5-yl)-2-[2-(trifluoromethyl)phenyl]-2H-1,2-dihydro-1,2-benzothiazine-3-carboxylic acid; 1,1-dioxide, and
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, potassium salt; and
the antiepileptic compound having pain alleviating properties is selected from gabapentin, pregabalin, and 3-(1-aminomethyl-cyclohexylmethyl)-4H-[1,2,4]oxadiazol-5-one hydrochloride.

27. The combination of claim 1 wherein the endothelin receptor antagonist is 4-(7-ethyl-1,3-benzodioxol-5-yl)-2-[2-trifluoromethyl)phenyl]-2H-1,2-dihydro-1,2-benzothiazine-3-carboxylic acid 1,1-dioxide potassium salt and the antiepileptic compound having pain alleviating properties is gabapentin.

28. The combination of claim 1 wherein the endothelin receptor antagonist is 4-(7-ethyl-1,3-benzodioxol-5-yl)-2-[2-trifluoromethyl)phenyl]-2H-1,2-dihydro-1,2-benzothiazine-3-carboxylic acid 1,1-dioxide potassium salt and the antiepileptic compound having pain alleviating properties is pregabalin.

29. The combination of claim 1 wherein the endothelin receptor antagonist is 4-(7-ethyl-1,3-benzodioxol-5-yl)-2-[2-trifluoromethyl)phenyl]-2H-1,2-dihydro-1,2-benzothiazine-3-carboxylic acid 1,1-dioxide potassium salt and the antiepileptic compound having pain alleviating properties is 3-(1-aminomethyl-cyclohexylmethyl)-4H-[1,2,4]oxadiazol-5-one hydrochloride.

30. A combination of claim 1 comprising a pain alleviating effective amount of an endothelin antagonist or a pharmaceutically acceptable salt thereof and one or two analgesics, and pharmaceutically acceptable salts thereof.

31. A combination of claim 1 comprising a pain alleviating effective amount of an endothelin antagonist or a pharmaceutically acceptable salt thereof and one or two analgesics, and pharmaceutically acceptable salts thereof wherein the analgesic is an opioid analgesic.

32. The combination of claim 1 wherein the endothelin receptor antagonist is selected from:
4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, potassium salt; and
the analgesic is an opioid analgesic selected from:
Codeine;
Morphine;
Hydromorphine;
Levorphanol;
Methadone;
Oxycodone;
Hydrocodone;
Pentazocine;
Nalbuphine;
Butorphanol;
Hydromorphone; and
Naloxone.

33. A combination of claim 1 comprising a pain alleviating effective amount of an endothelin antagonist or a pharmaceutically acceptable salt thereof and one or two analgesics, and pharmaceutically acceptable salts thereof wherein the analgesic is a nonopioid analgesic.

34. The combination of claim 1 wherein the analgesic is an NSAID nonopioid analgesic.

35. The combination of claim 1 wherein the endothelin receptor antagonist is selected from:
4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, potassium salt; and
the analgesic is an NSAID nonopioid analgesic selected from:
Naproxen;
Naproxen sodium;
Ibuprofen;
Acetaminophen;
Aspirin;
Sulindac;
Tolmetin;
Piroxicam;
Mefenarnic acid;
Phenylbutazone;
Fenoprofen;
Ketoprofen;
Suprofen;
Diflunisal;
Celecoxib;
Meloxicam; and
(Z)-5-[[3,5-Bis(1,1-dimethylethyl)-4-hydroxyphenyl]-methylene]-2-imino-4-thiazolidinone methanesulfonate (1:1).

36. The combination of claim 1 wherein the analgesic is an NMDA receptor antagonist nonopioid analgesic.

37. The combination of claim 1 wherein the endothelin receptor antagonist is selected from:
4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, potassium salt; and
the analgesic is a nonopioid analgesic selected from:
1H-Indole-2-carboxylic acid, 4,6-dichloro-3-[3-oxo-3-(phenylamino)-1-propenyl]-, (E)-(GV-150526);
1-Piperidineethanol, .alpha.-(4-hydroxyphenyl)-.beta.-methyl-4-(phenylmethyl)- (Ifenprodil);
ACEA1168;and
(1S,2S)-1-(4-Hydroxyphenyl)-2-(4-hydroxy-4-phenylpiperidine)-1-propanol.

38. The combination of claim 1 wherein the endothelin receptor antagonist is selected from:
4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and
4-(7-Ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt;
and the analgesic is a nonopioid analgesic named:
[2-(1H-Indol-3-yl)-1-methyl-1-(1-phenyl-ethylcarbamoyl)-ethyl]carbamic acid benzofuran-2-ylmethyl ester.

39. A combination of claim 1 comprising a pain alleviating effective amount of an endothelin antagonist or a pharmaceutically acceptable salt thereof and one antiepileptic compound having pain alleviating properties and one analgesic.

40. A combination of claim 1 comprising a pain alleviating effective amount of an endothelin antagonist or a pharmaceutically acceptable salt thereof and one antiepileptic compound having pain alleviating properties and one analgesic wherein the analgesic is an opioid analgesic.

41. The combination of claim 1 wherein the endothelin receptor antagonist is selected from:
4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, potassium salt;
the antiepileptic is selected from gabapentin, pregabalin, and 3-(1-aminomethyl-cyclohexylmethyl)-4H-[1,2,4]oxadiazol-5-one hydrochloride; and
the analgesic is an opioid analgesic selected from:
Codeine;
Morphine;
Hydromorphine;
Levorphanol;
Methadone;
Oxycodone;
Hydrocodone;
Pentazocine;
Nalbuphine;
Butorphanol;
Hydromorphone; and
Naloxone.

42. A combination of claim 1 comprising a pain alleviating effective amount of an endothelin receptor antagonist or a pharmaceutically acceptable salt thereof and one antiepileptic compound having pain alleviating properties and one analgesic wherein the analgesic is a nonopioid analgesic.

43. The combination of claim 1 wherein the endothelin receptor antagonist is selected from:
4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, potassium salt;
the antiepileptic compound having pain alleviating properties is selected from gabapentin, pregabalin, and 3-(1-aminomethyl-cyclohexylmethyl)-4H-[1,2,4]oxadiazol-5-one hydrochloride; and
the analgesic is a nonopioid analgesic selected from:
Naproxen;
Naproxen sodium;
Ibuprofen;
Acetaminophen;
Aspirin;
Sulindac;
Tolmetin;
Piroxicam;
Mefenamic acid;
Phenylbutazone;
Fenoprofen;
Ketoprofen;
Suprofen;
Diflunisal;
Celecoxib;
Meloxicam;
(Z)-5-[[3,5-Bis(1,1-dimethylethyl)-4-hydroxyphenyl]-methylene]-2-imino4-thiazolidinone methanesulfonate (1:1);
[2-(1H-Indol-3-yl)-1-methyl-1-(1-phenyl-ethylcarbamoyl)-ethyl]-carbamic acid benzofuran-2-ylmethyl ester;
2H-Indole-2-carboxylic acid, 4,6-dichloro-3-[3-oxo-3-(phenylamino)-1-propenyl]-, (E)-(GV-150526);
1-Piperidineethanol, .alpha.-(⁴-hydroxyphenyl)-.beta.-methyl-4,-(phenylmethyl)- (Ifenprodil);
ACEA 1168; and
(1S,2S)-1-(4-Hydroxyphenyl)-2-(4-hydroxy-4-phenylpiperidine)-1-propanol.

44. A combination of claim 1 comprising a pain alleviating effective amount of an endothelin antagonist or a pharmaceutically acceptable salt thereof and two analgesics wherein one analgesic is an opioid analgesic and one analgesic is a nonopioid analgesic.

45. The combination of claim 1 wherein the endothelin receptor antagonist is selected from:
4-(3,5-Dimethyl-phenyl)-1,1-dioxo-2-(2-trifluoromethyl-phenyl)-1,2-dihydro-1λ⁶-benzo[e][1,2]thiazine-3-carboxylic acid;
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide; and
4-(7-Ethyl-1,3-benzodiozol-5-yl)-2-[2-(trifluoromethyl)phenyl]-2H-1,2-dihydro-1,2-benzothiazine-3-carboxylic acid, 1,1-dioxide, potassium salt;
the one analgesic is an opioid analgesic selected from:
Codeine;
Morphine;
Hydromorphine;
Levorphanol;
Methadone;
Oxycodone;
Hydrocodone;
Pentazocine;
Nalbuphine;
Butorphanol;
Hydromorphone; and
Naloxone; and
one analgesic is a nonopioid analgesic selected from:
Naproxen;
Naproxen sodium;
Ibuprofen;
Acetaminophen;
Aspirin;
Sulindac;
Tolmetin;
Piroxicam;
Mefenamic acid;
Phenylbutazone;
Fenoprofen;
Ketoprofen;
Suprofen;
Diflunisal;
Celecoxib;
Meloxicam;
(Z)-5-[[3,5-Bis(1,1-dimethylethyl)-4-hydroxyphenyl]-methylene]-2-imino-4-thiazolidinone methanesulfonate (1:1);
[2-(1H-Indol-3-yl)-1-methyl-1-(1-phenyl-ethylcarbamoyl)-ethyl]-carbamic acid benzofuran-2-ylmethyl ester;
1H-Indole-2-carboxylic acid, 4,6-dichloro-3-[3-oxo-3-(phenylamino)-1-propenyl]-, (E)-(GV-150526);
1-Piperidineethanol, .alpha.-(4-hydroxyphenyl)-.beta.-methyl-4-(phenylmethyl)- (Ifenprodil);
ACEA 1168; and
(1S,2S)-1-(4-Hydroxyphenyl)-2-(4-hydroxy-4-phenylpiperidine)-1-propanol.

46. A method of treating pain in a mammal suffering therefrom, comprising administering a pain alleviating effective amount of a combination of claim 1.

47. A method according to claim 46 wherein the pain being treated is selected from the group consisting of: centrally mediated pain, peripherally mediated pain, structural pain, soft tissue pain, injury-related pain, progressive disease-related pain, and neuropathic pain.

48. The method according to claim 46 wherein the pain being treated is diabetic peripheral neuropathy.

49. The method according to claim 46 wherein the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is gabapentin.

50. The method according to claim 46 wherein the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is pregabalin.

51. The method according to claim 46 wherein the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is 3-(1-aminomethyl-cyclohexylmethyl)-4H-[1,2,4]oxadiazol-5-one hydrochloride.

52. The method according to claim 46 wherein the pain being treated is diabetic peripheral neuropathy and the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is gabapentin.

53. The method according to claim 46 wherein the pain being treated is diabetic peripheral neuropathy and the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is pregabalin.

54. The method according to claim 46 wherein the pain being treated is diabetic peripheral neuropathy and the combination administered comprises an endothelin receptor antagonist which is 4-(7-ethyl-1,3-benzodioxol-5-yl)-2-[2-(trifluoromethyl)phenyl]-2H-1,2-dihydro-1,2-benzothiazine-3-carboxylic acid, 1,1-dioxide, potassium salt, and an antiepileptic compound having pain alleviating properties which is 3-(1-5 aminomethyl-cyclohexylmethyl)-4H-[1,2,4]oxadiazol-5-one hydrochloride.

55. The method according to claim 46 wherein the combination administered comprises an endothelin receptor antagonist which is 4-(3,5-dimethyl-phenyl)-1,1-dioxide-2-(2-trifluoromethyl-phenyl)-1,2-dihydro-1λ⁶-benzo[e][1,2]thiazine-3-carboxylic acid, and an antiepileptic compound having pain alleviating properties which is gabapentin.

56. The method according to claim 46 wherein the combination administered comprises an endothelin receptor antagonist which is 4-(3,5-dimethyl-phenyl)-1,1-dioxide-2-(2-trifluoromethyl-phenyl)-1,2-dihydro-1λ⁶-benzo[e][1,2]thiazine-3-carboxylic acid, and an antiepileptic compound having pain alleviating properties which is pregabalin.

57. The method according to claim 46 wherein the combination administered comprises an endothelin receptor antagonist which is 4-(3,5-dimethyl-phenyl)-1,1-dioxide-2-(2-trifluoromethyl-phenyl)-1,2-dihydro-1λ⁶-benzo[e][1,2]thiazine-3-carboxylic acid, and an antiepileptic compound having pain alleviating properties which is 3-(1-aminomethyl-cyclohexylmethyl)-4H-[1,2,4]oxadiazol-5-one hydrochloride.

58. A pharmaceutical composition comprising a pain alleviating effective amount of a combination of claim 1, and a pharmaceutically acceptable excipient, diluent, or carrier.

59. A method of treating pain in a mammal suffering therefrom, comprising administering a pain alleviating effective amount of a pharmaceutical composition of claim 58.
